SEQUENCE LISTING (141 sequences)

SEQ ID NO: 1 - protein, 605 aa, Rhizopus microsporus
MKLMNPSMKAYVFFILSYFSLLVSSAAVPTSAAVQVESYNYDGTTFSGRIFVKNIAYSKVVTVIYSDGSDNWNNNNNKVSAAYSEAISGSNYEYWTFSAKLSGIKQFYVKYEVSGSTYYDNNGTKNYQVQATSATSTTATATTTTATGTTTTSTGPTSTASVSFPTGNSTISSWIKNQEEISRFAMLRNINPPGSATGFIAASLSTAGPDYYYSWTRDSALTANVIAYEYNTTFTGNTTLLKYLKDYVTFSVKSQSVSTVCNCLGEPKFNADGSSFTGPWGRPQNDGPAERAVTFMLIADSYLTQTKDASYVTGTLKPAIFKDLDYVVSVWSNGCYDLWEEVNGVHFYTLMVMRKGLILGADFAARNGDSSRASTYKQTASTMESKISSFWSDSNNYVQVSQSVTAGVSKKGLDVSTLLAANIGSLPDGFFTPGSEKILATAVALENAFASLYPINSNLPSYLGNSIGRYPEDTYNGNGNSQGNPWFLAVNAYAELYYRAIKEWISNGKVTVSNISLPFFKKFDSSATSGKTYTAGTSDFNNLAQNIALGADRFLSTVKFHAYTNGSLSEEYDRSTGMSTGARDLTWSHASLITVAYAKAGSPAA

SEQ ID NO: 2 - DNA, 4037 bp, synthetic construct
  source 1..4037: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatggccaagttattgattgctggagattgggaggccggtgctacatcagatgtgctaagagacaaatttcacgacgcaccattcggagaaatggacgttgcctcagccgatcaagtagacagagccgtggctggtgcagtggaaggtcaacaacaagattctttgtcgccatatgatagatataggattttgatggcggcatctagaattgtcagagatagacaggaaacctttgtagaattgatgagagacgaagcaggttttaccagagccgatggtgagaatgaggtacgtagatgtgctcaaacgcttgaattgagcgcagaagaagcaaaaatgcttacaggagatttggtcccaatgttgagtgctgatggtgctaaggatagaatgggatttactcaaagagctccaagaggggtcatttgtgctataactccgtttaactccccactgaatacgttagcgcataaggtcggtcctgctcttgcgggagggaactctgtggttgtcaagccttccaatgtcactcctttaagtgccgtcgaactttgcagaatcttactggaagcaggcttacctcctaagcttttagcattagtacacggtaggggtagtgttattgggagacaactgttggatgatcaaaggatcgcctactacgcattcacgggatccacctctgtaggtagagaaattcagtctgctgctggtttaagaggtgttcaattggaattgggatccatagcttctaccattgtttgtcatgacgcagacttagacaaggcagttcctaagatcgtaaatgcaggattcagaaaggcagggcaagtctgcaccagcgtacagcgtttacacgtggacgcaagaattctagatgacctaagaggtagattgcttacagctataggtgcattccctgctggtgatccatcagaccctgaaactagggtggggccaatgatttccagagcagatgcagaacgtgctgaagcttggatcgcagaggctatggctgctggcgcgagaatcgccgtcggtggaaagcgtgaagggtctgtcgtggaaccaaccgtgttggaaggtgtaacgcccgaaatgaaggtgtatgccgatgaaatttttgcgccagtcatgtgcctgattccttttgatactttggacgaagctgtaactagagccaacgcaagcgattttggactagctgcaggattgttcacaagtgacttaggcacagcactaagaaccgctccgagattgagatttggtggtgtccatgtgaacgaagctagttcggctagaatagatgcgatgccattcggtggagtaaaagattcgggatacggtagagaaggtccagcttacgccgttagagaaatgactgaggaaagaccaattacaatcagctattaacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 3 - DNA, 3826 bp, synthetic construct
  source 1..3826: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttaatagctgattgtaattggtctttcctcagtcatttctctaacggcgtaagctggaccttctctaccgtatcccgaatcttttactccaccgaatggcatcgcatctattctagccgaactagcttcgttcacatggacaccaccaaatctcaatctcggagcggttcttagtgctgtgcctaagtcacttgtgaacaatcctgcagctagtccaaaatcgcttgcgttggctctagttacagcttcgtccaaagtatcaaaaggaatcaggcacatgactggcgcaaaaatttcatcggcatacaccttcatttcgggcgttacaccttccaacacggttggttccacgacagacccttcacgctttccaccgacggcgattctcgcgccagcagccatagcctctgcgatccaagcttcagcacgttctgcatctgctctggaaatcattggccccaccctagtttcagggtctgatggatcaccagcagggaatgcacctatagctgtaagcaatctacctcttaggtcatctagaattcttgcgtccacgtgtaaacgctgtacgctggtgcagacttgccctgcctttctgaatcctgcatttacgatcttaggaactgccttgtctaagtctgcgtcatgacaaacaatggtagaagctatggatcccaattccaattgaacacctcttaaaccagcagcagactgaatttctctacctacagaggtggatcccgtgaatgcgtagtaggcgatcctttgatcatccaacagttgtctcccaataacactacccctaccgtgtactaatgctaaaagcttaggaggtaagcctgcttccagtaagattctgcaaagttcgacggcacttaaaggagtgacattggaaggcttgacaaccacagagttccctcccgcaagagcaggaccgaccttatgcgctaacgtattcagtggggagttaaacggagttatagcacaaatgacccctcttggagctctttgagtaaatcccattctatccttagcaccatcagcactcaacattgggaccaaatctcctgtaagcatttttgcttcttctgcgctcaattcaagcgtttgagcacatctacgtacctcattctcaccatcggctctggtaaaacctgcttcgtctctcatcaattctacaaaggtttcctgtctatctctgacaattctagatgccgccatcaaaatcctatatctatcatatggcgacaaagaatcttgttgttgaccttccactgcaccagccacggctctgtctacttgatcggctgaggcaacgtccatttctccgaatggtgcgtcgtgaaatttgtctcttagcacatctgatgtagcaccggcctcccaatctccagcaatcaataacttggccatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 4 - protein, 470 aa, Roseisalinus antarcticus
MAKLLIAGDWEAGATSDVLRDKFHDAPFGEMDVASADQVDRAVAGAVEGQQQDSLSPYDRYRILMAASRIVRDRQETFVELMRDEAGFTRADGENEVRRCAQTLELSAEEAKMLTGDLVPMLSADGAKDRMGFTQRAPRGVICAITPFNSPLNTLAHKVGPALAGGNSVVVKPSNVTPLSAVELCRILLEAGLPPKLLALVHGRGSVIGRQLLDDQRIAYYAFTGSTSVGREIQSAAGLRGVQLELGSIASTIVCHDADLDKAVPKIVNAGFRKAGQVCTSVQRLHVDARILDDLRGRLLTAIGAFPAGDPSDPETRVGPMISRADAERAEAWIAEAMAAGARIAVGGKREGSVVEPTVLEGVTPEMKVYADEIFAPVMCLIPFDTLDEAVTRANASDFGLAAGLFTSDLGTALRTAPRLRFGGVHVNEASSARIDAMPFGGVKDSGYGREGPAYAVREMTEERPITISY

SEQ ID NO: 5 - DNA, 1413 bp, synthetic construct
  source 1..1413: mol_type = other DNA
atggccaagttattgattgctggagattgggaggccggtgctacatcagatgtgctaagagacaaatttcacgacgcaccattcggagaaatggacgttgcctcagccgatcaagtagacagagccgtggctggtgcagtggaaggtcaacaacaagattctttgtcgccatatgatagatataggattttgatggcggcatctagaattgtcagagatagacaggaaacctttgtagaattgatgagagacgaagcaggttttaccagagccgatggtgagaatgaggtacgtagatgtgctcaaacgcttgaattgagcgcagaagaagcaaaaatgcttacaggagatttggtcccaatgttgagtgctgatggtgctaaggatagaatgggatttactcaaagagctccaagaggggtcatttgtgctataactccgtttaactccccactgaatacgttagcgcataaggtcggtcctgctcttgcgggagggaactctgtggttgtcaagccttccaatgtcactcctttaagtgccgtcgaactttgcagaatcttactggaagcaggcttacctcctaagcttttagcattagtacacggtaggggtagtgttattgggagacaactgttggatgatcaaaggatcgcctactacgcattcacgggatccacctctgtaggtagagaaattcagtctgctgctggtttaagaggtgttcaattggaattgggatccatagcttctaccattgtttgtcatgacgcagacttagacaaggcagttcctaagatcgtaaatgcaggattcagaaaggcagggcaagtctgcaccagcgtacagcgtttacacgtggacgcaagaattctagatgacctaagaggtagattgcttacagctataggtgcattccctgctggtgatccatcagaccctgaaactagggtggggccaatgatttccagagcagatgcagaacgtgctgaagcttggatcgcagaggctatggctgctggcgcgagaatcgccgtcggtggaaagcgtgaagggtctgtcgtggaaccaaccgtgttggaaggtgtaacgcccgaaatgaaggtgtatgccgatgaaatttttgcgccagtcatgtgcctgattccttttgatactttggacgaagctgtaactagagccaacgcaagcgattttggactagctgcaggattgttcacaagtgacttaggcacagcactaagaaccgctccgagattgagatttggtggtgtccatgtgaacgaagctagttcggctagaatagatgcgatgccattcggtggagtaaaagattcgggatacggtagagaaggtccagcttacgccgttagagaaatgactgaggaaagaccaattacaatcagctattaa

SEQ ID NO: 6 - DNA, 4019 bp, synthetic construct
  source 1..4019: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatggcagaaagtttgcgtttgatcaccccagttgacggatctgtctatgccgaacgtccactagcgaccttagcggaggcagaacaagcagtcgctagagcaagagttgcacaagtggcttggagaaaatgccctgttggtgagagagctgcgctattgggtagattcgtagatgcttttttggcaagaggtgaagcaattgccgaggagttaacctggcagattggtaggccaatcagtcaaagtcctggtgagctgagaggcttcgaagaaagggccagacacatgattgctattgcggctgaatcattggccgatgttagagttggcgaaatcccgggttttactaggtttattagaagagagcccctaggagtcgttttatctattcctgcttggaactttccttatttaactgcggtcaactccatcgtgccagcattgttagccggaaatgcagttattctgaagcacagtgctcaaacggctctttgtggggaaagattcgctgctgcatttgcagaagctggtctaccagcgggcttatttagaaatataaatctagatcacgctaccacagaagccttgatagcggctgacggggtagactacgtatcctttacgggcagtgttggaggtggtcatagaatccaagccgccgcttcccaaagatttattgctactggtttggagttaggtggtaaagatccagcttatgttagagcggatgccgatctagattttgctgtagaaaaccttgttgatggtgcatttttcaattctggccaaagctgttgcggcattgaaagaatttatgttcacgaagctttatatgatagattcacagaggcatttgctgaaactgtaagaggttatagactaggtaatccactggaaccgtcggtcaacttaggacctatggttagaacgagtgctgctgatgctgttagagaacaagtccgtgccgctactgctgctggagccagagctttaatagaccctggtcactttccggcgaatgcagacggtacaccctaccttgctccacaagtgcttattgacgtcgatcaccaaatgagtgtcatgagagacgagagctttgggccagtcgtgggtataatgaaggttccagacgacgccgcagcattagaacttattaatgactccccttacggcttaacagcgtctatctggaccggtgacttggatgcaggtctagcattgggcgatgaagtacaaacgggtacgttgttcatgaacagatgtgattacttagatcctgcattagcttgggtgggtgtaaaggacagtggacgtggttgtaccttatccagggtaggttatgaacatcttaccagaccaaagtctttccacatgagggaaggtaataagtaacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 7 - DNA, 3808 bp, synthetic construct
  source 1..3808: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttacttattaccttccctcatgtggaaagactttggtctggtaagatgttcataacctaccctggataaggtacaaccacgtccactgtcctttacacccacccaagctaatgcaggatctaagtaatcacatctgttcatgaacaacgtacccgtttgtacttcatcgcccaatgctagacctgcatccaagtcaccggtccagatagacgctgttaagccgtaaggggagtcattaataagttctaatgctgcggcgtcgtctggaaccttcattatacccacgactggcccaaagctctcgtctctcatgacactcatttggtgatcgacgtcaataagcacttgtggagcaaggtagggtgtaccgtctgcattcgccggaaagtgaccagggtctattaaagctctggctccagcagcagtagcggcacggacttgttctctaacagcatcagcagcactcgttctaaccataggtcctaagttgaccgacggttccagtggattacctagtctataacctcttacagtttcagcaaatgcctctgtgaatctatcatataaagcttcgtgaacataaattctttcaatgccgcaacagctttggccagaattgaaaaatgcaccatcaacaaggttttctacagcaaaatctagatcggcatccgctctaacataagctggatctttaccacctaactccaaaccagtagcaataaatctttgggaagcggcggcttggattctatgaccacctccaacactgcccgtaaaggatacgtagtctaccccgtcagccgctatcaaggcttctgtggtagcgtgatctagatttatatttctaaataagcccgctggtagaccagcttctgcaaatgcagcagcgaatctttccccacaaagagccgtttgagcactgtgcttcagaataactgcatttccggctaacaatgctggcacgatggagttgaccgcagttaaataaggaaagttccaagcaggaatagataaaacgactcctaggggctctcttctaataaacctagtaaaacccgggatttcgccaactctaacatcggccaatgattcagccgcaatagcaatcatgtgtctggccctttcttcgaagcctctcagctcaccaggactttgactgattggcctaccaatctgccaggttaactcctcggcaattgcttcacctcttgccaaaaaagcatctacgaatctacccaatagcgcagctctctcaccaacagggcattttctccaagccacttgtgcaactcttgctctagcgactgcttgttctgcctccgctaaggtcgctagtggacgttcggcatagacagatccgtcaactggggtgatcaaacgcaaactttctgccatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 8 - protein, 464 aa, Oceanibacterium hippocampi
MAESLRLITPVDGSVYAERPLATLAEAEQAVARARVAQVAWRKCPVGERAALLGRFVDAFLARGEAIAEELTWQIGRPISQSPGELRGFEERARHMIAIAAESLADVRVGEIPGFTRFIRREPLGVVLSIPAWNFPYLTAVNSIVPALLAGNAVILKHSAQTALCGERFAAAFAEAGLPAGLFRNINLDHATTEALIAADGVDYVSFTGSVGGGHRIQAAASQRFIATGLELGGKDPAYVRADADLDFAVENLVDGAFFNSGQSCCGIERIYVHEALYDRFTEAFAETVRGYRLGNPLEPSVNLGPMVRTSAADAVREQVRAATAAGARALIDPGHFPANADGTPYLAPQVLIDVDHQMSVMRDESFGPVVGIMKVPDDAAALELINDSPYGLTASIWTGDLDAGLALGDEVQTGTLFMNRCDYLDPALAWVGVKDSGRGCTLSRVGYEHLTRPKSFHMREGNK

SEQ ID NO: 9 - DNA, 1395 bp, synthetic construct
  source 1..1395: mol_type = other DNA
atggcagaaagtttgcgtttgatcaccccagttgacggatctgtctatgccgaacgtccactagcgaccttagcggaggcagaacaagcagtcgctagagcaagagttgcacaagtggcttggagaaaatgccctgttggtgagagagctgcgctattgggtagattcgtagatgcttttttggcaagaggtgaagcaattgccgaggagttaacctggcagattggtaggccaatcagtcaaagtcctggtgagctgagaggcttcgaagaaagggccagacacatgattgctattgcggctgaatcattggccgatgttagagttggcgaaatcccgggttttactaggtttattagaagagagcccctaggagtcgttttatctattcctgcttggaactttccttatttaactgcggtcaactccatcgtgccagcattgttagccggaaatgcagttattctgaagcacagtgctcaaacggctctttgtggggaaagattcgctgctgcatttgcagaagctggtctaccagcgggcttatttagaaatataaatctagatcacgctaccacagaagccttgatagcggctgacggggtagactacgtatcctttacgggcagtgttggaggtggtcatagaatccaagccgccgcttcccaaagatttattgctactggtttggagttaggtggtaaagatccagcttatgttagagcggatgccgatctagattttgctgtagaaaaccttgttgatggtgcatttttcaattctggccaaagctgttgcggcattgaaagaatttatgttcacgaagctttatatgatagattcacagaggcatttgctgaaactgtaagaggttatagactaggtaatccactggaaccgtcggtcaacttaggacctatggttagaacgagtgctgctgatgctgttagagaacaagtccgtgccgctactgctgctggagccagagctttaatagaccctggtcactttccggcgaatgcagacggtacaccctaccttgctccacaagtgcttattgacgtcgatcaccaaatgagtgtcatgagagacgagagctttgggccagtcgtgggtataatgaaggttccagacgacgccgcagcattagaacttattaatgactccccttacggcttaacagcgtctatctggaccggtgacttggatgcaggtctagcattgggcgatgaagtacaaacgggtacgttgttcatgaacagatgtgattacttagatcctgcattagcttgggtgggtgtaaaggacagtggacgtggttgtaccttatccagggtaggttatgaacatcttaccagaccaaagtctttccacatgagggaaggtaataagtaa

SEQ ID NO: 10 - DNA, 4061 bp, synthetic construct
  source 1..4061: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatggttgtcaatgtatataatttctatgttaatggcgaatggagggaatcttctagtggtgaaaccattgaaatctccagcccatatttgcatgaggtgattggacaagtgcaagccattacatgtggggaggtagatgaagctatagcttcggcaaaagaagcgcaaaagtcatgggcaaaagcatccttacaagaccgtgccaagtacttatacaaatgggctgatgaattagttaacatgcaggatgagatcgccgatataataatgaaagaagtaggtaagggctataaagacgccaaaaaagaagtcgttagaactgcggatttcataaggtacacgatagaggaagcattacacatgcatggtgaatccatgatgggcgatagttttccaggtggcaccaaaagcaaattggctatcatccaacgtgccccattgggtgttgtgttagcaattgcaccatttaattaccctgtgaatttatccgcggcaaagctagctcccgcgctgattatgggcaatgcggtgattttcaaaccggcaactcaaggtgccatatctggcataaaaatggttgaggcacttcataaagcgggattgcccaaaggcctagttaatgtggctacagggagaggaagtgccattggtgattatcttgtgggtcacgaagggattaacatggtaagttttacgggtggcactaataccggaaaacatttggcgaagactgccgcaatgatccctttggtacttgaacttggcggtaaagatccaggtattgttagggaggatgccgatctgcaggatgcagccaaccacattgtatctggagcttttagttactctggccaaagatgtaccgctataaaaagagttctggtccatgaaaatgtagctgacgaacttgttgacttactaaaggcccaggtcgctgaattatcagttggttctccagagaaagacagtactattgttagattgattgatgacaaatcagcggatttcgttcaaggtttggttgacgatgcagttgagaagggggcgaccatagtcataggtaataaaagagaaagaaacttaatatatccaacacttatagaccatgtgaaggaggagatgaaggtagcttgggaagagccttttggcccaatattgccaatcattagagcctcttcagatgagcaagctattgaaatcgcaaacaaatcggagttcgggttgcaagcaagtgtatttacaaaggacataaataaagctttcgccattgctaataagattgaaacaggttctgtgcagattaatggcagaacggagagaggtccagatcattttcccttcatcggtgtaaaagggtcaggcatgggagcacaggggataaggaagagtttggagtctatgactagagaaaaggttactgtgcttaatttcaaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 11 - DNA, 3850 bp, synthetic construct
  source 1..3850: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttatttgaaattaagcacagtaaccttttctctagtcatagactccaaactcttccttatcccctgtgctcccatgcctgacccttttacaccgatgaagggaaaatgatctggacctctctccgttctgccattaatctgcacagaacctgtttcaatcttattagcaatggcgaaagctttatttatgtcctttgtaaatacacttgcttgcaacccgaactccgatttgtttgcgatttcaatagcttgctcatctgaagaggctctaatgattggcaatattgggccaaaaggctcttcccaagctaccttcatctcctccttcacatggtctataagtgttggatatattaagtttctttctcttttattacctatgactatggtcgcccccttctcaactgcatcgtcaaccaaaccttgaacgaaatccgctgatttgtcatcaatcaatctaacaatagtactgtctttctctggagaaccaactgataattcagcgacctgggcctttagtaagtcaacaagttcgtcagctacattttcatggaccagaactctttttatagcggtacatctttggccagagtaactaaaagctccagatacaatgtggttggctgcatcctgcagatcggcatcctccctaacaatacctggatctttaccgccaagttcaagtaccaaagggatcattgcggcagtcttcgccaaatgttttccggtattagtgccacccgtaaaacttaccatgttaatcccttcgtgacccacaagataatcaccaatggcacttcctctccctgtagccacattaactaggcctttgggcaatcccgctttatgaagtgcctcaaccatttttatgccagatatggcaccttgagttgccggtttgaaaatcaccgcattgcccataatcagcgcgggagctagctttgccgcggataaattcacagggtaattaaatggtgcaattgctaacacaacacccaatggggcacgttggatgatagccaatttgcttttggtgccacctggaaaactatcgcccatcatggattcaccatgcatgtgtaatgcttcctctatcgtgtaccttatgaaatccgcagttctaacgacttcttttttggcgtctttatagcccttacctacttctttcattattatatcggcgatctcatcctgcatgttaactaattcatcagcccatttgtataagtacttggcacggtcttgtaaggatgcttttgcccatgacttttgcgcttcttttgccgaagctatagcttcatctacctccccacatgtaatggcttgcacttgtccaatcacctcatgcaaatatgggctggagatttcaatggtttcaccactagaagattccctccattcgccattaacatagaaattatatacattgacaaccatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 12 - protein, 478 aa, Bacillus thuringiensis
MVVNVYNFYVNGEWRESSSGETIEISSPYLHEVIGQVQAITCGEVDEAIASAKEAQKSWAKASLQDRAKYLYKWADELVNMQDEIADIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKAGLPKGLVNVATGRGSAIGDYLVGHEGINMVSFTGGTNTGKHLAKTAAMIPLVLELGGKDPGIVREDADLQDAANHIVSGAFSYSGQRCTAIKRVLVHENVADELVDLLKAQVAELSVGSPEKDSTIVRLIDDKSADFVQGLVDDAVEKGATIVIGNKRERNLIYPTLIDHVKEEMKVAWEEPFGPILPIIRASSDEQAIEIANKSEFGLQASVFTKDINKAFAIANKIETGSVQINGRTERGPDHFPFIGVKGSGMGAQGIRKSLESMTREKVTVLNFK

SEQ ID NO: 13 - DNA, 1437 bp, synthetic construct
  source 1..1437: mol_type = other DNA
atggttgtcaatgtatataatttctatgttaatggcgaatggagggaatcttctagtggtgaaaccattgaaatctccagcccatatttgcatgaggtgattggacaagtgcaagccattacatgtggggaggtagatgaagctatagcttcggcaaaagaagcgcaaaagtcatgggcaaaagcatccttacaagaccgtgccaagtacttatacaaatgggctgatgaattagttaacatgcaggatgagatcgccgatataataatgaaagaagtaggtaagggctataaagacgccaaaaaagaagtcgttagaactgcggatttcataaggtacacgatagaggaagcattacacatgcatggtgaatccatgatgggcgatagttttccaggtggcaccaaaagcaaattggctatcatccaacgtgccccattgggtgttgtgttagcaattgcaccatttaattaccctgtgaatttatccgcggcaaagctagctcccgcgctgattatgggcaatgcggtgattttcaaaccggcaactcaaggtgccatatctggcataaaaatggttgaggcacttcataaagcgggattgcccaaaggcctagttaatgtggctacagggagaggaagtgccattggtgattatcttgtgggtcacgaagggattaacatggtaagttttacgggtggcactaataccggaaaacatttggcgaagactgccgcaatgatccctttggtacttgaacttggcggtaaagatccaggtattgttagggaggatgccgatctgcaggatgcagccaaccacattgtatctggagcttttagttactctggccaaagatgtaccgctataaaaagagttctggtccatgaaaatgtagctgacgaacttgttgacttactaaaggcccaggtcgctgaattatcagttggttctccagagaaagacagtactattgttagattgattgatgacaaatcagcggatttcgttcaaggtttggttgacgatgcagttgagaagggggcgaccatagtcataggtaataaaagagaaagaaacttaatatatccaacacttatagaccatgtgaaggaggagatgaaggtagcttgggaagagccttttggcccaatattgccaatcattagagcctcttcagatgagcaagctattgaaatcgcaaacaaatcggagttcgggttgcaagcaagtgtatttacaaaggacataaataaagctttcgccattgctaataagattgaaacaggttctgtgcagattaatggcagaacggagagaggtccagatcattttcccttcatcggtgtaaaagggtcaggcatgggagcacaggggataaggaagagtttggagtctatgactagagaaaaggttactgtgcttaatttcaaataa

SEQ ID NO: 14 - DNA, 4088 bp, synthetic construct
  source 1..4088: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgaaagatgcaacaaagtctaaatctgaagtgcgtagagaaggcatgaggatcgagggggagaaagtcttcacagatgaagttgttgaagtgcgttatgcatataccgatgaagtgataggaacagtccctgctggcaaagccgaacacgcaagaaaagcctttgagattgctgcggcctataagcctaaactaaccagatacgaaaggcaacaaattttatttagagccgcagagttaataagaggaaggagagaagaaattgcacactggctagtactagagttaggcatttgcaagcaacacgcactatatgaaacaggtagatcttatgacgtcttcactttggctggccaattagcgatacaggacgacggacagattttttcttgtgatcttaccccacacgggaaagaccgtaagatttatacaaaaagagagccagttagagcaatctctgcaattaccccatttaatcaccctcttaacatggttgctcataagattgcacctgccatagctacaaataattgtgttgtttgtaagccaacagaacaaacgccacttactgccataacgttagcagatatcctttatgaggctggtttgccccctgaaatgtttcagatcgttacaggttggcctaaagatatcggagaagaaatgataacaaatgaaaatattgacattattacttttacgggtggcgtgagtgttggtaagttgatagcagcaaaagcaggttacaaaagacaagcacttgaattaggaggaaatgacccattgattgttcttaacgatctagacgatggagatttggacaaagccgctgccctggcagtggctggtgcaactggtaattccggtcaaagatgtaccgctgtcaagagaattcttgtacaggagtcagttgcggataggttcgtccctatggttttgaagaaagctaaagcaattaggtttggggacccaatggatccggagactcaattgggttgtgttatacatgcagaagcagctgcacttttcgaaaagagagtttatatggccgctgaacaaggagctaaagttttgtacgaccctggtagatccggtgcgctgttaccaccgatcgtcgtagatcacgttcctcatgattcagaattggttatggaagaaacctttggaccaattgttcctatcgtgagggtttctgacgatgatgcagaagttattagaatatccaattcaaccccatttggtttgtcatcaggtgtgtgcacgaacgattttagaagaatgcaaagttatattgaaggtcttaatgtcggcacagtgaatatttgggaacaacccggttatagaattgaaatgagtccttttggaggtatcaaagattcaggtaatggagtcaaagaaggcgtcttggaggcaatgaagttttttacaaatgtgaaaacttactctcttccctggcctagataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 15 - DNA, 3877 bp, synthetic construct
  source 1..3877: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttatctaggccagggaagagagtaagttttcacatttgtaaaaaacttcattgcctccaagacgccttctttgactccattacctgaatctttgatacctccaaaaggactcatttcaattctataaccgggttgttcccaaatattcactgtgccgacattaagaccttcaatataactttgcattcttctaaaatcgttcgtgcacacacctgatgacaaaccaaatggggttgaattggatattctaataacttctgcatcatcgtcagaaaccctcacgataggaacaattggtccaaaggtttcttccataaccaattctgaatcatgaggaacgtgatctacgacgatcggtggtaacagcgcaccggatctaccagggtcgtacaaaactttagctccttgttcagcggccatataaactctcttttcgaaaagtgcagctgcttctgcatgtataacacaacccaattgagtctccggatccattgggtccccaaacctaattgctttagctttcttcaaaaccatagggacgaacctatccgcaactgactcctgtacaagaattctcttgacagcggtacatctttgaccggaattaccagttgcaccagccactgccagggcagcggctttgtccaaatctccatcgtctagatcgttaagaacaatcaatgggtcatttcctcctaattcaagtgcttgtcttttgtaacctgcttttgctgctatcaacttaccaacactcacgccacccgtaaaagtaataatgtcaatattttcatttgttatcatttcttctccgatatctttaggccaacctgtaacgatctgaaacatttcagggggcaaaccagcctcataaaggatatctgctaacgttatggcagtaagtggcgtttgttctgttggcttacaaacaacacaattatttgtagctatggcaggtgcaatcttatgagcaaccatgttaagagggtgattaaatggggtaattgcagagattgctctaactggctctctttttgtataaatcttacggtctttcccgtgtggggtaagatcacaagaaaaaatctgtccgtcgtcctgtatcgctaattggccagccaaagtgaagacgtcataagatctacctgtttcatatagtgcgtgttgcttgcaaatgcctaactctagtactagccagtgtgcaatttcttctctccttcctcttattaactctgcggctctaaataaaatttgttgcctttcgtatctggttagtttaggcttataggccgcagcaatctcaaaggcttttcttgcgtgttcggctttgccagcagggactgttcctatcacttcatcggtatatgcataacgcacttcaacaacttcatctgtgaagactttctccccctcgatcctcatgccttctctacgcacttcagatttagactttgttgcatctttcatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 16 - protein, 487 aa, Defluviimonas aquaemixtae
MKDATKSKSEVRREGMRIEGEKVFTDEVVEVRYAYTDEVIGTVPAGKAEHARKAFEIAAAYKPKLTRYERQQILFRAAELIRGRREEIAHWLVLELGICKQHALYETGRSYDVFTLAGQLAIQDDGQIFSCDLTPHGKDRKIYTKREPVRAISAITPFNHPLNMVAHKIAPAIATNNCVVCKPTEQTPLTAITLADILYEAGLPPEMFQIVTGWPKDIGEEMITNENIDIITFTGGVSVGKLIAAKAGYKRQALELGGNDPLIVLNDLDDGDLDKAAALAVAGATGNSGQRCTAVKRILVQESVADRFVPMVLKKAKAIRFGDPMDPETQLGCVIHAEAAALFEKRVYMAAEQGAKVLYDPGRSGALLPPIVVDHVPHDSELVMEETFGPIVPIVRVSDDDAEVIRISNSTPFGLSSGVCTNDFRRMQSYIEGLNVGTVNIWEQPGYRIEMSPFGGIKDSGNGVKEGVLEAMKFFTNVKTYSLPWPR

SEQ ID NO: 17 - DNA, 1464 bp, synthetic construct
  source 1..1464: mol_type = other DNA
atgaaagatgcaacaaagtctaaatctgaagtgcgtagagaaggcatgaggatcgagggggagaaagtcttcacagatgaagttgttgaagtgcgttatgcatataccgatgaagtgataggaacagtccctgctggcaaagccgaacacgcaagaaaagcctttgagattgctgcggcctataagcctaaactaaccagatacgaaaggcaacaaattttatttagagccgcagagttaataagaggaaggagagaagaaattgcacactggctagtactagagttaggcatttgcaagcaacacgcactatatgaaacaggtagatcttatgacgtcttcactttggctggccaattagcgatacaggacgacggacagattttttcttgtgatcttaccccacacgggaaagaccgtaagatttatacaaaaagagagccagttagagcaatctctgcaattaccccatttaatcaccctcttaacatggttgctcataagattgcacctgccatagctacaaataattgtgttgtttgtaagccaacagaacaaacgccacttactgccataacgttagcagatatcctttatgaggctggtttgccccctgaaatgtttcagatcgttacaggttggcctaaagatatcggagaagaaatgataacaaatgaaaatattgacattattacttttacgggtggcgtgagtgttggtaagttgatagcagcaaaagcaggttacaaaagacaagcacttgaattaggaggaaatgacccattgattgttcttaacgatctagacgatggagatttggacaaagccgctgccctggcagtggctggtgcaactggtaattccggtcaaagatgtaccgctgtcaagagaattcttgtacaggagtcagttgcggataggttcgtccctatggttttgaagaaagctaaagcaattaggtttggggacccaatggatccggagactcaattgggttgtgttatacatgcagaagcagctgcacttttcgaaaagagagtttatatggccgctgaacaaggagctaaagttttgtacgaccctggtagatccggtgcgctgttaccaccgatcgtcgtagatcacgttcctcatgattcagaattggttatggaagaaacctttggaccaattgttcctatcgtgagggtttctgacgatgatgcagaagttattagaatatccaattcaaccccatttggtttgtcatcaggtgtgtgcacgaacgattttagaagaatgcaaagttatattgaaggtcttaatgtcggcacagtgaatatttgggaacaacccggttatagaattgaaatgagtccttttggaggtatcaaagattcaggtaatggagtcaaagaaggcgtcttggaggcaatgaagttttttacaaatgtgaaaacttactctcttccctggcctagataa

SEQ ID NO: 18 - DNA, 4358 bp, synthetic construct
  source 1..4358: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgaggcaaagaagcttagctacggctataggggcagcttttgaaagattgtctgaagccgtctgtgcttggcgtggtagatttgattctccaaggttagcttttgccggcttaccttttgcaagtgatgttagctctagacctggtgtcgccatggcttctgtagctacttctccgagagcaccagcttttaggggccctgctgcttctgcagatggattattaagaggtacatccttagaggacggcgttgccgaaaggattagagcagcaagagcggctcaggcaggatggggttcaagaccactgagagatcgtctgggcgtacttagaagattgaggttagcagttgtcgctaatccaagacatctggctgatgccgttccaagagataatttggctgaaacacttgctgcagaaatattgcctcttctagatgcttgtagattcttagaacatgaagctagggctgtgttgaaacccagaaccttgtccactgggtctagacctatgtggttgtggggtaacggtgtcacattgcgtagagatcccctgggcgccgtcttgattgtcggtccatccaattaccctttgatgttaccgggtgttcagattttacaagccgtcacagcaggtaatggggtattagttaaaccagctccaggttgctcaagaccaatagagatgttaattggtttaatggaatccgcgggtcttccaccaggtgtcgtgcaactgctgccggagagtcctgaagcagcggctgcggctatcagagcaggagttgataaagtttttttaacgggctctgcaccagcaggcagagcagtgagtagggacctgggagaaggaactgttccttctgtaatggaattgtctggatgtgatgcagttttcgtactagacgacgcagatccagaattagtaagcgattcacttttgtttggtctgactttgaatggatcacgtacttgcatggcaccaagacgtgtgttcgcttcacccgctatggtcgaacgtgtgctaggtaggttgagggacaagctaagactgaggtcaactgatgtaagtagaagaccacttgatgaaaagggtgccagaggagctggtgaagcagttagagacgcattaagaagtggtgcacaattagcagctggcacctttgatgcagacacacccaggcccctactgcgtgaaccagttgtactggaccatgtgacctctgacatgaccgtggctagaagtgatttgttcgcgccagtcttatcatttttgcgtgtttcttccgaagacgaagctctgagagaaaatgaaagatgcccgtatgcccttagtgccactgtttttggctctgacagaagatgcaggcaaattgccgagagaattcccgcgggttgtgtagttttaaatgacttaatagtacctacggctgatccaagagttccttttggtggaagaggtgcgagtggccatggggttactagaggtgccgctggtttagaagccatgactcacctgaaagccatcgttgctacaagaagatggtttaaaccacacctagagactcctacttcagctgatgcagatgtcttagaacaattgataagactggaacatgccgcctcccccttagaaaggttagctgcagtccctaggatgattagggctacgctggctcaatttagaattcgtagagctttgtcgcgtccatctcaagaagggcctcgttcctaacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 19 - DNA, 4147 bp, synthetic construct
  source 1..4147: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttaggaacgaggcccttcttgagatggacgcgacaaagctctacgaattctaaattgagccagcgtagccctaatcatcctagggactgcagctaacctttctaagggggaggcggcatgttccagtcttatcaattgttctaagacatctgcatcagctgaagtaggagtctctaggtgtggtttaaaccatcttcttgtagcaacgatggctttcaggtgagtcatggcttctaaaccagcggcacctctagtaaccccatggccactcgcacctcttccaccaaaaggaactcttggatcagccgtaggtactattaagtcatttaaaactacacaacccgcgggaattctctcggcaatttgcctgcatcttctgtcagagccaaaaacagtggcactaagggcatacgggcatctttcattttctctcagagcttcgtcttcggaagaaacacgcaaaaatgataagactggcgcgaacaaatcacttctagccacggtcatgtcagaggtcacatggtccagtacaactggttcacgcagtaggggcctgggtgtgtctgcatcaaaggtgccagctgctaattgtgcaccacttcttaatgcgtctctaactgcttcaccagctcctctggcacccttttcatcaagtggtcttctacttacatcagttgacctcagtcttagcttgtccctcaacctacctagcacacgttcgaccatagcgggtgaagcgaacacacgtcttggtgccatgcaagtacgtgatccattcaaagtcagaccaaacaaaagtgaatcgcttactaattctggatctgcgtcgtctagtacgaaaactgcatcacatccagacaattccattacagaaggaacagttccttctcccaggtccctactcactgctctgcctgctggtgcagagcccgttaaaaaaactttatcaactcctgctctgatagccgcagccgctgcttcaggactctccggcagcagttgcacgacacctggtggaagacccgcggattccattaaaccaattaacatctctattggtcttgagcaacctggagctggtttaactaataccccattacctgctgtgacggcttgtaaaatctgaacacccggtaacatcaaagggtaattggatggaccgacaatcaagacggcgcccaggggatctctacgcaatgtgacaccgttaccccacaaccacataggtctagacccagtggacaaggttctgggtttcaacacagccctagcttcatgttctaagaatctacaagcatctagaagaggcaatatttctgcagcaagtgtttcagccaaattatctcttggaacggcatcagccagatgtcttggattagcgacaactgctaacctcaatcttctaagtacgcccagacgatctctcagtggtcttgaaccccatcctgcctgagccgctcttgctgctctaatcctttcggcaacgccgtcctctaaggatgtacctcttaataatccatctgcagaagcagcagggcccctaaaagctggtgctctcggagaagtagctacagaagccatggcgacaccaggtctagagctaacatcacttgcaaaaggtaagccggcaaaagctaaccttggagaatcaaatctaccacgccaagcacagacggcttcagacaatctttcaaaagctgcccctatagccgtagctaagcttctttgcctcatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 20 - protein, 577 aa, Planctomyces sp.
MRQRSLATAIGAAFERLSEAVCAWRGRFDSPRLAFAGLPFASDVSSRPGVAMASVATSPRAPAFRGPAASADGLLRGTSLEDGVAERIRAARAAQAGWGSRPLRDRLGVLRRLRLAVVANPRHLADAVPRDNLAETLAAEILPLLDACRFLEHEARAVLKPRTLSTGSRPMWLWGNGVTLRRDPLGAVLIVGPSNYPLMLPGVQILQAVTAGNGVLVKPAPGCSRPIEMLIGLMESAGLPPGVVQLLPESPEAAAAAIRAGVDKVFLTGSAPAGRAVSRDLGEGTVPSVMELSGCDAVFVLDDADPELVSDSLLFGLTLNGSRTCMAPRRVFASPAMVERVLGRLRDKLRLRSTDVSRRPLDEKGARGAGEAVRDALRSGAQLAAGTFDADTPRPLLREPVVLDHVTSDMTVARSDLFAPVLSFLRVSSEDEALRENERCPYALSATVFGSDRRCRQIAERIPAGCVVLNDLIVPTADPRVPFGGRGASGHGVTRGAAGLEAMTHLKAIVATRRWFKPHLETPTSADADVLEQLIRLEHAASPLERLAAVPRMIRATLAQFRIRRALSRPSQEGPRS

SEQ ID NO: 21 - DNA, 1734 bp, synthetic construct
  source 1..1734: mol_type = other DNA
atgaggcaaagaagcttagctacggctataggggcagcttttgaaagattgtctgaagccgtctgtgcttggcgtggtagatttgattctccaaggttagcttttgccggcttaccttttgcaagtgatgttagctctagacctggtgtcgccatggcttctgtagctacttctccgagagcaccagcttttaggggccctgctgcttctgcagatggattattaagaggtacatccttagaggacggcgttgccgaaaggattagagcagcaagagcggctcaggcaggatggggttcaagaccactgagagatcgtctgggcgtacttagaagattgaggttagcagttgtcgctaatccaagacatctggctgatgccgttccaagagataatttggctgaaacacttgctgcagaaatattgcctcttctagatgcttgtagattcttagaacatgaagctagggctgtgttgaaacccagaaccttgtccactgggtctagacctatgtggttgtggggtaacggtgtcacattgcgtagagatcccctgggcgccgtcttgattgtcggtccatccaattaccctttgatgttaccgggtgttcagattttacaagccgtcacagcaggtaatggggtattagttaaaccagctccaggttgctcaagaccaatagagatgttaattggtttaatggaatccgcgggtcttccaccaggtgtcgtgcaactgctgccggagagtcctgaagcagcggctgcggctatcagagcaggagttgataaagtttttttaacgggctctgcaccagcaggcagagcagtgagtagggacctgggagaaggaactgttccttctgtaatggaattgtctggatgtgatgcagttttcgtactagacgacgcagatccagaattagtaagcgattcacttttgtttggtctgactttgaatggatcacgtacttgcatggcaccaagacgtgtgttcgcttcacccgctatggtcgaacgtgtgctaggtaggttgagggacaagctaagactgaggtcaactgatgtaagtagaagaccacttgatgaaaagggtgccagaggagctggtgaagcagttagagacgcattaagaagtggtgcacaattagcagctggcacctttgatgcagacacacccaggcccctactgcgtgaaccagttgtactggaccatgtgacctctgacatgaccgtggctagaagtgatttgttcgcgccagtcttatcatttttgcgtgtttcttccgaagacgaagctctgagagaaaatgaaagatgcccgtatgcccttagtgccactgtttttggctctgacagaagatgcaggcaaattgccgagagaattcccgcgggttgtgtagttttaaatgacttaatagtacctacggctgatccaagagttccttttggtggaagaggtgcgagtggccatggggttactagaggtgccgctggtttagaagccatgactcacctgaaagccatcgttgctacaagaagatggtttaaaccacacctagagactcctacttcagctgatgcagatgtcttagaacaattgataagactggaacatgccgcctcccccttagaaaggttagctgcagtccctaggatgattagggctacgctggctcaatttagaattcgtagagctttgtcgcgtccatctcaagaagggcctcgttcctaa

SEQ ID NO: 22 - DNA, 4079 bp, synthetic construct
  source 1..4079: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgacgacaaatgtcttgactttaacagcttacgaattttacgtgaatggtgagtggaaaaaatcaaagagtggtgaaacaattgatattcagagtccatacttaaatgaagttataggacaagtccaagctataaccaaggaagaagtcgatgaagcaatttacttcgcgcaacaggctcagaaatcatgggcagaaacctcgcttcaggaacgtgcaaattacttacataaatgggctgatgaactggtgaacatgcaggatgagattgctgaaattattatgaaagaagttggcaagtcgtacaaggatgcaaaaaatgaggtagttaggtcggcagattttatcagatacaccattgaggaagcattacatatgcatggagaaagtatgagaggtgattctttccctggaggatcccaaaacaagattgctgtgatccaaagggtacctttgggagttgttctagcaattccacctttcaattaccccgtcaatttagcggctgcaaaggttgcacccgctttaatggctggaaattccgtaatatttaagccagccacccagggtgccctttctgggacaaagatgatcgaagctttacacagggctgagcttcccaaaggtctggtcaacttggtaactggcaggggtagcgtgattggggattacttagttgaacataaagggattaacatgatctcattcaccggtggaacaactacaggatttcatttggctaagaaggctagcatggttcctcttgtattagagctaggaggcaaggatcctgggatagttcgtgaagacgctgatttatatgaagctgccaagcaaatcgtgtcaggagcattctcctattccggccagcgttgtacagctattaaaagagtgttggtgcacgagaacgcagctgatcaactagtagaattattaaaagaggagattgaaaaactatcggtcggatcaccggaaaatggatccactgttgtcccattgatcgacgaacaaagcgccgattttgtacaaattcttatcgctgacgcaataggcaagggagctgctgttattacgggcaataaacgtgagaggaacttgatataccctactttgctagactatgttacggaggatatgagagtggcttgggaggaaccatttgggccggtactgcctataattagagtctccagtgatgaggaagctatccaaatagctaacgaatcagagttcggattgcaggcttctattttcactaaagacattaacaaggcttttacaatcgccaataaattagaaacaggatcggttcaaattaacggtagaacggaaaggggacctgatcattttcctttcataggggtgaaggggtcgggtatgggagctcaaggaattagaaagtcattggaaagtatgactagggaaaaagttacagtgttaaacattgttcagtaacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 23 - DNA, 3868 bp, synthetic construct
  source 1..3868: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttactgaacaatgtttaacactgtaactttttccctagtcatactttccaatgactttctaattccttgagctcccatacccgaccccttcacccctatgaaaggaaaatgatcaggtcccctttccgttctaccgttaatttgaaccgatcctgtttctaatttattggcgattgtaaaagccttgttaatgtctttagtgaaaatagaagcctgcaatccgaactctgattcgttagctatttggatagcttcctcatcactggagactctaattataggcagtaccggcccaaatggttcctcccaagccactctcatatcctccgtaacatagtctagcaaagtagggtatatcaagttcctctcacgtttattgcccgtaataacagcagctcccttgcctattgcgtcagcgataagaatttgtacaaaatcggcgctttgttcgtcgatcaatgggacaacagtggatccattttccggtgatccgaccgatagtttttcaatctcctcttttaataattctactagttgatcagctgcgttctcgtgcaccaacactcttttaatagctgtacaacgctggccggaataggagaatgctcctgacacgatttgcttggcagcttcatataaatcagcgtcttcacgaactatcccaggatccttgcctcctagctctaatacaagaggaaccatgctagccttcttagccaaatgaaatcctgtagttgttccaccggtgaatgagatcatgttaatccctttatgttcaactaagtaatccccaatcacgctacccctgccagttaccaagttgaccagacctttgggaagctcagccctgtgtaaagcttcgatcatctttgtcccagaaagggcaccctgggtggctggcttaaatattacggaatttccagccattaaagcgggtgcaacctttgcagccgctaaattgacggggtaattgaaaggtggaattgctagaacaactcccaaaggtaccctttggatcacagcaatcttgttttgggatcctccagggaaagaatcacctctcatactttctccatgcatatgtaatgcttcctcaatggtgtatctgataaaatctgccgacctaactacctcattttttgcatccttgtacgacttgccaacttctttcataataatttcagcaatctcatcctgcatgttcaccagttcatcagcccatttatgtaagtaatttgcacgttcctgaagcgaggtttctgcccatgatttctgagcctgttgcgcgaagtaaattgcttcatcgacttcttccttggttatagcttggacttgtcctataacttcatttaagtatggactctgaatatcaattgtttcaccactctttgattttttccactcaccattcacgtaaaattcgtaagctgttaaagtcaagacatttgtcgtcatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 24 - protein, 484 aa, Bacillus abyssalis
MTTNVLTLTAYEFYVNGEWKKSKSGETIDIQSPYLNEVIGQVQAITKEEVDEAIYFAQQAQKSWAETSLQERANYLHKWADELVNMQDEIAEIIMKEVGKSYKDAKNEVVRSADFIRYTIEEALHMHGESMRGDSFPGGSQNKIAVIQRVPLGVVLAIPPFNYPVNLAAAKVAPALMAGNSVIFKPATQGALSGTKMIEALHRAELPKGLVNLVTGRGSVIGDYLVEHKGINMISFTGGTTTGFHLAKKASMVPLVLELGGKDPGIVREDADLYEAAKQIVSGAFSYSGQRCTAIKRVLVHENAADQLVELLKEEIEKLSVGSPENGSTVVPLIDEQSADFVQILIADAIGKGAAVITGNKRERNLIYPTLLDYVTEDMRVAWEEPFGPVLPIIRVSSDEEAIQIANESEFGLQASIFTKDINKAFTIANKLETGSVQINGRTERGPDHFPFIGVKGSGMGAQGIRKSLESMTREKVTVLNIVQ

SEQ ID NO: 25 - DNA, 1455 bp, synthetic construct
  source 1..1455: mol_type = other DNA
atgacgacaaatgtcttgactttaacagcttacgaattttacgtgaatggtgagtggaaaaaatcaaagagtggtgaaacaattgatattcagagtccatacttaaatgaagttataggacaagtccaagctataaccaaggaagaagtcgatgaagcaatttacttcgcgcaacaggctcagaaatcatgggcagaaacctcgcttcaggaacgtgcaaattacttacataaatgggctgatgaactggtgaacatgcaggatgagattgctgaaattattatgaaagaagttggcaagtcgtacaaggatgcaaaaaatgaggtagttaggtcggcagattttatcagatacaccattgaggaagcattacatatgcatggagaaagtatgagaggtgattctttccctggaggatcccaaaacaagattgctgtgatccaaagggtacctttgggagttgttctagcaattccacctttcaattaccccgtcaatttagcggctgcaaaggttgcacccgctttaatggctggaaattccgtaatatttaagccagccacccagggtgccctttctgggacaaagatgatcgaagctttacacagggctgagcttcccaaaggtctggtcaacttggtaactggcaggggtagcgtgattggggattacttagttgaacataaagggattaacatgatctcattcaccggtggaacaactacaggatttcatttggctaagaaggctagcatggttcctcttgtattagagctaggaggcaaggatcctgggatagttcgtgaagacgctgatttatatgaagctgccaagcaaatcgtgtcaggagcattctcctattccggccagcgttgtacagctattaaaagagtgttggtgcacgagaacgcagctgatcaactagtagaattattaaaagaggagattgaaaaactatcggtcggatcaccggaaaatggatccactgttgtcccattgatcgacgaacaaagcgccgattttgtacaaattcttatcgctgacgcaataggcaagggagctgctgttattacgggcaataaacgtgagaggaacttgatataccctactttgctagactatgttacggaggatatgagagtggcttgggaggaaccatttgggccggtactgcctataattagagtctccagtgatgaggaagctatccaaatagctaacgaatcagagttcggattgcaggcttctattttcactaaagacattaacaaggcttttacaatcgccaataaattagaaacaggatcggttcaaattaacggtagaacggaaaggggacctgatcattttcctttcataggggtgaaggggtcgggtatgggagctcaaggaattagaaagtcattggaaagtatgactagggaaaaagttacagtgttaaacattgttcagtaa

SEQ ID NO: 26 - DNA, 4052 bp, synthetic construct
  source 1..4052: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatggccaagcaatataagaacttggttaatggtgaatggaagttgtctgaaaacgagatcaccatttatgcacctgctactggtgaagaacttggcagcgtccccgccatgactcaagccgaagtagatgccgtttatgcatccgccaagaaggccttatcagactggagagctctgtcatatgttgaaagggcagcttatttgcataaggctgcagatattttggttcgtgacgcagagaagattggtgcaatcttatcaaaagaagtagcaaaaggtcataaagctgcagtttctgaagtgattagaacagctgaaataattaattacgccgcagaagaaggtcttaggatggaaggagaagttcttgaaggcggaagcttcgaagccgcatctaaaaaaaagatcgctattgtcagaagagaaccagtaggtctagtgctagcaatatctccttttaactaccctgtaaatttagcaggcagcaagatagcaccagctttaatagcaggcaatgtagtggctttgaaaccaccaacacagggctcaataagcggtctgctgttagccgaggcttttgctgaagctggtattcctgcaggtgtatttaatacaataactgggagaggttctgttattggtgactatattgttgagcacgaggctgtaaatttcataaattttactggttctactcctattggcgagggtatcggaaagctagcaggtatgaggcctatcatgttagagctgggtggaaaagattctgccatcgtattggaagacgctgatcttgctttggctgcaaaaaacattgtagctggagcatttggttacagtggtcaaagatgcaccgccgtgaagagagttctggttatggataaagtagcagaccaattagctgctgaaatcaagacattggttgaaaagttgtcagtgggtatgccagaagacgacgctgatattactccgcttattgatacaagtgccgccgatttcgtagaaggtcttattaaagacgccacggacaaaggtgcaacagcattaacagcgtttaatagagaaggaaatttaatctcgcctgtgttattcgatcatgtaactacagatatgagattggcctgggaggaaccttttgggcctgtcttaccaattattcgtgttactaccgttgaagaggcaataaaaatttctaatgaaagtgagtacgggttgcaagcttcaattttcactactaattttcctaaagcctttggcattgcagaacaattggaagtaggcactgtacacctgaataacaaaacccaaagaggcaccgataatttcccatttcttggtgctaaaaagtcaggtgctggagttcaaggtgtaaaatattccattgaagctatgacaacagtgaagagtgttgttttcgacatacaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 27 - DNA, 3841 bp, synthetic construct
  source 1..3841: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttattgtatgtcgaaaacaacactcttcactgttgtcatagcttcaatggaatattttacaccttgaactccagcacctgactttttagcaccaagaaatgggaaattatcggtgcctctttgggttttgttattcaggtgtacagtgcctacttccaattgttctgcaatgccaaaggctttaggaaaattagtagtgaaaattgaagcttgcaacccgtactcactttcattagaaatttttattgcctcttcaacggtagtaacacgaataattggtaagacaggcccaaaaggttcctcccaggccaatctcatatctgtagttacatgatcgaataacacaggcgagattaaatttccttctctattaaacgctgttaatgctgttgcacctttgtccgtggcgtctttaataagaccttctacgaaatcggcggcacttgtatcaataagcggagtaatatcagcgtcgtcttctggcatacccactgacaacttttcaaccaatgtcttgatttcagcagctaattggtctgctactttatccataaccagaactctcttcacggcggtgcatctttgaccactgtaaccaaatgctccagctacaatgttttttgcagccaaagcaagatcagcgtcttccaatacgatggcagaatcttttccacccagctctaacatgataggcctcatacctgctagctttccgataccctcgccaataggagtagaaccagtaaaatttatgaaatttacagcctcgtgctcaacaatatagtcaccaataacagaacctctcccagttattgtattaaatacacctgcaggaataccagcttcagcaaaagcctcggctaacagcagaccgcttattgagccctgtgttggtggtttcaaagccactacattgcctgctattaaagctggtgctatcttgctgcctgctaaatttacagggtagttaaaaggagatattgctagcactagacctactggttctcttctgacaatagcgatcttttttttagatgcggcttcgaagcttccgccttcaagaacttctccttccatcctaagaccttcttctgcggcgtaattaattatttcagctgttctaatcacttcagaaactgcagctttatgaccttttgctacttcttttgataagattgcaccaatcttctctgcgtcacgaaccaaaatatctgcagccttatgcaaataagctgccctttcaacatatgacagagctctccagtctgataaggccttcttggcggatgcataaacggcatctacttcggcttgagtcatggcggggacgctgccaagttcttcaccagtagcaggtgcataaatggtgatctcgttttcagacaacttccattcaccattaaccaagttcttatattgcttggccatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 28 - protein, 475 aa, Streptococcus pyogenes
MAKQYKNLVNGEWKLSENEITIYAPATGEELGSVPAMTQAEVDAVYASAKKALSDWRALSYVERAAYLHKAADILVRDAEKIGAILSKEVAKGHKAAVSEVIRTAEIINYAAEEGLRMEGEVLEGGSFEAASKKKIAIVRREPVGLVLAISPFNYPVNLAGSKIAPALIAGNVVALKPPTQGSISGLLLAEAFAEAGIPAGVFNTITGRGSVIGDYIVEHEAVNFINFTGSTPIGEGIGKLAGMRPIMLELGGKDSAIVLEDADLALAAKNIVAGAFGYSGQRCTAVKRVLVMDKVADQLAAEIKTLVEKLSVGMPEDDADITPLIDTSAADFVEGLIKDATDKGATALTAFNREGNLISPVLFDHVTTDMRLAWEEPFGPVLPIIRVTTVEEAIKISNESEYGLQASIFTTNFPKAFGIAEQLEVGTVHLNNKTQRGTDNFPFLGAKKSGAGVQGVKYSIEAMTTVKSVVFDIQ

SEQ ID NO: 29 - DNA, 1428 bp, synthetic construct
  source 1..1428: mol_type = other DNA
atggccaagcaatataagaacttggttaatggtgaatggaagttgtctgaaaacgagatcaccatttatgcacctgctactggtgaagaacttggcagcgtccccgccatgactcaagccgaagtagatgccgtttatgcatccgccaagaaggccttatcagactggagagctctgtcatatgttgaaagggcagcttatttgcataaggctgcagatattttggttcgtgacgcagagaagattggtgcaatcttatcaaaagaagtagcaaaaggtcataaagctgcagtttctgaagtgattagaacagctgaaataattaattacgccgcagaagaaggtcttaggatggaaggagaagttcttgaaggcggaagcttcgaagccgcatctaaaaaaaagatcgctattgtcagaagagaaccagtaggtctagtgctagcaatatctccttttaactaccctgtaaatttagcaggcagcaagatagcaccagctttaatagcaggcaatgtagtggctttgaaaccaccaacacagggctcaataagcggtctgctgttagccgaggcttttgctgaagctggtattcctgcaggtgtatttaatacaataactgggagaggttctgttattggtgactatattgttgagcacgaggctgtaaatttcataaattttactggttctactcctattggcgagggtatcggaaagctagcaggtatgaggcctatcatgttagagctgggtggaaaagattctgccatcgtattggaagacgctgatcttgctttggctgcaaaaaacattgtagctggagcatttggttacagtggtcaaagatgcaccgccgtgaagagagttctggttatggataaagtagcagaccaattagctgctgaaatcaagacattggttgaaaagttgtcagtgggtatgccagaagacgacgctgatattactccgcttattgatacaagtgccgccgatttcgtagaaggtcttattaaagacgccacggacaaaggtgcaacagcattaacagcgtttaatagagaaggaaatttaatctcgcctgtgttattcgatcatgtaactacagatatgagattggcctgggaggaaccttttgggcctgtcttaccaattattcgtgttactaccgttgaagaggcaataaaaatttctaatgaaagtgagtacgggttgcaagcttcaattttcactactaattttcctaaagcctttggcattgcagaacaattggaagtaggcactgtacacctgaataacaaaacccaaagaggcaccgataatttcccatttcttggtgctaaaaagtcaggtgctggagttcaaggtgtaaaatattccattgaagctatgacaacagtgaagagtgttgttttcgacatacaataa

SEQ ID NO: 30 - DNA, 4250 bp, synthetic construct
  source 1..4250: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgaccacggctcaaatattgggtaatcttttccctagtgccggtgatatccctgaaaaatatagattggacggtcagattgaacaaagagaatatttggttgatggagagctaaaaacttggccaggtgccttagcccaagtaagaagccctgtttacttggccggtgctaacggagatgaacaagtaatcttaggctccacgcctctgttggacgccgataccgcgcttaccgctttagatgctgcagtcagggcttacgatcgtggtcaaggtttgtggcctactatgagggtggctgaacgtatacaacatgtagaaacgtttttaggccacatgagagaacaaagggaagcggtggtaaaattgttaatgtgggaaattggaaagaacttgaaagattccgaaaaggaatttgatagaacatgcgattatatagttgatactgtgaacgcattaaaggaattagatagaagatcatcaaggttcgaattagaacaggatacactgggtcaaatccgtagggttccaatgggtgtagctctatgtatgggtccatacaactaccctctgaacgaaacattcactaccttaattcctgccctaattatgggtaatacagtggtttttaagccagctaagcttggagttttgttgattagaccgttgttggaagcatttcgtgattccttccccgctggagtgatcaatgttatctacggttccggtagggaaactgtatctgctctgatggcaagtggtaaaatcgatatattcgcttttataggaactaacaaagcagcctccgatttaaaaaaattgcaccctagaccacatagattgagggcagcactaggtctggacgctaaaaacccaggaatcattttacccgaagttgatttagataatgcagttaatgaggctgtcactggttctctttcattcaacggacagagatgcactgctttgaagattctttttgtacatgaggatgtggtcgaggcctttatcgacaaatttaacgcaaaattggcaacccttaaaccaggcatgccatgggactccggtgttgcattaactccgttgcctgaagctagtaaagtggactatctacattcacttgtagccgacgctgtttcgaaaggagccgccgttgtaaacccgaacggtggtgaagcgcgtgcttcattcttttatccagctgtcctgtaccctgtgactcctcaaatgagagtctatcaagaagagcagtttgggcctgtagttccaatcgttccgtatagacacttagacacggttatagattacgtgttggaatcagattttggccaacaattgagcatcttcggaactaatccagttgcggtcggtaggctggttgacacatttgcaaatcaggtaggaagaataaatctaaatgcacagtgtcagagggggccggatacgtatcccttcaacggccgtaaaaattccgccgagggaaccttgagtgtgcacgacgcgttaagagtcttttccataagaaccttagtagccactaagtttcaagaaactaataaagaactgatctctgatatcatctccggtagatcctcaagctttcttacgacagattacattttttaacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 31 - DNA, 4039 bp, synthetic construct
  source 1..4039: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttaaaaaatgtaatctgtcgtaagaaagcttgaggatctaccggagatgatatcagagatcagttctttattagtttcttgaaacttagtggctactaaggttcttatggaaaagactcttaacgcgtcgtgcacactcaaggttccctcggcggaatttttacggccgttgaagggatacgtatccggccccctctgacactgtgcatttagatttattcttcctacctgatttgcaaatgtgtcaaccagcctaccgaccgcaactggattagttccgaagatgctcaattgttggccaaaatctgattccaacacgtaatctataaccgtgtctaagtgtctatacggaacgattggaactacaggcccaaactgctcttcttgatagactctcatttgaggagtcacagggtacaggacagctggataaaagaatgaagcacgcgcttcaccaccgttcgggtttacaacggcggctcctttcgaaacagcgtcggctacaagtgaatgtagatagtccactttactagcttcaggcaacggagttaatgcaacaccggagtcccatggcatgcctggtttaagggttgccaattttgcgttaaatttgtcgataaaggcctcgaccacatcctcatgtacaaaaagaatcttcaaagcagtgcatctctgtccgttgaatgaaagagaaccagtgacagcctcattaactgcattatctaaatcaacttcgggtaaaatgattcctgggtttttagcgtccagacctagtgctgccctcaatctatgtggtctagggtgcaatttttttaaatcggaggctgctttgttagttcctataaaagcgaatatatcgattttaccacttgccatcagagcagatacagtttccctaccggaaccgtagataacattgatcactccagcggggaaggaatcacgaaatgcttccaacaacggtctaatcaacaaaactccaagcttagctggcttaaaaaccactgtattacccataattagggcaggaattaaggtagtgaatgtttcgttcagagggtagttgtatggacccatacatagagctacacccattggaaccctacggatttgacccagtgtatcctgttctaattcgaaccttgatgatcttctatctaattcctttaatgcgttcacagtatcaactatataatcgcatgttctatcaaattccttttcggaatctttcaagttctttccaatttcccacattaacaattttaccaccgcttccctttgttctctcatgtggcctaaaaacgtttctacatgttgtatacgttcagccaccctcatagtaggccacaaaccttgaccacgatcgtaagccctgactgcagcatctaaagcggtaagcgcggtatcggcgtccaacagaggcgtggagcctaagattacttgttcatctccgttagcaccggccaagtaaacagggcttcttacttgggctaaggcacctggccaagtttttagctctccatcaaccaaatattctctttgttcaatctgaccgtccaatctatatttttcagggatatcaccggcactagggaaaagattacccaatatttgagccgtggtcatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 32 - protein, 541 aa, Pseudomonas fluorescens
MTTAQILGNLFPSAGDIPEKYRLDGQIEQREYLVDGELKTWPGALAQVRSPVYLAGANGDEQVILGSTPLLDADTALTALDAAVRAYDRGQGLWPTMRVAERIQHVETFLGHMREQREAVVKLLMWEIGKNLKDSEKEFDRTCDYIVDTVNALKELDRRSSRFELEQDTLGQIRRVPMGVALCMGPYNYPLNETFTTLIPALIMGNTVVFKPAKLGVLLIRPLLEAFRDSFPAGVINVIYGSGRETVSALMASGKIDIFAFIGTNKAASDLKKLHPRPHRLRAALGLDAKNPGIILPEVDLDNAVNEAVTGSLSFNGQRCTALKILFVHEDVVEAFIDKFNAKLATLKPGMPWDSGVALTPLPEASKVDYLHSLVADAVSKGAAVVNPNGGEARASFFYPAVLYPVTPQMRVYQEEQFGPVVPIVPYRHLDTVIDYVLESDFGQQLSIFGTNPVAVGRLVDTFANQVGRINLNAQCQRGPDTYPFNGRKNSAEGTLSVHDALRVFSIRTLVATKFQETNKELISDIISGRSSSFLTTDYIF

SEQ ID NO: 33 - DNA, 1626 bp, synthetic construct
  source 1..1626: mol_type = other DNA
atgaccacggctcaaatattgggtaatcttttccctagtgccggtgatatccctgaaaaatatagattggacggtcagattgaacaaagagaatatttggttgatggagagctaaaaacttggccaggtgccttagcccaagtaagaagccctgtttacttggccggtgctaacggagatgaacaagtaatcttaggctccacgcctctgttggacgccgataccgcgcttaccgctttagatgctgcagtcagggcttacgatcgtggtcaaggtttgtggcctactatgagggtggctgaacgtatacaacatgtagaaacgtttttaggccacatgagagaacaaagggaagcggtggtaaaattgttaatgtgggaaattggaaagaacttgaaagattccgaaaaggaatttgatagaacatgcgattatatagttgatactgtgaacgcattaaaggaattagatagaagatcatcaaggttcgaattagaacaggatacactgggtcaaatccgtagggttccaatgggtgtagctctatgtatgggtccatacaactaccctctgaacgaaacattcactaccttaattcctgccctaattatgggtaatacagtggtttttaagccagctaagcttggagttttgttgattagaccgttgttggaagcatttcgtgattccttccccgctggagtgatcaatgttatctacggttccggtagggaaactgtatctgctctgatggcaagtggtaaaatcgatatattcgcttttataggaactaacaaagcagcctccgatttaaaaaaattgcaccctagaccacatagattgagggcagcactaggtctggacgctaaaaacccaggaatcattttacccgaagttgatttagataatgcagttaatgaggctgtcactggttctctttcattcaacggacagagatgcactgctttgaagattctttttgtacatgaggatgtggtcgaggcctttatcgacaaatttaacgcaaaattggcaacccttaaaccaggcatgccatgggactccggtgttgcattaactccgttgcctgaagctagtaaagtggactatctacattcacttgtagccgacgctgtttcgaaaggagccgccgttgtaaacccgaacggtggtgaagcgcgtgcttcattcttttatccagctgtcctgtaccctgtgactcctcaaatgagagtctatcaagaagagcagtttgggcctgtagttccaatcgttccgtatagacacttagacacggttatagattacgtgttggaatcagattttggccaacaattgagcatcttcggaactaatccagttgcggtcggtaggctggttgacacatttgcaaatcaggtaggaagaataaatctaaatgcacagtgtcagagggggccggatacgtatcccttcaacggccgtaaaaattccgccgagggaaccttgagtgtgcacgacgcgttaagagtcttttccataagaaccttagtagccactaagtttcaagaaactaataaagaactgatctctgatatcatctccggtagatcctcaagctttcttacgacagattacattttttaa

SEQ ID NO: 34 - DNA, 4076 bp, synthetic construct
  source 1..4076: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgaccaccactgttttaaatgttagtacatactccttctatcttaacggggagtggaaagaaagtaaatcaggtcagactattgatatcgtttctccatacagacacgatgttattggtaaagtgcaagccataactaaggaagaagtagatgaagctgtacatgccgcgcaaaaagcacaaaagggatgggctgaaaccagtttacaagaccgtgcaaaatatctttataaatgggcggacgaattagtaaagatgcaaagtgaaatagccgaaataattatgagagaagttggtaaatctctaaaggacgcgaggaatgaggttgtgagaactgctgacttcataaggtacacagtcgaggaagccttacatatgaatggggaaagcatgaaaggagatagcttccctgggggatcaaagtccaaggtggccattgttgaaagggttcccctgggggttgtgctagctatccctccttttaactatcctgttaacttaagcgttgcaaaattagcacctgccttaattagtggaaatgcagtgatttttaaaccagcaactcaaggcgcgatatctggtataaagatgatcgaggctctagatcaagctgacatacctcagggtttggttaatgtggtgacaggtagaggtagtgttataggggattacttggttgaacacaagggaataaatatgatctccttcactgggggtaccaacacgggcacgcaattagccaagaaagctgggatgatacctcttgttttggaattagggggtaaagatcctggcattgtttgtgaagacgccgatttacaagaggccgccaaacatatcatatctggtgctttttcatactctggccagagatgcacagctattaaaagagtcttagttcatgaaaaagtggctaatgagctagtagacattttgaaagaggaagtacataagttgagcgtcggttccccagaagatggctgcactatagttccattaatcgattccaaggctgctgattttatccaaatattaattgctgatgcgctggggaaaggtgctgaagtggttaccgggaacaaaagagaaggaaatttgatttaccctacccttttggataacgttaatgacactatgagagtggcatgggaagaaccgtttggaccggtattaccaataatcagagtcgcgtctgaccaggaggccattgacatcgcgaatgaatctgagtttggtttacaagcgtcggtgttcactaaagatattaacaaggccttttccattgcaaacaagattgaagtgggttccgttcagattaacggtagaactgaaagaggtcctgatcatttcccctttattggtgttaaaggttcaggaatgggcgcccaaggaatcagaaaatctattgaatcaatgacaagagaaaaggtcacagttttaaatctgcagtaacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 35 - DNA, 3865 bp, synthetic construct
  source 1..3865: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttactgcagatttaaaactgtgaccttttctcttgtcattgattcaatagattttctgattccttgggcgcccattcctgaacctttaacaccaataaaggggaaatgatcaggacctctttcagttctaccgttaatctgaacggaacccacttcaatcttgtttgcaatggaaaaggccttgttaatatctttagtgaacaccgacgcttgtaaaccaaactcagattcattcgcgatgtcaatggcctcctggtcagacgcgactctgattattggtaataccggtccaaacggttcttcccatgccactctcatagtgtcattaacgttatccaaaagggtagggtaaatcaaatttccttctcttttgttcccggtaaccacttcagcacctttccccagcgcatcagcaattaatatttggataaaatcagcagccttggaatcgattaatggaactatagtgcagccatcttctggggaaccgacgctcaacttatgtacttcctctttcaaaatgtctactagctcattagccactttttcatgaactaagactcttttaatagctgtgcatctctggccagagtatgaaaaagcaccagatatgatatgtttggcggcctcttgtaaatcggcgtcttcacaaacaatgccaggatctttaccccctaattccaaaacaagaggtatcatcccagctttcttggctaattgcgtgcccgtgttggtacccccagtgaaggagatcatatttattcccttgtgttcaaccaagtaatcccctataacactacctctacctgtcaccacattaaccaaaccctgaggtatgtcagcttgatctagagcctcgatcatctttataccagatatcgcgccttgagttgctggtttaaaaatcactgcatttccactaattaaggcaggtgctaattttgcaacgcttaagttaacaggatagttaaaaggagggatagctagcacaacccccaggggaaccctttcaacaatggccaccttggactttgatcccccagggaagctatctcctttcatgctttccccattcatatgtaaggcttcctcgactgtgtaccttatgaagtcagcagttctcacaacctcattcctcgcgtcctttagagatttaccaacttctctcataattatttcggctatttcactttgcatctttactaattcgtccgcccatttataaagatattttgcacggtcttgtaaactggtttcagcccatcccttttgtgctttttgcgcggcatgtacagcttcatctacttcttccttagttatggcttgcactttaccaataacatcgtgtctgtatggagaaacgatatcaatagtctgacctgatttactttctttccactccccgttaagatagaaggagtatgtactaacatttaaaacagtggtggtcatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 36 - protein, 483 aa, Bacillus megaterium
MTTTVLNVSTYSFYLNGEWKESKSGQTIDIVSPYRHDVIGKVQAITKEEVDEAVHAAQKAQKGWAETSLQDRAKYLYKWADELVKMQSEIAEIIMREVGKSLKDARNEVVRTADFIRYTVEEALHMNGESMKGDSFPGGSKSKVAIVERVPLGVVLAIPPFNYPVNLSVAKLAPALISGNAVIFKPATQGAISGIKMIEALDQADIPQGLVNVVTGRGSVIGDYLVEHKGINMISFTGGTNTGTQLAKKAGMIPLVLELGGKDPGIVCEDADLQEAAKHIISGAFSYSGQRCTAIKRVLVHEKVANELVDILKEEVHKLSVGSPEDGCTIVPLIDSKAADFIQILIADALGKGAEVVTGNKREGNLIYPTLLDNVNDTMRVAWEEPFGPVLPIIRVASDQEAIDIANESEFGLQASVFTKDINKAFSIANKIEVGSVQINGRTERGPDHFPFIGVKGSGMGAQGIRKSIESMTREKVTVLNLQ

SEQ ID NO: 37 - DNA, 1452 bp, synthetic construct
  source 1..1452: mol_type = other DNA
atgaccaccactgttttaaatgttagtacatactccttctatcttaacggggagtggaaagaaagtaaatcaggtcagactattgatatcgtttctccatacagacacgatgttattggtaaagtgcaagccataactaaggaagaagtagatgaagctgtacatgccgcgcaaaaagcacaaaagggatgggctgaaaccagtttacaagaccgtgcaaaatatctttataaatgggcggacgaattagtaaagatgcaaagtgaaatagccgaaataattatgagagaagttggtaaatctctaaaggacgcgaggaatgaggttgtgagaactgctgacttcataaggtacacagtcgaggaagccttacatatgaatggggaaagcatgaaaggagatagcttccctgggggatcaaagtccaaggtggccattgttgaaagggttcccctgggggttgtgctagctatccctccttttaactatcctgttaacttaagcgttgcaaaattagcacctgccttaattagtggaaatgcagtgatttttaaaccagcaactcaaggcgcgatatctggtataaagatgatcgaggctctagatcaagctgacatacctcagggtttggttaatgtggtgacaggtagaggtagtgttataggggattacttggttgaacacaagggaataaatatgatctccttcactgggggtaccaacacgggcacgcaattagccaagaaagctgggatgatacctcttgttttggaattagggggtaaagatcctggcattgtttgtgaagacgccgatttacaagaggccgccaaacatatcatatctggtgctttttcatactctggccagagatgcacagctattaaaagagtcttagttcatgaaaaagtggctaatgagctagtagacattttgaaagaggaagtacataagttgagcgtcggttccccagaagatggctgcactatagttccattaatcgattccaaggctgctgattttatccaaatattaattgctgatgcgctggggaaaggtgctgaagtggttaccgggaacaaaagagaaggaaatttgatttaccctacccttttggataacgttaatgacactatgagagtggcatgggaagaaccgtttggaccggtattaccaataatcagagtcgcgtctgaccaggaggccattgacatcgcgaatgaatctgagtttggtttacaagcgtcggtgttcactaaagatattaacaaggccttttccattgcaaacaagattgaagtgggttccgttcagattaacggtagaactgaaagaggtcctgatcatttcccctttattggtgttaaaggttcaggaatgggcgcccaaggaatcagaaaatctattgaatcaatgacaagagaaaaggtcacagttttaaatctgcagtaa

SEQ ID NO: 38 - DNA, 4085 bp, synthetic construct
  source 1..4085: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgttcaactgtataaaaagtgaaaataatgtgttcaaaaatttgattaatgggcaatggttaagtagcaaggaagataatttcattgaaatcaattcaccgatcgataactcaatcgtgggcaaaattcccgctatgacaaaagaagaagtcgattttgtgattagtactgctaagcgtgcgcagaaagactggtcaaaagtcccaatcaatgaacgtgcagagattttatataaagctgctgatattttagtcaagaatattgatgaattggccgaaataatggtgcgtgagattggcaaggacaggaaaagttcacgtagtgaaattacaaggacagctgactttataaggtttaccgccgacactgctaaaaacttgtccggtgaaagcatcccaggtgacacctttccaggttacaagcgtaacaaaatctcggttgtcagtcgtgaaccgttaggtgtggtgttggcaatttctcctttcaactatcccataaacctggcggcatctaaaatcgctccggctatcatagtcggtaactctgttgttttaaagccagctacccaaggctctttgtgtggcctatatcttgctaaagtgttccaagaggcaggagttccaaatggtgttttgaatactataacaggaaaaggttcggaaattggcgattatgttgtaactcacaaggacatcaattttataaactttactggtagtactgaagttggcactcgtataagtaaaattacatcaatggttccacttttgatggaactaggcgggaaggatgctgctattgtgttagaagatgctgatttggaattagctgcgactaatatagttgctggcggttattcttattcatcccaaagatgcactgcggtcaaaagaatacttgttattgacaaagtcgcagatgaactagttgcaaaggttaaagaaaagatatccaaattgaaagttggaaatccacttgttgacgatgtcgatattgtccctttgataaataaaaaagcagctgattatgtatcagaactaattgaagatgcaaagaataaaggcgccgatttagtggctggcggcaaccgtgatggaaatataatttttccaactttatttgataatgtaactactgacatgaggattgcatgggaagaacctttcggacctgttttaccgatcattagagttaaaaacaaggacgaagcaattgagattgctaacaaaagcgaatacggtctgcagtctgccgtattcacaaaaaacatagatgatgctttttacattgccgacaggttggaagttggtacggttcaagtgaacggtaaaactgagagaggcccagatcacttccccttcttaggtgtgaaagctagcggtataggcacacaaggcataagatattcaatcgaatcaatgtcgcgtcctaaagcaactgtgattaatttgttgaagtaacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 39 - DNA, 3874 bp, synthetic construct
  source 1..3874: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttacttcaacaaattaatcacagttgctttaggacgcgacattgattcgattgaatatcttatgccttgtgtgcctataccgctagctttcacacctaagaaggggaagtgatctgggcctctctcagttttaccgttcacttgaaccgtaccaacttccaacctgtcggcaatgtaaaaagcatcatctatgttttttgtgaatacggcagactgcagaccgtattcgcttttgttagcaatctcaattgcttcgtccttgtttttaactctaatgatcggtaaaacaggtccgaaaggttcttcccatgcaatcctcatgtcagtagttacattatcaaataaagttggaaaaattatatttccatcacggttgccgccagccactaaatcggcgcctttattctttgcatcttcaattagttctgatacataatcagctgcttttttatttatcaaagggacaatatcgacatcgtcaacaagtggatttccaactttcaatttggatatcttttctttaacctttgcaactagttcatctgcgactttgtcaataacaagtattcttttgaccgcagtgcatctttgggatgaataagaataaccgccagcaactatattagtcgcagctaattccaaatcagcatcttctaacacaatagcagcatccttcccgcctagttccatcaaaagtggaaccattgatgtaattttacttatacgagtgccaacttcagtactaccagtaaagtttataaaattgatgtccttgtgagttacaacataatcgccaatttccgaaccttttcctgttatagtattcaaaacaccatttggaactcctgcctcttggaacactttagcaagatataggccacacaaagagccttgggtagctggctttaaaacaacagagttaccgactatgatagccggagcgattttagatgccgccaggtttatgggatagttgaaaggagaaattgccaacaccacacctaacggttcacgactgacaaccgagattttgttacgcttgtaacctggaaaggtgtcacctgggatgctttcaccggacaagtttttagcagtgtcggcggtaaaccttataaagtcagctgtccttgtaatttcactacgtgaacttttcctgtccttgccaatctcacgcaccattatttcggccaattcatcaatattcttgactaaaatatcagcagctttatataaaatctctgcacgttcattgattgggacttttgaccagtctttctgcgcacgcttagcagtactaatcacaaaatcgacttcttcttttgtcatagcgggaattttgcccacgattgagttatcgatcggtgaattgatttcaatgaaattatcttccttgctacttaaccattgcccattaatcaaatttttgaacacattattttcactttttatacagttgaacatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 40 - protein, 486 aa, Clostridium saccharoperbutylacetonicum
MFNCIKSENNVFKNLINGQWLSSKEDNFIEINSPIDNSIVGKIPAMTKEEVDFVISTAKRAQKDWSKVPINERAEILYKAADILVKNIDELAEIMVREIGKDRKSSRSEITRTADFIRFTADTAKNLSGESIPGDTFPGYKRNKISVVSREPLGVVLAISPFNYPINLAASKIAPAIIVGNSVVLKPATQGSLCGLYLAKVFQEAGVPNGVLNTITGKGSEIGDYVVTHKDINFINFTGSTEVGTRISKITSMVPLLMELGGKDAAIVLEDADLELAATNIVAGGYSYSSQRCTAVKRILVIDKVADELVAKVKEKISKLKVGNPLVDDVDIVPLINKKAADYVSELIEDAKNKGADLVAGGNRDGNIIFPTLFDNVTTDMRIAWEEPFGPVLPIIRVKNKDEAIEIANKSEYGLQSAVFTKNIDDAFYIADRLEVGTVQVNGKTERGPDHFPFLGVKASGIGTQGIRYSIESMSRPKATVINLLK

SEQ ID NO: 41 - DNA, 1461 bp, synthetic construct
  source 1..1461: mol_type = other DNA
atgttcaactgtataaaaagtgaaaataatgtgttcaaaaatttgattaatgggcaatggttaagtagcaaggaagataatttcattgaaatcaattcaccgatcgataactcaatcgtgggcaaaattcccgctatgacaaaagaagaagtcgattttgtgattagtactgctaagcgtgcgcagaaagactggtcaaaagtcccaatcaatgaacgtgcagagattttatataaagctgctgatattttagtcaagaatattgatgaattggccgaaataatggtgcgtgagattggcaaggacaggaaaagttcacgtagtgaaattacaaggacagctgactttataaggtttaccgccgacactgctaaaaacttgtccggtgaaagcatcccaggtgacacctttccaggttacaagcgtaacaaaatctcggttgtcagtcgtgaaccgttaggtgtggtgttggcaatttctcctttcaactatcccataaacctggcggcatctaaaatcgctccggctatcatagtcggtaactctgttgttttaaagccagctacccaaggctctttgtgtggcctatatcttgctaaagtgttccaagaggcaggagttccaaatggtgttttgaatactataacaggaaaaggttcggaaattggcgattatgttgtaactcacaaggacatcaattttataaactttactggtagtactgaagttggcactcgtataagtaaaattacatcaatggttccacttttgatggaactaggcgggaaggatgctgctattgtgttagaagatgctgatttggaattagctgcgactaatatagttgctggcggttattcttattcatcccaaagatgcactgcggtcaaaagaatacttgttattgacaaagtcgcagatgaactagttgcaaaggttaaagaaaagatatccaaattgaaagttggaaatccacttgttgacgatgtcgatattgtccctttgataaataaaaaagcagctgattatgtatcagaactaattgaagatgcaaagaataaaggcgccgatttagtggctggcggcaaccgtgatggaaatataatttttccaactttatttgataatgtaactactgacatgaggattgcatgggaagaacctttcggacctgttttaccgatcattagagttaaaaacaaggacgaagcaattgagattgctaacaaaagcgaatacggtctgcagtctgccgtattcacaaaaaacatagatgatgctttttacattgccgacaggttggaagttggtacggttcaagtgaacggtaaaactgagagaggcccagatcacttccccttcttaggtgtgaaagctagcggtataggcacacaaggcataagatattcaatcgaatcaatgtcgcgtcctaaagcaactgtgattaatttgttgaagtaa

SEQ ID NO: 42 - DNA, 4073 bp, synthetic construct
  source 1..4073: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgaccgcgactgaggaaatcagacatgaagcgatgagaattggtggtgaaaaagtcttcactgacgatgtcgttgaagtacgttatccttatactgacgaagtagttgccactgttcctgcagggacagcagaacatgccgcccaagcatttgcaatagctgctgcatacaagccatccttgactcgttacgaaagacaaaaaattttgttcagagcggcggaactaatcagggaaaggagagattcaattgcacattggcttacgctggagttgggcatttgccaacaacatgcactgtacgagacaggtaggtcttatgatgtattcatgctggcaggtcaggctgccattcaagatgatggtcaaattttctcatgcgatcttacacctcatggaaaaccaagaaagattttcaccaagagggaaccagtaaggactatatcggccattacaccattcaatcatccgcttaacatggtggcccacaagattgctccggcaattgcaaccaacaattgtgtggtttgtaagcccacagagttaacacccttgacagctattactttggcagacatattatacgaagcagggctgccagtcgagatgttccaaattgttaccggttggccaaaagatattggtgacgaaatggtgacgaatgaaaacatcgatatagtgacctttacaggaggagtgccagttggtaagatgattgctcaaaaggcaggttataagagagctgctttggaattaggaggaaatgatcctctaataatccttaacgacttgagtgatgcagatttggagaaggctgcgactattgccgttgctggcgccaccggtaattctggccaaaggtgtacggccgttaaaagaatccttgtacaaaattcagttgcggataagttcgtaccattagttttagaaaaagctaaggcaatcagattcggagatcctatggatcccgacacccaactaggatgtgttatacatgctaaagctgccgaattattcgagaaaagggtatatatggcagaagctgaaggcgccgaaatactgtatcatcctggtagaaatggtgcacttttgcctccaatcgtaatcgataaagtaccacattcctcagagttggttatggaagaaaccttcggccccattgtacccattgttagagtgccagacgatgatgatgctgtgatggaaatcagtaactcaactgaattcgggttaagttccggtgtgtgcacgaacgacttcacaagaatgcaagctttcattgagggtcttgatgtcggaacggtaaacatctgggaacagcccggttacaggatcgaaatgtctccatttggcggtatcaaagattcaggtaatggcgttaaagaaggtgttctagaggcgatgaaattcttcactaatgttaagacatattcattgccatggccataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 43 - DNA, 3862 bp, synthetic construct
  source 1..3862: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttatggccatggcaatgaatatgtcttaacattagtgaagaatttcatcgcctctagaacaccttctttaacgccattacctgaatctttgataccgccaaatggagacatttcgatcctgtaaccgggctgttcccagatgtttaccgttccgacatcaagaccctcaatgaaagcttgcattcttgtgaagtcgttcgtgcacacaccggaacttaacccgaattcagttgagttactgatttccatcacagcatcatcatcgtctggcactctaacaatgggtacaatggggccgaaggtttcttccataaccaactctgaggaatgtggtactttatcgattacgattggaggcaaaagtgcaccatttctaccaggatgatacagtatttcggcgccttcagcttctgccatatatacccttttctcgaataattcggcagctttagcatgtataacacatcctagttgggtgtcgggatccataggatctccgaatctgattgccttagctttttctaaaactaatggtacgaacttatccgcaactgaattttgtacaaggattcttttaacggccgtacacctttggccagaattaccggtggcgccagcaacggcaatagtcgcagccttctccaaatctgcatcactcaagtcgttaaggattattagaggatcatttcctcctaattccaaagcagctctcttataacctgccttttgagcaatcatcttaccaactggcactcctcctgtaaaggtcactatatcgatgttttcattcgtcaccatttcgtcaccaatatcttttggccaaccggtaacaatttggaacatctcgactggcagccctgcttcgtataatatgtctgccaaagtaatagctgtcaagggtgttaactctgtgggcttacaaaccacacaattgttggttgcaattgccggagcaatcttgtgggccaccatgttaagcggatgattgaatggtgtaatggccgatatagtccttactggttccctcttggtgaaaatctttcttggttttccatgaggtgtaagatcgcatgagaaaatttgaccatcatcttgaatggcagcctgacctgccagcatgaatacatcataagacctacctgtctcgtacagtgcatgttgttggcaaatgcccaactccagcgtaagccaatgtgcaattgaatctctcctttccctgattagttccgccgctctgaacaaaattttttgtctttcgtaacgagtcaaggatggcttgtatgcagcagctattgcaaatgcttgggcggcatgttctgctgtccctgcaggaacagtggcaactacttcgtcagtataaggataacgtacttcaacgacatcgtcagtgaagactttttcaccaccaattctcatcgcttcatgtctgatttcctcagtcgcggtcatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 44 - protein, 482 aa, Rhodobacteraceae bacterium
MTATEEIRHEAMRIGGEKVFTDDVVEVRYPYTDEVVATVPAGTAEHAAQAFAIAAAYKPSLTRYERQKILFRAAELIRERRDSIAHWLTLELGICQQHALYETGRSYDVFMLAGQAAIQDDGQIFSCDLTPHGKPRKIFTKREPVRTISAITPFNHPLNMVAHKIAPAIATNNCVVCKPTELTPLTAITLADILYEAGLPVEMFQIVTGWPKDIGDEMVTNENIDIVTFTGGVPVGKMIAQKAGYKRAALELGGNDPLIILNDLSDADLEKAATIAVAGATGNSGQRCTAVKRILVQNSVADKFVPLVLEKAKAIRFGDPMDPDTQLGCVIHAKAAELFEKRVYMAEAEGAEILYHPGRNGALLPPIVIDKVPHSSELVMEETFGPIVPIVRVPDDDDAVMEISNSTEFGLSSGVCTNDFTRMQAFIEGLDVGTVNIWEQPGYRIEMSPFGGIKDSGNGVKEGVLEAMKFFTNVKTYSLPWP

SEQ ID NO: 45 - DNA, 1449 bp, synthetic construct
  source 1..1449: mol_type = other DNA
atgaccgcgactgaggaaatcagacatgaagcgatgagaattggtggtgaaaaagtcttcactgacgatgtcgttgaagtacgttatccttatactgacgaagtagttgccactgttcctgcagggacagcagaacatgccgcccaagcatttgcaatagctgctgcatacaagccatccttgactcgttacgaaagacaaaaaattttgttcagagcggcggaactaatcagggaaaggagagattcaattgcacattggcttacgctggagttgggcatttgccaacaacatgcactgtacgagacaggtaggtcttatgatgtattcatgctggcaggtcaggctgccattcaagatgatggtcaaattttctcatgcgatcttacacctcatggaaaaccaagaaagattttcaccaagagggaaccagtaaggactatatcggccattacaccattcaatcatccgcttaacatggtggcccacaagattgctccggcaattgcaaccaacaattgtgtggtttgtaagcccacagagttaacacccttgacagctattactttggcagacatattatacgaagcagggctgccagtcgagatgttccaaattgttaccggttggccaaaagatattggtgacgaaatggtgacgaatgaaaacatcgatatagtgacctttacaggaggagtgccagttggtaagatgattgctcaaaaggcaggttataagagagctgctttggaattaggaggaaatgatcctctaataatccttaacgacttgagtgatgcagatttggagaaggctgcgactattgccgttgctggcgccaccggtaattctggccaaaggtgtacggccgttaaaagaatccttgtacaaaattcagttgcggataagttcgtaccattagttttagaaaaagctaaggcaatcagattcggagatcctatggatcccgacacccaactaggatgtgttatacatgctaaagctgccgaattattcgagaaaagggtatatatggcagaagctgaaggcgccgaaatactgtatcatcctggtagaaatggtgcacttttgcctccaatcgtaatcgataaagtaccacattcctcagagttggttatggaagaaaccttcggccccattgtacccattgttagagtgccagacgatgatgatgctgtgatggaaatcagtaactcaactgaattcgggttaagttccggtgtgtgcacgaacgacttcacaagaatgcaagctttcattgagggtcttgatgtcggaacggtaaacatctgggaacagcccggttacaggatcgaaatgtctccatttggcggtatcaaagattcaggtaatggcgttaaagaaggtgttctagaggcgatgaaattcttcactaatgttaagacatattcattgccatggccataa

SEQ ID NO: 46 - DNA, 4079 bp, synthetic construct
  source 1..4079: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgactacaaaagcagcgccagcacacatgcaattaccctactacataaatggtgaatggagaaaaagcagctcaaatgaaaccgtcaaaatctatagcccatataaggacggtgtggtaggggaagtccaggcaatcaccagggaagaagtggatgaggctataaaattagctgcagaagctcaaaaggcttgggccgaacttagtattagagagagggctacgtacttatacagatggatcgacgaattattagcgatgaaagatgaaatagcagacattatgatgcaagaagtaggtaaaacttttgcagacgcccaaaaggaagttgaaagaagcgcagatctaataagatataatatagaagaggccattcacatgtataatgagtccatgtctggcgaaaactttccaggcggctcgaaacaaaagatagcaatcgtggagagagtccccctaggagttgttctagccatttctccgtttaattatcctgtcaacttatcatcggcaaaaattgcacccgctttgatgatgggtaatacagtgattttcaaaccggctacacaaggcgcactgtctggcattaagatgatagaggctttgcataaggctggcattcctaagaacatcgttaacgtagtgacaggtagaggttccgttataggtgactatttggtcgagcacaagggcattaacatgataactttcacgggcggtacagctactggttatcatatagcttctaaggctaaaatggtcccattagttatggaactaggggggaaggatccagctattgtcctaccagatgctgacttaaagcttgccacaggccagattatcagcggtgcctattcttattcaggacaaagatgtaccgccataaagagagttttagtccaagacgaagtggcagatcaactaacagatatgttagcggccgaagtagctaagctaagtattggttcgccggaagataaagctactgttgtccctgttatcgacactaaatctgcagactatatccagggtttgatagatgacgccattgagaaaggtgcaaaagtattaacaggtaataaaagagaggggaatttgatccacccaaccttattagctgatgtgactttagatatgagagttgcttgggaagaaccgttcggtccggttttgccaataattaggataaaggacgccgaagaagccatcgaaatagctaacaaatctgagttcggtttgcaagcgtctgttttcacaaacgacataaataaagcattccatttagcgaagaaattggaaaccggatcggtacagatcaacggcaggacagagcgtggaccggaccactttccatttataggtgttaagggttcgggtgttggtgttcaaggggtccccaagagtttgcaatcaatgacccgtgacaaagttacggttttgaatttgcaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 47 - DNA, 3868 bp, synthetic construct
  source 1..3868: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttattgcaaattcaaaaccgtaactttgtcacgggtcattgattgcaaactcttggggaccccttgaacaccaacacccgaacccttaacacctataaatggaaagtggtccggtccacgctctgtcctgccgttgatctgtaccgatccggtttccaatttcttcgctaaatggaatgctttatttatgtcgtttgtgaaaacagacgcttgcaaaccgaactcagatttgttagctatttcgatggcttcttcggcgtcctttatcctaattattggcaaaaccggaccgaacggttcttcccaagcaactctcatatctaaagtcacatcagctaataaggttgggtggatcaaattcccctctcttttattacctgttaatacttttgcacctttctcaatggcgtcatctatcaaaccctggatatagtctgcagatttagtgtcgataacagggacaacagtagctttatcttccggcgaaccaatacttagcttagctacttcggccgctaacatatctgttagttgatctgccacttcgtcttggactaaaactctctttatggcggtacatctttgtcctgaataagaataggcaccgctgataatctggcctgtggcaagctttaagtcagcatctggtaggacaatagctggatccttcccccctagttccataactaatgggaccattttagccttagaagctatatgataaccagtagctgtaccgcccgtgaaagttatcatgttaatgcccttgtgctcgaccaaatagtcacctataacggaacctctacctgtcactacgttaacgatgttcttaggaatgccagccttatgcaaagcctctatcatcttaatgccagacagtgcgccttgtgtagccggtttgaaaatcactgtattacccatcatcaaagcgggtgcaatttttgccgatgataagttgacaggataattaaacggagaaatggctagaacaactcctagggggactctctccacgattgctatcttttgtttcgagccgcctggaaagttttcgccagacatggactcattatacatgtgaatggcctcttctatattatatcttattagatctgcgcttctttcaacttccttttgggcgtctgcaaaagttttacctacttcttgcatcataatgtctgctatttcatctttcatcgctaataattcgtcgatccatctgtataagtacgtagccctctctctaatactaagttcggcccaagccttttgagcttctgcagctaattttatagcctcatccacttcttccctggtgattgcctggacttcccctaccacaccgtccttatatgggctatagattttgacggtttcatttgagctgctttttctccattcaccatttatgtagtagggtaattgcatgtgtgctggcgctgcttttgtagtcatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 48 - protein, 484 aa, Brevibacillus laterosporus
MTTKAAPAHMQLPYYINGEWRKSSSNETVKIYSPYKDGVVGEVQAITREEVDEAIKLAAEAQKAWAELSIRERATYLYRWIDELLAMKDEIADIMMQEVGKTFADAQKEVERSADLIRYNIEEAIHMYNESMSGENFPGGSKQKIAIVERVPLGVVLAISPFNYPVNLSSAKIAPALMMGNTVIFKPATQGALSGIKMIEALHKAGIPKNIVNVVTGRGSVIGDYLVEHKGINMITFTGGTATGYHIASKAKMVPLVMELGGKDPAIVLPDADLKLATGQIISGAYSYSGQRCTAIKRVLVQDEVADQLTDMLAAEVAKLSIGSPEDKATVVPVIDTKSADYIQGLIDDAIEKGAKVLTGNKREGNLIHPTLLADVTLDMRVAWEEPFGPVLPIIRIKDAEEAIEIANKSEFGLQASVFTNDINKAFHLAKKLETGSVQINGRTERGPDHFPFIGVKGSGVGVQGVPKSLQSMTRDKVTVLNLQ

SEQ ID NO: 49 - DNA, 1455 bp, synthetic construct
  source 1..1455: mol_type = other DNA
atgactacaaaagcagcgccagcacacatgcaattaccctactacataaatggtgaatggagaaaaagcagctcaaatgaaaccgtcaaaatctatagcccatataaggacggtgtggtaggggaagtccaggcaatcaccagggaagaagtggatgaggctataaaattagctgcagaagctcaaaaggcttgggccgaacttagtattagagagagggctacgtacttatacagatggatcgacgaattattagcgatgaaagatgaaatagcagacattatgatgcaagaagtaggtaaaacttttgcagacgcccaaaaggaagttgaaagaagcgcagatctaataagatataatatagaagaggccattcacatgtataatgagtccatgtctggcgaaaactttccaggcggctcgaaacaaaagatagcaatcgtggagagagtccccctaggagttgttctagccatttctccgtttaattatcctgtcaacttatcatcggcaaaaattgcacccgctttgatgatgggtaatacagtgattttcaaaccggctacacaaggcgcactgtctggcattaagatgatagaggctttgcataaggctggcattcctaagaacatcgttaacgtagtgacaggtagaggttccgttataggtgactatttggtcgagcacaagggcattaacatgataactttcacgggcggtacagctactggttatcatatagcttctaaggctaaaatggtcccattagttatggaactaggggggaaggatccagctattgtcctaccagatgctgacttaaagcttgccacaggccagattatcagcggtgcctattcttattcaggacaaagatgtaccgccataaagagagttttagtccaagacgaagtggcagatcaactaacagatatgttagcggccgaagtagctaagctaagtattggttcgccggaagataaagctactgttgtccctgttatcgacactaaatctgcagactatatccagggtttgatagatgacgccattgagaaaggtgcaaaagtattaacaggtaataaaagagaggggaatttgatccacccaaccttattagctgatgtgactttagatatgagagttgcttgggaagaaccgttcggtccggttttgccaataattaggataaaggacgccgaagaagccatcgaaatagctaacaaatctgagttcggtttgcaagcgtctgttttcacaaacgacataaataaagcattccatttagcgaagaaattggaaaccggatcggtacagatcaacggcaggacagagcgtggaccggaccactttccatttataggtgttaagggttcgggtgttggtgttcaaggggtccccaagagtttgcaatcaatgacccgtgacaaagttacggttttgaatttgcaataa

SEQ ID NO: 50 - DNA, 4115 bp, synthetic construct
  source 1..4115: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatggcgggcacaggactattcgctgaaatattggatggtgaagtgtacaagtactatgcagatggagagtggaagaccagtagtagtggtaagtcggttgcaataatgaatcctgctacaagaaaaacccaatataaggttcaagcatgcacgcaagaagaagttaatgctgtgatggaattggccaagagcgctcaaaaatcatgggccaaaacaccattgtggaagcgtgctgagttactacataaagccgccgccatattaaaagacaataaggctcctatggcggagtctttagtcaaagagattgctaaacctgcgaaagacagtgttaccgaagttgtaaggtcaggagatctaatatcatattgcgctgaagagggagttcgtatattgggagaaggtaaattcttgctatctgattcatttccaggtaatgatagaacaaaatactgtctgacatcaaagatccctcttggagtagtgttagctatacccccattcaactatccagttaacctggctgtctcaaagatcgcaccagcgttaattgctggtaactcattggttttgaaacccccgacacagggtgcggtgtcttgtcttcacatggttcattgttttcatcttgcaggattcccaaagggtcttatttcttgcataaccggcaaaggcagtgaaataggagattttctaactatgcatcctgcagtaaactgtatttcatttacaggtggcgacactggaatatccataagtaaaaaagctggcatgatcccattacaaatggaattgggtgggaaggatgcatgtattgtattagatgatgctgatttggacttggtagcttcaaatatcattaaaggtggattttcctacagtggtcaaaggtgcacggcggttaaggttgttttggttatggagtcagtggctgatgaactagtcgaaaaggtcaaagcaaaagttgctaaattgacagtgggtccaccagaggagaactctgatattacggcggtagtctcggagagctccgctaattttattgaaggtttagtgatggatgcaaaggaaaaaggtgccacattctgtcaggaatacaagagagaaggaaacctaatctggcctttattgcttgacaatgttagacctgacatgagaatagcatgggaagaacccttcggacctgtcgtccctgttttgagaattaattcagttgaagaaggcattaaccactgtaacgcatctaacttcggtttgcaaggttgtgtgtttactaaagatattaataaggctattcttatttcggatgctatggagacaggcactgtacaaataaacagcgcaccagcaagaggtcctgatcacttccctttccaagggttgaaggattcaggcattggatcgcaaggggttaccaacagcataaatctaatgacgaaagtaaaaactacagttattaacctgcccactccaagctactctatgggctaacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 51 - DNA, 3904 bp, synthetic construct
  source 1..3904: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttagcccatagagtagcttggagtgggcaggttaataactgtagtttttactttcgtcattagatttatgctgttggtaaccccttgcgatccaatgcctgaatccttcaacccttggaaagggaagtgatcaggacctcttgctggtgcgctgtttatttgtacagtgcctgtctccatagcatccgaaataagaatagccttattaatatctttagtaaacacacaaccttgcaaaccgaagttagatgcgttacagtggttaatgccttcttcaactgaattaattctcaaaacagggacgacaggtccgaagggttcttcccatgctattctcatgtcaggtctaacattgtcaagcaataaaggccagattaggtttccttctctcttgtattcctgacagaatgtggcacctttttcctttgcatccatcactaaaccttcaataaaattagcggagctctccgagactaccgccgtaatatcagagttctcctctggtggacccactgtcaatttagcaacttttgctttgaccttttcgactagttcatcagccactgactccataaccaaaacaaccttaaccgccgtgcacctttgaccactgtaggaaaatccacctttaatgatatttgaagctaccaagtccaaatcagcatcatctaatacaatacatgcatccttcccacccaattccatttgtaatgggatcatgccagcttttttacttatggatattccagtgtcgccacctgtaaatgaaatacagtttactgcaggatgcatagttagaaaatctcctatttcactgcctttgccggttatgcaagaaataagaccctttgggaatcctgcaagatgaaaacaatgaaccatgtgaagacaagacaccgcaccctgtgtcgggggtttcaaaaccaatgagttaccagcaattaacgctggtgcgatctttgagacagccaggttaactggatagttgaatgggggtatagctaacactactccaagagggatctttgatgtcagacagtattttgttctatcattacctggaaatgaatcagatagcaagaatttaccttctcccaatatacgaactccctcttcagcgcaatatgatattagatctcctgaccttacaacttcggtaacactgtctttcgcaggtttagcaatctctttgactaaagactccgccataggagccttattgtcttttaatatggcggcggctttatgtagtaactcagcacgcttccacaatggtgttttggcccatgatttttgagcgctcttggccaattccatcacagcattaacttcttcttgcgtgcatgcttgaaccttatattgggtttttcttgtagcaggattcattattgcaaccgacttaccactactactggtcttccactctccatctgcatagtacttgtacacttcaccatccaatatttcagcgaatagtcctgtgcccgccatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 52 - protein, 496 aa, Arabidopsis thaliana
MAGTGLFAEILDGEVYKYYADGEWKTSSSGKSVAIMNPATRKTQYKVQACTQEEVNAVMELAKSAQKSWAKTPLWKRAELLHKAAAILKDNKAPMAESLVKEIAKPAKDSVTEVVRSGDLISYCAEEGVRILGEGKFLLSDSFPGNDRTKYCLTSKIPLGVVLAIPPFNYPVNLAVSKIAPALIAGNSLVLKPPTQGAVSCLHMVHCFHLAGFPKGLISCITGKGSEIGDFLTMHPAVNCISFTGGDTGISISKKAGMIPLQMELGGKDACIVLDDADLDLVASNIIKGGFSYSGQRCTAVKVVLVMESVADELVEKVKAKVAKLTVGPPEENSDITAVVSESSANFIEGLVMDAKEKGATFCQEYKREGNLIWPLLLDNVRPDMRIAWEEPFGPVVPVLRINSVEEGINHCNASNFGLQGCVFTKDINKAILISDAMETGTVQINSAPARGPDHFPFQGLKDSGIGSQGVTNSINLMTKVKTTVINLPTPSYSMG

SEQ ID NO: 53 - DNA, 1491 bp, synthetic construct
  source 1..1491: mol_type = other DNA
atggcgggcacaggactattcgctgaaatattggatggtgaagtgtacaagtactatgcagatggagagtggaagaccagtagtagtggtaagtcggttgcaataatgaatcctgctacaagaaaaacccaatataaggttcaagcatgcacgcaagaagaagttaatgctgtgatggaattggccaagagcgctcaaaaatcatgggccaaaacaccattgtggaagcgtgctgagttactacataaagccgccgccatattaaaagacaataaggctcctatggcggagtctttagtcaaagagattgctaaacctgcgaaagacagtgttaccgaagttgtaaggtcaggagatctaatatcatattgcgctgaagagggagttcgtatattgggagaaggtaaattcttgctatctgattcatttccaggtaatgatagaacaaaatactgtctgacatcaaagatccctcttggagtagtgttagctatacccccattcaactatccagttaacctggctgtctcaaagatcgcaccagcgttaattgctggtaactcattggttttgaaacccccgacacagggtgcggtgtcttgtcttcacatggttcattgttttcatcttgcaggattcccaaagggtcttatttcttgcataaccggcaaaggcagtgaaataggagattttctaactatgcatcctgcagtaaactgtatttcatttacaggtggcgacactggaatatccataagtaaaaaagctggcatgatcccattacaaatggaattgggtgggaaggatgcatgtattgtattagatgatgctgatttggacttggtagcttcaaatatcattaaaggtggattttcctacagtggtcaaaggtgcacggcggttaaggttgttttggttatggagtcagtggctgatgaactagtcgaaaaggtcaaagcaaaagttgctaaattgacagtgggtccaccagaggagaactctgatattacggcggtagtctcggagagctccgctaattttattgaaggtttagtgatggatgcaaaggaaaaaggtgccacattctgtcaggaatacaagagagaaggaaacctaatctggcctttattgcttgacaatgttagacctgacatgagaatagcatgggaagaacccttcggacctgtcgtccctgttttgagaattaattcagttgaagaaggcattaaccactgtaacgcatctaacttcggtttgcaaggttgtgtgtttactaaagatattaataaggctattcttatttcggatgctatggagacaggcactgtacaaataaacagcgcaccagcaagaggtcctgatcacttccctttccaagggttgaaggattcaggcattggatcgcaaggggttaccaacagcataaatctaatgacgaaagtaaaaactacagttattaacctgcccactccaagctactctatgggctaa

SEQ ID NO: 54 - DNA, 4145 bp, synthetic construct
  source 1..4145: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatggataaacttgtgcataataagatttttgacggtatttacaggcctggagaagacggtattcctgaatttaaggcctacgtggcagggcaatgggttttcggagagtcattcagtgaggtaaagtcacctattgacggtaggataatcgctagggtgtccagattaagtcgtgaccaagtagaagaggccctggctagggtctatgagaaaggaagagaagaaatacgtaactatccgggggaaaagagagtccaatcattcctgaaagcagctggtcttatgagggaagctttcgaggactttgtgaacgtccttattctagatgccggcaaaccgaaatcaaatgccgttggcgaaacaaaggcaactattgaaagactggagaaaaccacattcgaaagcagaagaatgttaggtgactacgtgcctggagactggtctgaagagactttagagtcagaaggaattgtaaaaagggaaccttacggactgattttggccatatctccatttaattacccgttattcatttccgcagctaaagttgttcctgctttactttccggtaataccgttttattgaaaccagcctctgcagacccattagcagctattatgtttgtcagagttttggaattggctggttttcctaaagaaagcttcgctcttttaacaatacccggttcgttcatggacgatgttgtgaaggatagaaggataagggctattacattcacgggatccacagaagtcggagaacatatcataagaacaggtgggattaaggcctaccacttagaactaggtggcaaagacccagctatcgtattggatgacgcgaatctagagttggcaagtgagaaaatcgttaagggtatggtttcattcagcggtcaacgttgtgatgctataagacttatattggcacaagaaaaaatttacgaggatctgaaagcacgtattcttgaaaagttgaagggtatggaacccaggaatccgttagaagatgagaacgccgttatgggccccttaattgatgaatcttctgccgaatacatagaggaagtctacaaggatgctgttgagaaaggtgctcgtcctcttacagaattcagaagaaaaggtaattatgtttggcctatattattagaagtttcgaaggaaaagatcaaagaccttagggcgtttaacgaagatgtctttggttccatggctttattgataaaagttaaagatgaagacgaggcgattgaaattgcaaagtcatctagatttggtttagacgcgtctgtgtttagtgaatgtgaacgtagagccagaaaggtagcaagaaagttagaggttggctccgtttttataaatgaataccccagacacggaattggctactacccttttggtggtatgaaagattcgggcgtcggtagagaaggtataggttactcggttgaacaattcacaactaccaagacgattgtgcacaattttaagggttatggagtatgggaatacctttaacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 55 - DNA, 3934 bp, synthetic construct
  source 1..3934: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttaaaggtattcccatactccataacccttaaaattgtgcacaatcgtcttggtagttgtgaattgttcaaccgagtaacctataccttctctaccgacgcccgaatctttcataccaccaaaagggtagtagccaattccgtgtctggggtattcatttataaaaacggagccaacctctaactttcttgctacctttctggctctacgttcacattcactaaacacagacgcgtctaaaccaaatctagatgactttgcaatttcaatcgcctcgtcttcatctttaacttttatcaataaagccatggaaccaaagacatcttcgttaaacgccctaaggtctttgatcttttccttcgaaacttctaataatataggccaaacataattaccttttcttctgaattctgtaagaggacgagcacctttctcaacagcatccttgtagacttcctctatgtattcggcagaagattcatcaattaaggggcccataacggcgttctcatcttctaacggattcctgggttccatacccttcaacttttcaagaatacgtgctttcagatcctcgtaaattttttcttgtgccaatataagtcttatagcatcacaacgttgaccgctgaatgaaaccatacccttaacgattttctcacttgccaactctagattcgcgtcatccaatacgatagctgggtctttgccacctagttctaagtggtaggccttaatcccacctgttcttatgatatgttctccgacttctgtggatcccgtgaatgtaatagcccttatccttctatccttcacaacatcgtccatgaacgaaccgggtattgttaaaagagcgaagctttctttaggaaaaccagccaattccaaaactctgacaaacataatagctgctaatgggtctgcagaggctggtttcaataaaacggtattaccggaaagtaaagcaggaacaactttagctgcggaaatgaataacgggtaattaaatggagatatggccaaaatcagtccgtaaggttccctttttacaattccttctgactctaaagtctcttcagaccagtctccaggcacgtagtcacctaacattcttctgctttcgaatgtggttttctccagtctttcaatagttgcctttgtttcgccaacggcatttgatttcggtttgccggcatctagaataaggacgttcacaaagtcctcgaaagcttccctcataagaccagctgctttcaggaatgattggactctcttttcccccggatagttacgtatttcttctcttcctttctcatagaccctagccagggcctcttctacttggtcacgacttaatctggacaccctagcgattatcctaccgtcaataggtgactttacctcactgaatgactctccgaaaacccattgccctgccacgtaggccttaaattcaggaataccgtcttctccaggcctgtaaataccgtcaaaaatcttattatgcacaagtttatccatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 56 - protein, 506 aa, Thermococcus sibiricus
MDKLVHNKIFDGIYRPGEDGIPEFKAYVAGQWVFGESFSEVKSPIDGRIIARVSRLSRDQVEEALARVYEKGREEIRNYPGEKRVQSFLKAAGLMREAFEDFVNVLILDAGKPKSNAVGETKATIERLEKTTFESRRMLGDYVPGDWSEETLESEGIVKREPYGLILAISPFNYPLFISAAKVVPALLSGNTVLLKPASADPLAAIMFVRVLELAGFPKESFALLTIPGSFMDDVVKDRRIRAITFTGSTEVGEHIIRTGGIKAYHLELGGKDPAIVLDDANLELASEKIVKGMVSFSGQRCDAIRLILAQEKIYEDLKARILEKLKGMEPRNPLEDENAVMGPLIDESSAEYIEEVYKDAVEKGARPLTEFRRKGNYVWPILLEVSKEKIKDLRAFNEDVFGSMALLIKVKDEDEAIEIAKSSRFGLDASVFSECERRARKVARKLEVGSVFINEYPRHGIGYYPFGGMKDSGVGREGIGYSVEQFTTTKTIVHNFKGYGVWEYL

SEQ ID NO: 57 - DNA, 1521 bp, synthetic construct
  source 1..1521: mol_type = other DNA
atggataaacttgtgcataataagatttttgacggtatttacaggcctggagaagacggtattcctgaatttaaggcctacgtggcagggcaatgggttttcggagagtcattcagtgaggtaaagtcacctattgacggtaggataatcgctagggtgtccagattaagtcgtgaccaagtagaagaggccctggctagggtctatgagaaaggaagagaagaaatacgtaactatccgggggaaaagagagtccaatcattcctgaaagcagctggtcttatgagggaagctttcgaggactttgtgaacgtccttattctagatgccggcaaaccgaaatcaaatgccgttggcgaaacaaaggcaactattgaaagactggagaaaaccacattcgaaagcagaagaatgttaggtgactacgtgcctggagactggtctgaagagactttagagtcagaaggaattgtaaaaagggaaccttacggactgattttggccatatctccatttaattacccgttattcatttccgcagctaaagttgttcctgctttactttccggtaataccgttttattgaaaccagcctctgcagacccattagcagctattatgtttgtcagagttttggaattggctggttttcctaaagaaagcttcgctcttttaacaatacccggttcgttcatggacgatgttgtgaaggatagaaggataagggctattacattcacgggatccacagaagtcggagaacatatcataagaacaggtgggattaaggcctaccacttagaactaggtggcaaagacccagctatcgtattggatgacgcgaatctagagttggcaagtgagaaaatcgttaagggtatggtttcattcagcggtcaacgttgtgatgctataagacttatattggcacaagaaaaaatttacgaggatctgaaagcacgtattcttgaaaagttgaagggtatggaacccaggaatccgttagaagatgagaacgccgttatgggccccttaattgatgaatcttctgccgaatacatagaggaagtctacaaggatgctgttgagaaaggtgctcgtcctcttacagaattcagaagaaaaggtaattatgtttggcctatattattagaagtttcgaaggaaaagatcaaagaccttagggcgtttaacgaagatgtctttggttccatggctttattgataaaagttaaagatgaagacgaggcgattgaaattgcaaagtcatctagatttggtttagacgcgtctgtgtttagtgaatgtgaacgtagagccagaaaggtagcaagaaagttagaggttggctccgtttttataaatgaataccccagacacggaattggctactacccttttggtggtatgaaagattcgggcgtcggtagagaaggtataggttactcggttgaacaattcacaactaccaagacgattgtgcacaattttaagggttatggagtatgggaatacctttaa

SEQ ID NO: 58 - DNA, 4079 bp, synthetic construct
  source 1..4079: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatggattctataaaaaatgaggtaaaaacatatccagcctaccttactggtgaatggcaagcttccaactcccaagaaacaatagcaattaagtctccttggaagcatgaggtgattgggaacgttcaagctgttacgcagtcagaagtcgatgaatccatcgaggctgctaaggctgctcaacactcttgggcaaaacttagtttgggtgatcgtggtcaatatttaaacagatgggctgatgaacttgaaaaaactaaagacgatatagcaacatctgtgatgaatgaagtaggcaagaattttaattccgcggaaggtgaagtgcaaagaactattgacttgataagatacactgttcaagaagctctgcatatgcacggggagagtgttagaggtgatggatttcctggaggatctaaggataaattaggtattatagaaagggtcccactgggggttgtcctagcgatatcgccatttaactatcctgttaacctggctgcatccaagatcgctccagctttaatggctggtaacgctgtcatatttaagccggccactcaaggctctatcagtggtatcaagatgatccaggcactggaccgtgctggattccccaaaggcatactatcgcttgtaactggtcacggctcagaaatcggtgattaccttacggaaaatccagatattaatatgatcagttttactggtagcactaaaaccggggagcacttaagccagaagagtatcatgacaccactagtcttagaacttggtggcaaagatcccgcaattgtctgcgaggatgctgatttaaatattacagcgaataacataatcaaaggagcttattcttattccggtcagagatgtaccgcggtaaagcgtgttttagtcgctgataaagtcgccgataaactggtggccagattaaagacacaagttgagaacttgtcggttgggagtcctgttaatgactccacgattgtacctttgattgacgataaggccgctaattttgttcagggcttgattgatgatgctttagccaagggtgccaccctggtgaccggtaacaaaagggacggaaatttgttgcaacctacattgctggataatgttactgaagacatggacattgcctgggttgagccattcggaccagtattgccaatcataagagtccactctgttgatcaagcagtggaaattgcgaacaagtctagattcggtttgcaggcaagcgttttcacacaagacatcgataaggcattggagatagcaggcgccgtagaagttggaaccgttcaggttaatggtagaccacagagaggtcctgataacttccctttcttaggtgttaaagccagtggcatgggaacacagggtgtgcataattcaatccttagtatgagtcgtgaaaaattaactgttattaacttgaagccataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 59 - DNA, 3868 bp, synthetic construct
  source 1..3868: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttatggcttcaagttaataacagttaatttttcacgactcatactaaggattgaattatgcacaccctgtgttcccatgccactggctttaacacctaagaaagggaagttatcaggacctctctgtggtctaccattaacctgaacggttccaacttctacggcgcctgctatctccaatgccttatcgatgtcttgtgtgaaaacgcttgcctgcaaaccgaatctagacttgttcgcaatttccactgcttgatcaacagagtggactcttatgattggcaatactggtccgaatggctcaacccaggcaatgtccatgtcttcagtaacattatccagcaatgtaggttgcaacaaatttccgtcccttttgttaccggtcaccagggtggcacccttggctaaagcatcatcaatcaagccctgaacaaaattagcggccttatcgtcaatcaaaggtacaatcgtggagtcattaacaggactcccaaccgacaagttctcaacttgtgtctttaatctggccaccagtttatcggcgactttatcagcgactaaaacacgctttaccgcggtacatctctgaccggaataagaataagctcctttgattatgttattcgctgtaatatttaaatcagcatcctcgcagacaattgcgggatctttgccaccaagttctaagactagtggtgtcatgatactcttctggcttaagtgctccccggttttagtgctaccagtaaaactgatcatattaatatctggattttccgtaaggtaatcaccgatttctgagccgtgaccagttacaagcgatagtatgcctttggggaatccagcacggtccagtgcctggatcatcttgataccactgatagagccttgagtggccggcttaaatatgacagcgttaccagccattaaagctggagcgatcttggatgcagccaggttaacaggatagttaaatggcgatatcgctaggacaacccccagtgggaccctttctataatacctaatttatccttagatcctccaggaaatccatcacctctaacactctccccgtgcatatgcagagcttcttgaacagtgtatcttatcaagtcaatagttctttgcacttcaccttccgcggaattaaaattcttgcctacttcattcatcacagatgttgctatatcgtctttagttttttcaagttcatcagcccatctgtttaaatattgaccacgatcacccaaactaagttttgcccaagagtgttgagcagccttagcagcctcgatggattcatcgacttctgactgcgtaacagcttgaacgttcccaatcacctcatgcttccaaggagacttaattgctattgtttcttgggagttggaagcttgccattcaccagtaaggtaggctggatatgtttttacctcattttttatagaatccatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 60 - protein, 484 aa, Lactobacillus kisonensis
MDSIKNEVKTYPAYLTGEWQASNSQETIAIKSPWKHEVIGNVQAVTQSEVDESIEAAKAAQHSWAKLSLGDRGQYLNRWADELEKTKDDIATSVMNEVGKNFNSAEGEVQRTIDLIRYTVQEALHMHGESVRGDGFPGGSKDKLGIIERVPLGVVLAISPFNYPVNLAASKIAPALMAGNAVIFKPATQGSISGIKMIQALDRAGFPKGILSLVTGHGSEIGDYLTENPDINMISFTGSTKTGEHLSQKSIMTPLVLELGGKDPAIVCEDADLNITANNIIKGAYSYSGQRCTAVKRVLVADKVADKLVARLKTQVENLSVGSPVNDSTIVPLIDDKAANFVQGLIDDALAKGATLVTGNKRDGNLLQPTLLDNVTEDMDIAWVEPFGPVLPIIRVHSVDQAVEIANKSRFGLQASVFTQDIDKALEIAGAVEVGTVQVNGRPQRGPDNFPFLGVKASGMGTQGVHNSILSMSREKLTVINLKP

SEQ ID NO: 61 - DNA, 1455 bp, synthetic construct
  source 1..1455: mol_type = other DNA
atggattctataaaaaatgaggtaaaaacatatccagcctaccttactggtgaatggcaagcttccaactcccaagaaacaatagcaattaagtctccttggaagcatgaggtgattgggaacgttcaagctgttacgcagtcagaagtcgatgaatccatcgaggctgctaaggctgctcaacactcttgggcaaaacttagtttgggtgatcgtggtcaatatttaaacagatgggctgatgaacttgaaaaaactaaagacgatatagcaacatctgtgatgaatgaagtaggcaagaattttaattccgcggaaggtgaagtgcaaagaactattgacttgataagatacactgttcaagaagctctgcatatgcacggggagagtgttagaggtgatggatttcctggaggatctaaggataaattaggtattatagaaagggtcccactgggggttgtcctagcgatatcgccatttaactatcctgttaacctggctgcatccaagatcgctccagctttaatggctggtaacgctgtcatatttaagccggccactcaaggctctatcagtggtatcaagatgatccaggcactggaccgtgctggattccccaaaggcatactatcgcttgtaactggtcacggctcagaaatcggtgattaccttacggaaaatccagatattaatatgatcagttttactggtagcactaaaaccggggagcacttaagccagaagagtatcatgacaccactagtcttagaacttggtggcaaagatcccgcaattgtctgcgaggatgctgatttaaatattacagcgaataacataatcaaaggagcttattcttattccggtcagagatgtaccgcggtaaagcgtgttttagtcgctgataaagtcgccgataaactggtggccagattaaagacacaagttgagaacttgtcggttgggagtcctgttaatgactccacgattgtacctttgattgacgataaggccgctaattttgttcagggcttgattgatgatgctttagccaagggtgccaccctggtgaccggtaacaaaagggacggaaatttgttgcaacctacattgctggataatgttactgaagacatggacattgcctgggttgagccattcggaccagtattgccaatcataagagtccactctgttgatcaagcagtggaaattgcgaacaagtctagattcggtttgcaggcaagcgttttcacacaagacatcgataaggcattggagatagcaggcgccgtagaagttggaaccgttcaggttaatggtagaccacagagaggtcctgataacttccctttcttaggtgttaaagccagtggcatgggaacacagggtgtgcataattcaatccttagtatgagtcgtgaaaaattaactgttattaacttgaagccataa

SEQ ID NO: 62 - DNA, 4253 bp, synthetic construct
  source 1..4253: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgtcatccgaaaataccgcaagttttcaccatatcttcaaatcagagaacgaaatccctgaagaatataaagttccagttatacaccagcgtacgtatctgttgaacggggaattagtggaatggaacggggacgtgactgaaatctattcaccagtttgtattccaacagaaaatggcttggaaaggaaattattgggaagtattccaaatataggacctaatgaagctatggaagttcttgaggcttgcgtaaaagcatacgataatgggttgggagaatggccgactatgtcagtggaagggagaattaagtgtatgcaaaagttcgtttatctgatgatacaacaaagagatttaattatcaaactattgatgtgggagattggtaagactttggctgattctacaaaagagtttgataggacggtcgattatattaatcaaacaattgacgccttgaaggatctagatagagaatcttctcgttttcaacaggccgaaggtaccatcgcccaaattaggagagcgcctttgggagtcgtgctgagtatgggtcccttcaattatccgttgaatgaaatcttcacaactttaatccctgcattaataatgggtaatacaattttattcaagttacctaagcatggtgttttggcccattacccccttctagaagcatttaaagaagcttttccaaagggaaccgtaaacacgttgtacggtaagggcagtgaaattataactccaattatggagtccgggaaagtaaacgtgcttgcctttattggttccagcaaggtagctaatggactaaaaaaactacatccaaaggtgaataggctgagagcaattctgagcctagatgcaaagaatgcagcaattgtcacaaagaacgctaatttggacgtggcagtttcagaatgtatattaggagctcttagtttcaatggccagcgttgtactgcattaaaacttatctttgtccagaaggaggttgcactggaatttaccgaaaagttatcagcggctgtgtcggcattgaagcctggattaccttgggaaaaagatgttaaagtaacacctctaccagaagttaataagcctccatatttgaaagaatgcatagatgatgctttgcagaagggagccgcagttttaaataaagatggtgggtataccgatgaatcttttgtatttcccgccgttgtataccccgttaattctgatatgaagctgtatcacgaagaacaatttggtccagtaataccggtagtgccctttgaagatatcgaggagcccattgagtatcaagtcaatgcctctcatggtatgcaagtatcaattttttcagaagacccacaggaggtggcaagattgattgatccctttgtgaatttagtgtctagagtcaacatcaactgccaagcacaaagagggccggatgtgtttccattcactggaaggaaagacagtgcggaaggtaccttgtctgtgttcgacgctttgagaagtttctctatccgtagtcttgttgcagcaaaactgactgattctaacaaagaacttttaaatacaatcgtaagagaacacgacagtaactttctaagcactgattacatcttctaacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 63 - DNA, 4042 bp, synthetic construct
  source 1..4042: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttagaagatgtaatcagtgcttagaaagttactgtcgtgttctcttacgattgtatttaaaagttctttgttagaatcagtcagttttgctgcaacaagactacggatagagaaacttctcaaagcgtcgaacacagacaaggtaccttccgcactgtctttccttccagtgaatggaaacacatccggccctctttgtgcttggcagttgatgttgactctagacactaaattcacaaagggatcaatcaatcttgccacctcctgtgggtcttctgaaaaaattgatacttgcataccatgagaggcattgacttgatactcaatgggctcctcgatatcttcaaagggcactaccggtattactggaccaaattgttcttcgtgatacagcttcatatcagaattaacggggtatacaacggcgggaaatacaaaagattcatcggtatacccaccatctttatttaaaactgcggctcccttctgcaaagcatcatctatgcattctttcaaatatggaggcttattaacttctggtagaggtgttactttaacatctttttcccaaggtaatccaggcttcaatgccgacacagccgctgataacttttcggtaaattccagtgcaacctccttctggacaaagataagttttaatgcagtacaacgctggccattgaaactaagagctcctaatatacattctgaaactgccacgtccaaattagcgttctttgtgacaattgctgcattctttgcatctaggctcagaattgctctcagcctattcacctttggatgtagtttttttagtccattagctaccttgctggaaccaataaaggcaagcacgtttactttcccggactccataattggagttataatttcactgcccttaccgtacaacgtgtttacggttccctttggaaaagcttctttaaatgcttctagaagggggtaatgggccaaaacaccatgcttaggtaacttgaataaaattgtattacccattattaatgcagggattaaagttgtgaagatttcattcaacggataattgaagggacccatactcagcacgactcccaaaggcgctctcctaatttgggcgatggtaccttcggcctgttgaaaacgagaagattctctatctagatccttcaaggcgtcaattgtttgattaatataatcgaccgtcctatcaaactcttttgtagaatcagccaaagtcttaccaatctcccacatcaatagtttgataattaaatctctttgttgtatcatcagataaacgaacttttgcatacacttaattctcccttccactgacatagtcggccattctcccaacccattatcgtatgcttttacgcaagcctcaagaacttccatagcttcattaggtcctatatttggaatacttcccaataatttcctttccaagccattttctgttggaatacaaactggtgaatagatttcagtcacgtccccgttccattccactaattccccgttcaacagatacgtacgctggtgtataactggaactttatattcttcagggatttcgttctctgatttgaagatatggtgaaaacttgcggtattttcggatgacatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 64 - protein, 542 aa, Chryseobacterium gleum
MSSENTASFHHIFKSENEIPEEYKVPVIHQRTYLLNGELVEWNGDVTEIYSPVCIPTENGLERKLLGSIPNIGPNEAMEVLEACVKAYDNGLGEWPTMSVEGRIKCMQKFVYLMIQQRDLIIKLLMWEIGKTLADSTKEFDRTVDYINQTIDALKDLDRESSRFQQAEGTIAQIRRAPLGVVLSMGPFNYPLNEIFTTLIPALIMGNTILFKLPKHGVLAHYPLLEAFKEAFPKGTVNTLYGKGSEIITPIMESGKVNVLAFIGSSKVANGLKKLHPKVNRLRAILSLDAKNAAIVTKNANLDVAVSECILGALSFNGQRCTALKLIFVQKEVALEFTEKLSAAVSALKPGLPWEKDVKVTPLPEVNKPPYLKECIDDALQKGAAVLNKDGGYTDESFVFPAVVYPVNSDMKLYHEEQFGPVIPVVPFEDIEEPIEYQVNASHGMQVSIFSEDPQEVARLIDPFVNLVSRVNINCQAQRGPDVFPFTGRKDSAEGTLSVFDALRSFSIRSLVAAKLTDSNKELLNTIVREHDSNFLSTDYIF

SEQ ID NO: 65 - DNA, 1629 bp, synthetic construct
  source 1..1629: mol_type = other DNA
atgtcatccgaaaataccgcaagttttcaccatatcttcaaatcagagaacgaaatccctgaagaatataaagttccagttatacaccagcgtacgtatctgttgaacggggaattagtggaatggaacggggacgtgactgaaatctattcaccagtttgtattccaacagaaaatggcttggaaaggaaattattgggaagtattccaaatataggacctaatgaagctatggaagttcttgaggcttgcgtaaaagcatacgataatgggttgggagaatggccgactatgtcagtggaagggagaattaagtgtatgcaaaagttcgtttatctgatgatacaacaaagagatttaattatcaaactattgatgtgggagattggtaagactttggctgattctacaaaagagtttgataggacggtcgattatattaatcaaacaattgacgccttgaaggatctagatagagaatcttctcgttttcaacaggccgaaggtaccatcgcccaaattaggagagcgcctttgggagtcgtgctgagtatgggtcccttcaattatccgttgaatgaaatcttcacaactttaatccctgcattaataatgggtaatacaattttattcaagttacctaagcatggtgttttggcccattacccccttctagaagcatttaaagaagcttttccaaagggaaccgtaaacacgttgtacggtaagggcagtgaaattataactccaattatggagtccgggaaagtaaacgtgcttgcctttattggttccagcaaggtagctaatggactaaaaaaactacatccaaaggtgaataggctgagagcaattctgagcctagatgcaaagaatgcagcaattgtcacaaagaacgctaatttggacgtggcagtttcagaatgtatattaggagctcttagtttcaatggccagcgttgtactgcattaaaacttatctttgtccagaaggaggttgcactggaatttaccgaaaagttatcagcggctgtgtcggcattgaagcctggattaccttgggaaaaagatgttaaagtaacacctctaccagaagttaataagcctccatatttgaaagaatgcatagatgatgctttgcagaagggagccgcagttttaaataaagatggtgggtataccgatgaatcttttgtatttcccgccgttgtataccccgttaattctgatatgaagctgtatcacgaagaacaatttggtccagtaataccggtagtgccctttgaagatatcgaggagcccattgagtatcaagtcaatgcctctcatggtatgcaagtatcaattttttcagaagacccacaggaggtggcaagattgattgatccctttgtgaatttagtgtctagagtcaacatcaactgccaagcacaaagagggccggatgtgtttccattcactggaaggaaagacagtgcggaaggtaccttgtctgtgttcgacgctttgagaagtttctctatccgtagtcttgttgcagcaaaactgactgattctaacaaagaacttttaaatacaatcgtaagagaacacgacagtaactttctaagcactgattacatcttctaa

SEQ ID NO: 66 - DNA, 4052 bp, synthetic construct
  source 1..4052: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgactaaacaatacaagaactatgtaaatggtgaatggaagttatctgaaaacgaaatcaaaatctacgagccagcttccggagcggagttaggttcagttccggcgatgagcactgaggaagtcgactatgtttacgcttctgcaaagaaagcacaaccagcctggcgttccttatcatatatcgagagagcagcgtacttgcacaaggtggcagatatactgatgagggataaggaaaaaattggtgctgttctgtccaaggaagttgccaagggatataaatctgcagtttcggaggttgttagaactgcagaaattattaattacgcagccgaagagggtttaagaatggaaggagaggtactagaaggaggttcgtttgaggctgcgtctaagaagaaaattgctgttgttcgtagagaacccgtcggcttagtgttggccatttcaccttttaattaccctgttaaccttgctgggtccaagattgcacccgctttgatcgctggaaacgttattgcttttaaacctccaactcaaggctcaatctcggggttgcttcttgcggaggcattcgctgaggctgggttgccagctggagtctttaataccattactgggcgtggttctgaaataggagattatatcgtcgaacatcaagctgtaaatttcataaactttacagggtcgactggtataggcgaaagaatcggtaaaatggccggaatgaggccaattatgttagaattaggaggaaaagactcagcaatcgtgttggaagatgctgatttagagttaaccgccaagaatatcattgcaggtgcttttggctatagcgggcaaagatgcacagcagttaaaagagtcttggttatggaaagtgttgcggatgaattagtggagaagatcagagaaaaggtactggcactgactattggtaaccctgaagatgatgctgacattaccccactaatagatactaagtcagcagattacgttgaaggtctgataaatgacgcaaatgacaagggtgcggctgcactaacagagattaaaagagaggggaacctgatttgcccaatactttttgacaaagtcactacagatatgcgtctggcatgggaggaaccattcggtccagtactacctattattagagttactagtgtagaagaagctattgaaatctccaataagtccgaatacggattacaagcatccatctttactaatgattttcccagagccttcgggatcgcagaacaattagaagttggtactgtgcatattaataacaaaacccagagaggtaccgacaacttcccgttcttaggagccaagaagtcaggagctggaattcaaggagttaaatacagtatagaggctatgacaaccgtgaagtcagtagtgtttgacatcaaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 67 - DNA, 3841 bp, synthetic construct
  source 1..3841: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttatttgatgtcaaacactactgacttcacggttgtcatagcctctatactgtatttaactccttgaattccagctcctgacttcttggctcctaagaacgggaagttgtcggtacctctctgggttttgttattaatatgcacagtaccaacttctaattgttctgcgatcccgaaggctctgggaaaatcattagtaaagatggatgcttgtaatccgtattcggacttattggagatttcaatagcttcttctacactagtaactctaataataggtagtactggaccgaatggttcctcccatgccagacgcatatctgtagtgactttgtcaaaaagtattgggcaaatcaggttcccctctcttttaatctctgttagtgcagccgcacccttgtcatttgcgtcatttatcagaccttcaacgtaatctgctgacttagtatctattagtggggtaatgtcagcatcatcttcagggttaccaatagtcagtgccagtaccttttctctgatcttctccactaattcatccgcaacactttccataaccaagactcttttaactgctgtgcatctttgcccgctatagccaaaagcacctgcaatgatattcttggcggttaactctaaatcagcatcttccaacacgattgctgagtcttttcctcctaattctaacataattggcctcattccggccattttaccgattctttcgcctataccagtcgaccctgtaaagtttatgaaatttacagcttgatgttcgacgatataatctcctatttcagaaccacgcccagtaatggtattaaagactccagctggcaacccagcctcagcgaatgcctccgcaagaagcaaccccgagattgagccttgagttggaggtttaaaagcaataacgtttccagcgatcaaagcgggtgcaatcttggacccagcaaggttaacagggtaattaaaaggtgaaatggccaacactaagccgacgggttctctacgaacaacagcaattttcttcttagacgcagcctcaaacgaacctccttctagtacctctccttccattcttaaaccctcttcggctgcgtaattaataatttctgcagttctaacaacctccgaaactgcagatttatatcccttggcaacttccttggacagaacagcaccaattttttccttatccctcatcagtatatctgccaccttgtgcaagtacgctgctctctcgatatatgataaggaacgccaggctggttgtgctttctttgcagaagcgtaaacatagtcgacttcctcagtgctcatcgccggaactgaacctaactccgctccggaagctggctcgtagattttgatttcgttttcagataacttccattcaccatttacatagttcttgtattgtttagtcatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 68 - protein, 475 aa, Streptococcus mutans
MTKQYKNYVNGEWKLSENEIKIYEPASGAELGSVPAMSTEEVDYVYASAKKAQPAWRSLSYIERAAYLHKVADILMRDKEKIGAVLSKEVAKGYKSAVSEVVRTAEIINYAAEEGLRMEGEVLEGGSFEAASKKKIAVVRREPVGLVLAISPFNYPVNLAGSKIAPALIAGNVIAFKPPTQGSISGLLLAEAFAEAGLPAGVFNTITGRGSEIGDYIVEHQAVNFINFTGSTGIGERIGKMAGMRPIMLELGGKDSAIVLEDADLELTAKNIIAGAFGYSGQRCTAVKRVLVMESVADELVEKIREKVLALTIGNPEDDADITPLIDTKSADYVEGLINDANDKGAAALTEIKREGNLICPILFDKVTTDMRLAWEEPFGPVLPIIRVTSVEEAIEISNKSEYGLQASIFTNDFPRAFGIAEQLEVGTVHINNKTQRGTDNFPFLGAKKSGAGIQGVKYSIEAMTTVKSVVFDIK

SEQ ID NO: 69 - DNA, 1428 bp, synthetic construct
  source 1..1428: mol_type = other DNA
atgactaaacaatacaagaactatgtaaatggtgaatggaagttatctgaaaacgaaatcaaaatctacgagccagcttccggagcggagttaggttcagttccggcgatgagcactgaggaagtcgactatgtttacgcttctgcaaagaaagcacaaccagcctggcgttccttatcatatatcgagagagcagcgtacttgcacaaggtggcagatatactgatgagggataaggaaaaaattggtgctgttctgtccaaggaagttgccaagggatataaatctgcagtttcggaggttgttagaactgcagaaattattaattacgcagccgaagagggtttaagaatggaaggagaggtactagaaggaggttcgtttgaggctgcgtctaagaagaaaattgctgttgttcgtagagaacccgtcggcttagtgttggccatttcaccttttaattaccctgttaaccttgctgggtccaagattgcacccgctttgatcgctggaaacgttattgcttttaaacctccaactcaaggctcaatctcggggttgcttcttgcggaggcattcgctgaggctgggttgccagctggagtctttaataccattactgggcgtggttctgaaataggagattatatcgtcgaacatcaagctgtaaatttcataaactttacagggtcgactggtataggcgaaagaatcggtaaaatggccggaatgaggccaattatgttagaattaggaggaaaagactcagcaatcgtgttggaagatgctgatttagagttaaccgccaagaatatcattgcaggtgcttttggctatagcgggcaaagatgcacagcagttaaaagagtcttggttatggaaagtgttgcggatgaattagtggagaagatcagagaaaaggtactggcactgactattggtaaccctgaagatgatgctgacattaccccactaatagatactaagtcagcagattacgttgaaggtctgataaatgacgcaaatgacaagggtgcggctgcactaacagagattaaaagagaggggaacctgatttgcccaatactttttgacaaagtcactacagatatgcgtctggcatgggaggaaccattcggtccagtactacctattattagagttactagtgtagaagaagctattgaaatctccaataagtccgaatacggattacaagcatccatctttactaatgattttcccagagccttcgggatcgcagaacaattagaagttggtactgtgcatattaataacaaaacccagagaggtaccgacaacttcccgttcttaggagccaagaagtcaggagctggaattcaaggagttaaatacagtatagaggctatgacaaccgtgaagtcagtagtgtttgacatcaaataa

SEQ ID NO: 70 - DNA, 4064 bp, synthetic construct
  source 1..4064: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgaaaacagctatcacctacacaagcctgattaatggtagatggattgagtcagaatctaatgaaactatcgaaatcagttcgccatacaaggaggaattagtaggtagagtgcaagcaatgaccaaaaatgaggtcgatgaagctatcagatctgctaaagttgcgcaaagaaactgggccagggtcccagctcataagagagcaagcttactacatgaatgggcttctgaattagaaaggagagccgagcaaatagggagagtgataatgcttgaagtaggtaaaggtttggcggacggagtgaaagaagttattagaactgccgagataataaggtatacagctgaagaagggttgcgttttgccggaggaatgatgcaaggtgacagttttccgggtggctccgctaacaaactagctgttataaaaaaagttccgttgggtgttgtgttagcaatttcaccatttaactatcccgttaacttggctgccgcaaagttagcccctgccttgatgactggtaacgcagttgtctttaaaccggcaacgcaaggcagtataagtggtaccttgatgattgaagcactggttgaagctggcttacctaaaggtttagtgaatttggtcacaggtagaggaagtgtaataggggattacttgacaactcatcctggtatagatatgataacattcacaggtggaacgtccacaggtcaacaccttagtcgtaggagcgcaatggttcctttggtgttagaattaggtggtaaagacccagctttggttttagaggatgctgatatggaattggctgcagatcaaatagtgtcaggagctttttcttatagcggtcaaagatgtacggccattaagcgtgtctttgtcttggagaatcaagctgatgagttgattgcacttgtgaaggcgagagttgaaaaattatcagtgggtagcccagaagaaaattcagttatcgtccctttaattgataagaaatcggcggatttcgttgagggtctaataactgatgcacgtgataaaggagcaaaggttattcacggtggcacccgtgaaggtaatttgattgctcctactttattggacgacgttaccacagaaatgagagtcgcatgggaagaaccttttggtccggtccttcctattataagagtgaagtctgtggatgaaatggtagatctagccaacgaatccgaatatgggttacaagcttcggtttttaccagagatatcgacgcagcttttaggatagctgatgctctagaaactggaagcgttcaaattaatggtagaacagaaagaggcccagatcattttcctttcattggtgtcaaatctagtgggttgggtgtccaaggcgtaggtagatcacttgaatcaatgacaagagataaactgaccgtattaaacttgcaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 71 - DNA, 3853 bp, synthetic construct
  source 1..3853: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttattgcaagtttaatacggtcagtttatctcttgtcattgattcaagtgatctacctacgccttggacacccaacccactagatttgacaccaatgaaaggaaaatgatctgggcctctttctgttctaccattaatttgaacgcttccagtttctagagcatcagctatcctaaaagctgcgtcgatatctctggtaaaaaccgaagcttgtaacccatattcggattcgttggctagatctaccatttcatccacagacttcactcttataataggaaggaccggaccaaaaggttcttcccatgcgactctcatttctgtggtaacgtcgtccaataaagtaggagcaatcaaattaccttcacgggtgccaccgtgaataacctttgctcctttatcacgtgcatcagttattagaccctcaacgaaatccgccgatttcttatcaattaaagggacgataactgaattttcttctgggctacccactgataatttttcaactctcgccttcacaagtgcaatcaactcatcagcttgattctccaagacaaagacacgcttaatggccgtacatctttgaccgctataagaaaaagctcctgacactatttgatctgcagccaattccatatcagcatcctctaaaaccaaagctgggtctttaccacctaattctaacaccaaaggaaccattgcgctcctacgactaaggtgttgacctgtggacgttccacctgtgaatgttatcatatctataccaggatgagttgtcaagtaatcccctattacacttcctctacctgtgaccaaattcactaaacctttaggtaagccagcttcaaccagtgcttcaatcatcaaggtaccacttatactgccttgcgttgccggtttaaagacaactgcgttaccagtcatcaaggcaggggctaactttgcggcagccaagttaacgggatagttaaatggtgaaattgctaacacaacacccaacggaactttttttataacagctagtttgttagcggagccacccggaaaactgtcaccttgcatcattcctccggcaaaacgcaacccttcttcagctgtataccttattatctcggcagttctaataacttctttcactccgtccgccaaacctttacctacttcaagcattatcactctccctatttgctcggctctcctttctaattcagaagcccattcatgtagtaagcttgctctcttatgagctgggaccctggcccagtttctttgcgcaactttagcagatctgatagcttcatcgacctcatttttggtcattgcttgcactctacctactaattcctccttgtatggcgaactgatttcgatagtttcattagattctgactcaatccatctaccattaatcaggcttgtgtaggtgatagctgttttcatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 72 - protein, 479 aa, Exiguobacterium flavidum
MKTAITYTSLINGRWIESESNETIEISSPYKEELVGRVQAMTKNEVDEAIRSAKVAQRNWARVPAHKRASLLHEWASELERRAEQIGRVIMLEVGKGLADGVKEVIRTAEIIRYTAEEGLRFAGGMMQGDSFPGGSANKLAVIKKVPLGVVLAISPFNYPVNLAAAKLAPALMTGNAVVFKPATQGSISGTLMIEALVEAGLPKGLVNLVTGRGSVIGDYLTTHPGIDMITFTGGTSTGQHLSRRSAMVPLVLELGGKDPALVLEDADMELAADQIVSGAFSYSGQRCTAIKRVFVLENQADELIALVKARVEKLSVGSPEENSVIVPLIDKKSADFVEGLITDARDKGAKVIHGGTREGNLIAPTLLDDVTTEMRVAWEEPFGPVLPIIRVKSVDEMVDLANESEYGLQASVFTRDIDAAFRIADALETGSVQINGRTERGPDHFPFIGVKSSGLGVQGVGRSLESMTRDKLTVLNLQ

SEQ ID NO: 73 - DNA, 1440 bp, synthetic construct
  source 1..1440: mol_type = other DNA
atgaaaacagctatcacctacacaagcctgattaatggtagatggattgagtcagaatctaatgaaactatcgaaatcagttcgccatacaaggaggaattagtaggtagagtgcaagcaatgaccaaaaatgaggtcgatgaagctatcagatctgctaaagttgcgcaaagaaactgggccagggtcccagctcataagagagcaagcttactacatgaatgggcttctgaattagaaaggagagccgagcaaatagggagagtgataatgcttgaagtaggtaaaggtttggcggacggagtgaaagaagttattagaactgccgagataataaggtatacagctgaagaagggttgcgttttgccggaggaatgatgcaaggtgacagttttccgggtggctccgctaacaaactagctgttataaaaaaagttccgttgggtgttgtgttagcaatttcaccatttaactatcccgttaacttggctgccgcaaagttagcccctgccttgatgactggtaacgcagttgtctttaaaccggcaacgcaaggcagtataagtggtaccttgatgattgaagcactggttgaagctggcttacctaaaggtttagtgaatttggtcacaggtagaggaagtgtaataggggattacttgacaactcatcctggtatagatatgataacattcacaggtggaacgtccacaggtcaacaccttagtcgtaggagcgcaatggttcctttggtgttagaattaggtggtaaagacccagctttggttttagaggatgctgatatggaattggctgcagatcaaatagtgtcaggagctttttcttatagcggtcaaagatgtacggccattaagcgtgtctttgtcttggagaatcaagctgatgagttgattgcacttgtgaaggcgagagttgaaaaattatcagtgggtagcccagaagaaaattcagttatcgtccctttaattgataagaaatcggcggatttcgttgagggtctaataactgatgcacgtgataaaggagcaaaggttattcacggtggcacccgtgaaggtaatttgattgctcctactttattggacgacgttaccacagaaatgagagtcgcatgggaagaaccttttggtccggtccttcctattataagagtgaagtctgtggatgaaatggtagatctagccaacgaatccgaatatgggttacaagcttcggtttttaccagagatatcgacgcagcttttaggatagctgatgctctagaaactggaagcgttcaaattaatggtagaacagaaagaggcccagatcattttcctttcattggtgtcaaatctagtgggttgggtgtccaaggcgtaggtagatcacttgaatcaatgacaagagataaactgaccgtattaaacttgcaataa

SEQ ID NO: 74 - DNA, 4205 bp, synthetic construct
  source 1..4205: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgtctttgattccggaggaatttcaaataaaaaatttgataaaccaagatacttaccttgtaaatggggaacttaaaccctggacaggccaaacgacccctgtcttctctacgatttcctccacggaaaagtatactcccacattattaggcagtattcctttcatggaggaaaaagaagcagcggaagtagtagaagcagcgaacacagcatataacaaggggcagggactttggccaactatgaaagttgtagatcgtataaagtgcatggaaaattttgttaaccaaatgaaagagactagggaagaagttgtaaaattgctgatgtgggaaatagggaaaaatcttggtgactcccaaaaagagtttgatagaacggttgaatatatacaagatacaatcgatagctataaagagcttaacggaagatccagccactttgaaaaagttcagggcgttaacgccatgattaggaggggtccattaggggtcgttttatgcctgggtccatataattacccactaaatgagacgttttctttgttaataccggctttaattatgggcaacacagttatcttcaaaccggctaaacatggtgtattatgtattagccctcttcttgaggctttcagatcgagctttcccaagggagttattaacattgtgtatggtcgtgggagagaggtagcatctccgatcatgaagagcgggaaaatcgacgttttagcattaattggcaactcaaaatcagcaattgctttacaagatcaacatccaaacaagaatagattgagattaattttaggcctggaagccaaaaatccagctattattcttccagacgcagatcttgatttagcgatccaagagtgtatcgctggcacattatcgtttaacggacaaagatgtaccgctttgaaagtcctttatgtgcacgaatccattgctgaggagtttaacaagaggttcgccgaaaaggtggacggtttagcttttggcaatccatgggagaagggtgtttcactgacacctttacctgaaactgacaagccgaagtatatacaaggtctgattgacgatgccacccacaagggtgcaaagatcctaaatgaaaaaggtggtaagcatacagataattacattttcccagctgtactgtatcctgtgaataaggaaatgagagtatatcatgaggagcagtttggcccagttgtgcccataatttctttcaaggacattaatgagccacttgaagacatggctgaatcgaattatggtcaacaggtttcattgtttggtaaagacataaaaactttggcacctttaatcgacgctctagtaaatctagtgtgcagggtcaaccttaactcaagttgtcaaagaggacctgacgcatttccatttacaggtcgtaaggattccgctgtagggacattgtctataccggatgccttgaggtcatttagcattagaacttttgttgctagtaaggacatcgcatacaataatgaaatattgcaggaattattaaactctcgtgagagtaatttcattaacacagattatatactttaacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 75 - DNA, 3994 bp, synthetic construct
  source 1..3994: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttaaagtatataatctgtgttaatgaaattactctcacgagagtttaataattcctgcaatatttcattattgtatgcgatgtccttactagcaacaaaagttctaatgctaaatgacctcaaggcatccggtatagacaatgtccctacagcggaatccttacgacctgtaaatggaaatgcgtcaggtcctctttgacaacttgagttaaggttgaccctgcacactagatttactagagcgtcgattaaaggtgccaaagtttttatgtctttaccaaacaatgaaacctgttgaccataattcgattcagccatgtcttcaagtggctcattaatgtccttgaaagaaattatgggcacaactgggccaaactgctcctcatgatatactctcatttccttattcacaggatacagtacagctgggaaaatgtaattatctgtatgcttaccacctttttcatttaggatctttgcacccttgtgggtggcatcgtcaatcagaccttgtatatacttcggcttgtcagtttcaggtaaaggtgtcagtgaaacacccttctcccatggattgccaaaagctaaaccgtccaccttttcggcgaacctcttgttaaactcctcagcaatggattcgtgcacataaaggactttcaaagcggtacatctttgtccgttaaacgataatgtgccagcgatacactcttggatcgctaaatcaagatctgcgtctggaagaataatagctggatttttggcttccaggcctaaaattaatctcaatctattcttgtttggatgttgatcttgtaaagcaattgctgattttgagttgccaattaatgctaaaacgtcgattttcccgctcttcatgatcggagatgctacctctctcccacgaccatacacaatgttaataactcccttgggaaagctcgatctgaaagcctcaagaagagggctaatacataatacaccatgtttagccggtttgaagataactgtgttgcccataattaaagccggtattaacaaagaaaacgtctcatttagtgggtaattatatggacccaggcataaaacgacccctaatggacccctcctaatcatggcgttaacgccctgaactttttcaaagtggctggatcttccgttaagctctttatagctatcgattgtatcttgtatatattcaaccgttctatcaaactctttttgggagtcaccaagatttttccctatttcccacatcagcaattttacaacttcttccctagtctctttcatttggttaacaaaattttccatgcactttatacgatctacaactttcatagttggccaaagtccctgccccttgttatatgctgtgttcgctgcttctactacttccgctgcttctttttcctccatgaaaggaatactgcctaataatgtgggagtatacttttccgtggaggaaatcgtagagaagacaggggtcgtttggcctgtccagggtttaagttccccatttacaaggtaagtatcttggtttatcaaattttttatttgaaattcctccggaatcaaagacatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 76 - protein, 526 aa, Flavobacterium collinsii
MSLIPEEFQIKNLINQDTYLVNGELKPWTGQTTPVFSTISSTEKYTPTLLGSIPFMEEKEAAEVVEAANTAYNKGQGLWPTMKVVDRIKCMENFVNQMKETREEVVKLLMWEIGKNLGDSQKEFDRTVEYIQDTIDSYKELNGRSSHFEKVQGVNAMIRRGPLGVVLCLGPYNYPLNETFSLLIPALIMGNTVIFKPAKHGVLCISPLLEAFRSSFPKGVINIVYGRGREVASPIMKSGKIDVLALIGNSKSAIALQDQHPNKNRLRLILGLEAKNPAIILPDADLDLAIQECIAGTLSFNGQRCTALKVLYVHESIAEEFNKRFAEKVDGLAFGNPWEKGVSLTPLPETDKPKYIQGLIDDATHKGAKILNEKGGKHTDNYIFPAVLYPVNKEMRVYHEEQFGPVVPIISFKDINEPLEDMAESNYGQQVSLFGKDIKTLAPLIDALVNLVCRVNLNSSCQRGPDAFPFTGRKDSAVGTLSIPDALRSFSIRTFVASKDIAYNNEILQELLNSRESNFINTDYIL

SEQ ID NO: 77 - DNA, 1581 bp, synthetic construct
  source 1..1581: mol_type = other DNA
atgtctttgattccggaggaatttcaaataaaaaatttgataaaccaagatacttaccttgtaaatggggaacttaaaccctggacaggccaaacgacccctgtcttctctacgatttcctccacggaaaagtatactcccacattattaggcagtattcctttcatggaggaaaaagaagcagcggaagtagtagaagcagcgaacacagcatataacaaggggcagggactttggccaactatgaaagttgtagatcgtataaagtgcatggaaaattttgttaaccaaatgaaagagactagggaagaagttgtaaaattgctgatgtgggaaatagggaaaaatcttggtgactcccaaaaagagtttgatagaacggttgaatatatacaagatacaatcgatagctataaagagcttaacggaagatccagccactttgaaaaagttcagggcgttaacgccatgattaggaggggtccattaggggtcgttttatgcctgggtccatataattacccactaaatgagacgttttctttgttaataccggctttaattatgggcaacacagttatcttcaaaccggctaaacatggtgtattatgtattagccctcttcttgaggctttcagatcgagctttcccaagggagttattaacattgtgtatggtcgtgggagagaggtagcatctccgatcatgaagagcgggaaaatcgacgttttagcattaattggcaactcaaaatcagcaattgctttacaagatcaacatccaaacaagaatagattgagattaattttaggcctggaagccaaaaatccagctattattcttccagacgcagatcttgatttagcgatccaagagtgtatcgctggcacattatcgtttaacggacaaagatgtaccgctttgaaagtcctttatgtgcacgaatccattgctgaggagtttaacaagaggttcgccgaaaaggtggacggtttagcttttggcaatccatgggagaagggtgtttcactgacacctttacctgaaactgacaagccgaagtatatacaaggtctgattgacgatgccacccacaagggtgcaaagatcctaaatgaaaaaggtggtaagcatacagataattacattttcccagctgtactgtatcctgtgaataaggaaatgagagtatatcatgaggagcagtttggcccagttgtgcccataatttctttcaaggacattaatgagccacttgaagacatggctgaatcgaattatggtcaacaggtttcattgtttggtaaagacataaaaactttggcacctttaatcgacgctctagtaaatctagtgtgcagggtcaaccttaactcaagttgtcaaagaggacctgacgcatttccatttacaggtcgtaaggattccgctgtagggacattgtctataccggatgccttgaggtcatttagcattagaacttttgttgctagtaaggacatcgcatacaataatgaaatattgcaggaattattaaactctcgtgagagtaatttcattaacacagattatatactttaa

SEQ ID NO: 78 - DNA, 4055 bp, synthetic construct
  source 1..4055: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgactaaacagtataaaaatttggtcaacggggaatggaagctgtccgataatgatattaaaatatacgctcccgctaatggcgaagaactaggttccgttccggctatgagccaagaggacgtggattatgtttacgctaccgccaaggccgcgcagcctgcatggagggcattaagctatgtagaacgtgccgcttatttacataaggctgctgatatcttggtacgtgatgccgagaaaattggggctgttctgagtaaggaagtggctaagggttataaagcagctgtttctgaagttgttagaaccgcagatataatcagatatgccgccgaggaaggtgttagattagaaggagaagtactggaaggtggttcatttgaagcagggtctaagaagaaaatagctatagtcagaagagaacctttagggctggttcttgcgattagtccattcaactatccggttaatttagccggctccaaaatcgctccggccttgatttctggtaatgtcgttgctctaaagccaccgactcaaggttctatatcgggcctgttgttagccgaggcctttgcagaagctgggttaccggcgggtgtgttttctacaataacaggtaagggtagtgtaatcggtgattatatagttgaacatgaagccgttaattttataaatttcaccgggtctacccctataggagagagaattggtaaattggcagggatgcgtccgatcatgttagagctagggggaaaagactcagccattgtgctggaagatgctaatctggcaaaagctgcaaaagatatagttttaggtggtttatcctacagcggacagaggtgtacagcagtgaagagagtcttggttatggactcggttgcagacaaattgaccgaactgattaacagtgaagttgctaagctaagcgtcggtatgccagaagacaacgctgatattacaccagttatcgacagtggttctgctgattatattgaggggcttgcaaaagatgccgaagaaaagggagccaaggcccaaacggagtttaagagagaaggtaatcttatttggccaactatttacgataacgttactacagatatgaggttagcatgggaagagccatttggtccaattatgcctataattagggtttccagtgtagaggaagccgtaaagatatccaatgagtcggaatatggtttgcaagcatccgtttttactaacaattttccgctagcattcaagattgctgaacaattagaggtaggtacagttcacattaacaacaaaacacaaaggggcacagataatttcccgttcctaggtgctaagaaatcgggtgcaggcacccaaggagttaagtattcaatagaagctatgacaactgttaaaagcacggtgtttgatattgcagattaacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 79 - DNA, 3844 bp, synthetic construct
  source 1..3844: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttaatctgcaatatcaaacaccgtgcttttaacagttgtcatagcttctattgaatacttaactccttgggtgcctgcacccgatttcttagcacctaggaacgggaaattatctgtgcccctttgtgttttgttgttaatgtgaactgtacctacctctaattgttcagcaatcttgaatgctagcggaaaattgttagtaaaaacggatgcttgcaaaccatattccgactcattggatatctttacggcttcctctacactggaaaccctaattataggcataattggaccaaatggctcttcccatgctaacctcatatctgtagtaacgttatcgtaaatagttggccaaataagattaccttctctcttaaactccgtttgggccttggctcccttttcttcggcatcttttgcaagcccctcaatataatcagcagaaccactgtcgataactggtgtaatatcagcgttgtcttctggcataccgacgcttagcttagcaacttcactgttaatcagttcggtcaatttgtctgcaaccgagtccataaccaagactctcttcactgctgtacacctctgtccgctgtaggataaaccacctaaaactatatcttttgcagcttttgccagattagcatcttccagcacaatggctgagtcttttccccctagctctaacatgatcggacgcatccctgccaatttaccaattctctctcctataggggtagacccggtgaaatttataaaattaacggcttcatgttcaactatataatcaccgattacactacccttacctgttattgtagaaaacacacccgccggtaacccagcttctgcaaaggcctcggctaacaacaggcccgatatagaaccttgagtcggtggctttagagcaacgacattaccagaaatcaaggccggagcgattttggagccggctaaattaaccggatagttgaatggactaatcgcaagaaccagccctaaaggttctcttctgactatagctattttcttcttagaccctgcttcaaatgaaccaccttccagtacttctccttctaatctaacaccttcctcggcggcatatctgattatatctgcggttctaacaacttcagaaacagctgctttataacccttagccacttccttactcagaacagccccaattttctcggcatcacgtaccaagatatcagcagccttatgtaaataagcggcacgttctacatagcttaatgccctccatgcaggctgcgcggccttggcggtagcgtaaacataatccacgtcctcttggctcatagccggaacggaacctagttcttcgccattagcgggagcgtatattttaatatcattatcggacagcttccattccccgttgaccaaatttttatactgtttagtcatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 80 - protein, 476 aa, Streptococcus henryi
MTKQYKNLVNGEWKLSDNDIKIYAPANGEELGSVPAMSQEDVDYVYATAKAAQPAWRALSYVERAAYLHKAADILVRDAEKIGAVLSKEVAKGYKAAVSEVVRTADIIRYAAEEGVRLEGEVLEGGSFEAGSKKKIAIVRREPLGLVLAISPFNYPVNLAGSKIAPALISGNVVALKPPTQGSISGLLLAEAFAEAGLPAGVFSTITGKGSVIGDYIVEHEAVNFINFTGSTPIGERIGKLAGMRPIMLELGGKDSAIVLEDANLAKAAKDIVLGGLSYSGQRCTAVKRVLVMDSVADKLTELINSEVAKLSVGMPEDNADITPVIDSGSADYIEGLAKDAEEKGAKAQTEFKREGNLIWPTIYDNVTTDMRLAWEEPFGPIMPIIRVSSVEEAVKISNESEYGLQASVFTNNFPLAFKIAEQLEVGTVHINNKTQRGTDNFPFLGAKKSGAGTQGVKYSIEAMTTVKSTVFDIAD

SEQ ID NO: 81 - DNA, 1431 bp, synthetic construct
  source 1..1431: mol_type = other DNA
atgactaaacagtataaaaatttggtcaacggggaatggaagctgtccgataatgatattaaaatatacgctcccgctaatggcgaagaactaggttccgttccggctatgagccaagaggacgtggattatgtttacgctaccgccaaggccgcgcagcctgcatggagggcattaagctatgtagaacgtgccgcttatttacataaggctgctgatatcttggtacgtgatgccgagaaaattggggctgttctgagtaaggaagtggctaagggttataaagcagctgtttctgaagttgttagaaccgcagatataatcagatatgccgccgaggaaggtgttagattagaaggagaagtactggaaggtggttcatttgaagcagggtctaagaagaaaatagctatagtcagaagagaacctttagggctggttcttgcgattagtccattcaactatccggttaatttagccggctccaaaatcgctccggccttgatttctggtaatgtcgttgctctaaagccaccgactcaaggttctatatcgggcctgttgttagccgaggcctttgcagaagctgggttaccggcgggtgtgttttctacaataacaggtaagggtagtgtaatcggtgattatatagttgaacatgaagccgttaattttataaatttcaccgggtctacccctataggagagagaattggtaaattggcagggatgcgtccgatcatgttagagctagggggaaaagactcagccattgtgctggaagatgctaatctggcaaaagctgcaaaagatatagttttaggtggtttatcctacagcggacagaggtgtacagcagtgaagagagtcttggttatggactcggttgcagacaaattgaccgaactgattaacagtgaagttgctaagctaagcgtcggtatgccagaagacaacgctgatattacaccagttatcgacagtggttctgctgattatattgaggggcttgcaaaagatgccgaagaaaagggagccaaggcccaaacggagtttaagagagaaggtaatcttatttggccaactatttacgataacgttactacagatatgaggttagcatgggaagagccatttggtccaattatgcctataattagggtttccagtgtagaggaagccgtaaagatatccaatgagtcggaatatggtttgcaagcatccgtttttactaacaattttccgctagcattcaagattgctgaacaattagaggtaggtacagttcacattaacaacaaaacacaaaggggcacagataatttcccgttcctaggtgctaagaaatcgggtgcaggcacccaaggagttaagtattcaatagaagctatgacaactgttaaaagcacggtgtttgatattgcagattaa

SEQ ID NO: 82 - DNA, 4070 bp, synthetic construct
  source 1..4070: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgactgttaagcatgatcataccacacacccattccttatcaataacgaatggttggaaagcaagacgggtgagacagttcctatacattctccagacaatggcgttcaggtcggccaagtacccgctatgagtcgtgaagaggtaaaccaagctgttaacttggcggctgacgtacaggaaaattgggctgcgaccgaagtgcatgagagaagcgatttgcttcatagatgggctgacgagttggatcaagtggctgataagattggtgccatgatccaaaaggaagttggaaaaaccttcgcttcagctaagtctgaggtcaaaagaacggctcaattgattagacatacagccgaagaaggtctgagaacccatggatcttttatacagggtgatgcttttccaggtgccagtaggtcgaccaaagctatggttcaaaaagttcctcacggtgttattttagccatctcaccttttaactaccctgtaaatctggctgcctcaaagatcgctccggccttggttactggtaataccgttgtttttaaacctgctacacagggcgccgtttccggattgatgatgattgacgcccttgtcaaagccggacttccaaaaggcgttttaaatgtggtgactggtagaggttctgtcattggtgactacgtcgtaacccatccgaagttggatatgattacttttacgggtgggactgctaccggtcagcatattgccaaaaaagcaagcatgataccagtagttttagagctgggtggaaaagacccagctatagtactagaagatgccgacttaaagaaagcggcaaaagaaattgtatctggtgcgtttagttatagcgggcaaagatgtacggccataaagagagtgatggtaacggagcaagcagccgatgaattagtagagaaaattagggagcacgttgctgcgttatctgttggctacagtactgaggaagttgatattattcctatgattgaccagaagtctgcagactacgtaacaggtctgattgaggatgctgttaaaaagggtgcaacaattgtacacgaaggtcaaaatgaccaaaatttgttatcccccgttgtgttagaccacgttaccgaagatatggaaatagcctgggaggaacaatttggtccagttttacctatcatgagggttgcaaatgagctggaggcggttgatttggaaaaacgtaacgcctatggtttacaggcttcgatctttactaaggacatggaaaaggcttttaccttagcagagagaatgaatgtgggcaccgtacagattaatgggaagacttcccgtggtcccgatcatttcccattccttggagttaaaaattccgggcaaggtgttcagggtattggtagatccatagactccatgctaagggataaggttattgtattgaatctgtaacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 83 - DNA, 3859 bp, synthetic construct
  source 1..3859: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttacagattcaatacaataaccttatcccttagcatggagtctatggatctaccaataccctgaacaccttgcccggaatttttaactccaaggaatgggaaatgatcgggaccacgggaagtcttcccattaatctgtacggtgcccacattcattctctctgctaaggtaaaagccttttccatgtccttagtaaagatcgaagcctgtaaaccataggcgttacgtttttccaaatcaaccgcctccagctcatttgcaaccctcatgataggtaaaactggaccaaattgttcctcccaggctatttccatatcttcggtaacgtggtctaacacaacgggggataacaaattttggtcattttgaccttcgtgtacaattgttgcaccctttttaacagcatcctcaatcagacctgttacgtagtctgcagacttctggtcaatcataggaataatatcaacttcctcagtactgtagccaacagataacgcagcaacgtgctccctaattttctctactaattcatcggctgcttgctccgttaccatcactctctttatggccgtacatctttgcccgctataactaaacgcaccagatacaatttcttttgccgctttctttaagtcggcatcttctagtactatagctgggtcttttccacccagctctaaaactactggtatcatgcttgcttttttggcaatatgctgaccggtagcagtcccacccgtaaaagtaatcatatccaacttcggatgggttacgacgtagtcaccaatgacagaacctctaccagtcaccacatttaaaacgccttttggaagtccggctttgacaagggcgtcaatcatcatcaatccggaaacggcgccctgtgtagcaggtttaaaaacaacggtattaccagtaaccaaggccggagcgatctttgaggcagccagatttacagggtagttaaaaggtgagatggctaaaataacaccgtgaggaactttttgaaccatagctttggtcgacctactggcacctggaaaagcatcaccctgtataaaagatccatgggttctcagaccttcttcggctgtatgtctaatcaattgagccgttcttttgacctcagacttagctgaagcgaaggtttttccaacttccttttggatcatggcaccaatcttatcagccacttgatccaactcgtcagcccatctatgaagcaaatcgcttctctcatgcacttcggtcgcagcccaattttcctgtacgtcagccgccaagttaacagcttggtttacctcttcacgactcatagcgggtacttggccgacctgaacgccattgtctggagaatgtataggaactgtctcacccgtcttgctttccaaccattcgttattgataaggaatgggtgtgtggtatgatcatgcttaacagtcatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 84 - protein, 481 aa, Bacillus beveridgei
MTVKHDHTTHPFLINNEWLESKTGETVPIHSPDNGVQVGQVPAMSREEVNQAVNLAADVQENWAATEVHERSDLLHRWADELDQVADKIGAMIQKEVGKTFASAKSEVKRTAQLIRHTAEEGLRTHGSFIQGDAFPGASRSTKAMVQKVPHGVILAISPFNYPVNLAASKIAPALVTGNTVVFKPATQGAVSGLMMIDALVKAGLPKGVLNVVTGRGSVIGDYVVTHPKLDMITFTGGTATGQHIAKKASMIPVVLELGGKDPAIVLEDADLKKAAKEIVSGAFSYSGQRCTAIKRVMVTEQAADELVEKIREHVAALSVGYSTEEVDIIPMIDQKSADYVTGLIEDAVKKGATIVHEGQNDQNLLSPVVLDHVTEDMEIAWEEQFGPVLPIMRVANELEAVDLEKRNAYGLQASIFTKDMEKAFTLAERMNVGTVQINGKTSRGPDHFPFLGVKNSGQGVQGIGRSIDSMLRDKVIVLNL

SEQ ID NO: 85 - DNA, 1446 bp, synthetic construct
  source 1..1446: mol_type = other DNA
atgactgttaagcatgatcataccacacacccattccttatcaataacgaatggttggaaagcaagacgggtgagacagttcctatacattctccagacaatggcgttcaggtcggccaagtacccgctatgagtcgtgaagaggtaaaccaagctgttaacttggcggctgacgtacaggaaaattgggctgcgaccgaagtgcatgagagaagcgatttgcttcatagatgggctgacgagttggatcaagtggctgataagattggtgccatgatccaaaaggaagttggaaaaaccttcgcttcagctaagtctgaggtcaaaagaacggctcaattgattagacatacagccgaagaaggtctgagaacccatggatcttttatacagggtgatgcttttccaggtgccagtaggtcgaccaaagctatggttcaaaaagttcctcacggtgttattttagccatctcaccttttaactaccctgtaaatctggctgcctcaaagatcgctccggccttggttactggtaataccgttgtttttaaacctgctacacagggcgccgtttccggattgatgatgattgacgcccttgtcaaagccggacttccaaaaggcgttttaaatgtggtgactggtagaggttctgtcattggtgactacgtcgtaacccatccgaagttggatatgattacttttacgggtgggactgctaccggtcagcatattgccaaaaaagcaagcatgataccagtagttttagagctgggtggaaaagacccagctatagtactagaagatgccgacttaaagaaagcggcaaaagaaattgtatctggtgcgtttagttatagcgggcaaagatgtacggccataaagagagtgatggtaacggagcaagcagccgatgaattagtagagaaaattagggagcacgttgctgcgttatctgttggctacagtactgaggaagttgatattattcctatgattgaccagaagtctgcagactacgtaacaggtctgattgaggatgctgttaaaaagggtgcaacaattgtacacgaaggtcaaaatgaccaaaatttgttatcccccgttgtgttagaccacgttaccgaagatatggaaatagcctgggaggaacaatttggtccagttttacctatcatgagggttgcaaatgagctggaggcggttgatttggaaaaacgtaacgcctatggtttacaggcttcgatctttactaaggacatggaaaaggcttttaccttagcagagagaatgaatgtgggcaccgtacagattaatgggaagacttcccgtggtcccgatcatttcccattccttggagttaaaaattccgggcaaggtgttcagggtattggtagatccatagactccatgctaagggataaggttattgtattgaatctgtaa

SEQ ID NO: 86 - DNA, 4118 bp, synthetic construct
  source 1..4118: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatggctgctggtacaggtctttttgcggagattttggacggagatgcttataagtattacgctgacggtgtatggaaaaagagcgcttccggtaagagcgtttccattataaatcctacaacgaggaagacacaatacaaggttcaagcttgctcacaggaagaggttaataaagttatggatctagcaaagtctgcacaaaagctttgggctaaaacacctttgtggaaaagagccgaattattacacaaagcagctgctatcttaaaggagcataagacaccaattgctgagtgcttagttaaagaaatcgctaaaccagccaaggatgctgtcatggaagttgtccgttcaggtgacctagtttcatacactgctgaagaaggagttaggatcttaggtgagggtaaatttttggtgtccgattcattccccggtaacgagagaacaaaatactgtttgacctctaaaatccctttaggtgtgattttggcgatccctccttttaactacccagttaacttagctgtgtctaaaattgcccctgctcttattgcaggcaacagtattgtactaaaacccccaactcaaggagccgtttctgccctgcatatggttcattgctttcaccttgctggttttcctaaaggtctaattaactgtgtcacaggtaaagggagtgaaataggtgattttcttactatgcatcctggtgtgaattgtatttcttttactggtggagatactggtatttcgatttccaagaaggctggcatgattccattgcagatggagttgggaggtaaagatgcgtgtatcgtgttggaggatgctgacctggatctagtcgctgctaatattattaaaggtggtttctcatactccgggcagagatgcactgcggtaaaagtcgttttagtaatggagagtgtagcagatgcgttagttgaaaaggtcaaagccaaagttgctaaattaacggtcggtcctccagaagatgattgtgacatcacaccagtcgttagtgagtctagcgctaattttattgagggtctagtgttagatgctaaagaaaaaggtgctacgttctgtcaagaatataaacgtgaaggtaatctgatctggccacttttgttggataacgttagacccgacatgagaatcgcatgggaggaacctttcggtcccgttttaccagtaataagaattaatagtgtagaggaggggattcaccattgtaacgcatctaacttcggactacaaggttgtgttttcacaaaggatgtgaacaaagctattatgatttctgatgctatggaaaccggtacggtgcaaataaattccgcacctgctagaggaccagatcacttccccttccaaggaattaaagattctgggattgggtcacagggtataacaaattccataaatatgatgacaaaggtgaagactacagtcattaatttgccttccccatcatatacgatgggataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 87 - DNA, 3907 bp, synthetic construct
  source 1..3907: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttatcccatcgtatatgatggggaaggcaaattaatgactgtagtcttcacctttgtcatcatatttatggaatttgttataccctgtgacccaatcccagaatctttaattccttggaaggggaagtgatctggtcctctagcaggtgcggaatttatttgcaccgtaccggtttccatagcatcagaaatcataatagctttgttcacatcctttgtgaaaacacaaccttgtagtccgaagttagatgcgttacaatggtgaatcccctcctctacactattaattcttattactggtaaaacgggaccgaaaggttcctcccatgcgattctcatgtcgggtctaacgttatccaacaaaagtggccagatcagattaccttcacgtttatattcttgacagaacgtagcacctttttctttagcatctaacactagaccctcaataaaattagcgctagactcactaacgactggtgtgatgtcacaatcatcttctggaggaccgaccgttaatttagcaactttggctttgaccttttcaactaacgcatctgctacactctccattactaaaacgacttttaccgcagtgcatctctgcccggagtatgagaaaccacctttaataatattagcagcgactagatccaggtcagcatcctccaacacgatacacgcatctttacctcccaactccatctgcaatggaatcatgccagccttcttggaaatcgaaataccagtatctccaccagtaaaagaaatacaattcacaccaggatgcatagtaagaaaatcacctatttcactccctttacctgtgacacagttaattagacctttaggaaaaccagcaaggtgaaagcaatgaaccatatgcagggcagaaacggctccttgagttgggggttttagtacaatactgttgcctgcaataagagcaggggcaattttagacacagctaagttaactgggtagttaaaaggagggatcgccaaaatcacacctaaagggattttagaggtcaaacagtattttgttctctcgttaccggggaatgaatcggacaccaaaaatttaccctcacctaagatcctaactccttcttcagcagtgtatgaaactaggtcacctgaacggacaacttccatgacagcatccttggctggtttagcgatttctttaactaagcactcagcaattggtgtcttatgctcctttaagatagcagctgctttgtgtaataattcggctcttttccacaaaggtgttttagcccaaagcttttgtgcagactttgctagatccataactttattaacctcttcctgtgagcaagcttgaaccttgtattgtgtcttcctcgttgtaggatttataatggaaacgctcttaccggaagcgctctttttccatacaccgtcagcgtaatacttataagcatctccgtccaaaatctccgcaaaaagacctgtaccagcagccatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 88 - protein, 497 aa, Glycine soja
MAAGTGLFAEILDGDAYKYYADGVWKKSASGKSVSIINPTTRKTQYKVQACSQEEVNKVMDLAKSAQKLWAKTPLWKRAELLHKAAAILKEHKTPIAECLVKEIAKPAKDAVMEVVRSGDLVSYTAEEGVRILGEGKFLVSDSFPGNERTKYCLTSKIPLGVILAIPPFNYPVNLAVSKIAPALIAGNSIVLKPPTQGAVSALHMVHCFHLAGFPKGLINCVTGKGSEIGDFLTMHPGVNCISFTGGDTGISISKKAGMIPLQMELGGKDACIVLEDADLDLVAANIIKGGFSYSGQRCTAVKVVLVMESVADALVEKVKAKVAKLTVGPPEDDCDITPVVSESSANFIEGLVLDAKEKGATFCQEYKREGNLIWPLLLDNVRPDMRIAWEEPFGPVLPVIRINSVEEGIHHCNASNFGLQGCVFTKDVNKAIMISDAMETGTVQINSAPARGPDHFPFQGIKDSGIGSQGITNSINMMTKVKTTVINLPSPSYTMG

SEQ ID NO: 89 - DNA, 1494 bp, synthetic construct
  source 1..1494: mol_type = other DNA
atggctgctggtacaggtctttttgcggagattttggacggagatgcttataagtattacgctgacggtgtatggaaaaagagcgcttccggtaagagcgtttccattataaatcctacaacgaggaagacacaatacaaggttcaagcttgctcacaggaagaggttaataaagttatggatctagcaaagtctgcacaaaagctttgggctaaaacacctttgtggaaaagagccgaattattacacaaagcagctgctatcttaaaggagcataagacaccaattgctgagtgcttagttaaagaaatcgctaaaccagccaaggatgctgtcatggaagttgtccgttcaggtgacctagtttcatacactgctgaagaaggagttaggatcttaggtgagggtaaatttttggtgtccgattcattccccggtaacgagagaacaaaatactgtttgacctctaaaatccctttaggtgtgattttggcgatccctccttttaactacccagttaacttagctgtgtctaaaattgcccctgctcttattgcaggcaacagtattgtactaaaacccccaactcaaggagccgtttctgccctgcatatggttcattgctttcaccttgctggttttcctaaaggtctaattaactgtgtcacaggtaaagggagtgaaataggtgattttcttactatgcatcctggtgtgaattgtatttcttttactggtggagatactggtatttcgatttccaagaaggctggcatgattccattgcagatggagttgggaggtaaagatgcgtgtatcgtgttggaggatgctgacctggatctagtcgctgctaatattattaaaggtggtttctcatactccgggcagagatgcactgcggtaaaagtcgttttagtaatggagagtgtagcagatgcgttagttgaaaaggtcaaagccaaagttgctaaattaacggtcggtcctccagaagatgattgtgacatcacaccagtcgttagtgagtctagcgctaattttattgagggtctagtgttagatgctaaagaaaaaggtgctacgttctgtcaagaatataaacgtgaaggtaatctgatctggccacttttgttggataacgttagacccgacatgagaatcgcatgggaggaacctttcggtcccgttttaccagtaataagaattaatagtgtagaggaggggattcaccattgtaacgcatctaacttcggactacaaggttgtgttttcacaaaggatgtgaacaaagctattatgatttctgatgctatggaaaccggtacggtgcaaataaattccgcacctgctagaggaccagatcacttccccttccaaggaattaaagattctgggattgggtcacagggtataacaaattccataaatatgatgacaaaggtgaagactacagtcattaatttgccttccccatcatatacgatgggataa

SEQ ID NO: 90 - DNA, 4055 bp, synthetic construct
  source 1..4055: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgacagaacactatttaaactatgtcaacggagagtggagagattctgctgatgctattgaaatctttgaacccgctacgggaaagtcgctaggtaccgtccctgccatgtcgcatgaagacgttgattacgtaatggattccgcaaaaaaggcattgcctgcttggcgtgctttatcctacgttgaacgtgctgcctatttgcaaaaagctgctgatattctatatagggatgccgaaaaaattgggagtactttatccaaggaaattgctaagggcttgaaatcgtcaataggtgaagtcaccaggacagctgaaattgttgagtatacagcaaaggtaggtgtgactttagatggagaagtcatggaaggtgggaatttcgaggctgcatcaaagaacaagttagccgttgtgagacgtgagcctgttggtttggtcctagctatctcccctttcaattatccagttaatctagccggtagtaaaatagcaccggcattaatgggcgggaatgttgtggcctttaaacccccaactcagggttcaatttctggactactattagcgaaggcattcgcggaggctggccttcctgccggggtttttaatacaataacaggaagaggccgtgtcatcggtgattatattgtggaacatagcgctgtaaatttcatcaacttcaccggctcaagtgccgtgggaaaaaacatcggcaaacttgcaggtatgaggcctattatgcttgaattgggtggaaaggacgctgctatagtattggaggacgcagacttagatttaacagcaaaaaacattgtagctggcgctttcggatacagcggtcaaaggtgtacggctgttaagagagtgttggtcatggattcggtcgcagacgaattagtagaaaaggtaaccgcgctagccaaagatttgacggtaggcatgcctgaagaggatgctgatataactccattgattaacactaagtccgccgactatgtacaaggattgatcgaagaggctgccgaaaagggtgcgaagccgttgtccgattttaagagagaaggaaacttaatttaccctattgtaatggatcaagtgaccactgatatgagattagcctgggaggagccttttgggccagttttgccctttataagggtgaaaagtgctgatgaggcagttaagatcgccaacgagagcgaatatggattgcaatcgtcggttttcagtagaaatttcgagaaggctttcgctattgccggtaagctagaagtgggaacagtacatatcaacaataagacgcagagaggtccagataattttccatttttgggggtaaaaagttcaggcgcaggggttcaaggagttaagtactcaatccaggctatgacaagggttaagtcagtcgtgtttaacatcgaaggttaacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 91 - DNA, 3844 bp, synthetic construct
  source 1..3844: mol_type = other DNA
gcggccgcatcgtccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagatgcattgggtcaacagtatagaaccgtggatgatgtcgtgtctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatgtcacagtatcggcatctctgtcagcataaaggcattaaaagaggagcgaatttttttttaataaaaatcttaataatcattaaaagataaataatagtctatatatacgtatataaataaaaaatattcaaaaaataaaataaactattattttagcgtaaaggatggggaaagagaaaagaaaaaaattgatctatcgatttcaattcaattcaatgttaaccttcgatgttaaacacgactgacttaacccttgtcatagcctggattgagtacttaactccttgaacccctgcgcctgaactttttacccccaaaaatggaaaattatctggacctctctgcgtcttattgttgatatgtactgttcccacttctagcttaccggcaatagcgaaagccttctcgaaatttctactgaaaaccgacgattgcaatccatattcgctctcgttggcgatcttaactgcctcatcagcacttttcacccttataaagggcaaaactggcccaaaaggctcctcccaggctaatctcatatcagtggtcacttgatccattacaatagggtaaattaagtttccttctctcttaaaatcggacaacggcttcgcacccttttcggcagcctcttcgatcaatccttgtacatagtcggcggacttagtgttaatcaatggagttatatcagcatcctcttcaggcatgcctaccgtcaaatctttggctagcgcggttaccttttctactaattcgtctgcgaccgaatccatgaccaacactctcttaacagccgtacacctttgaccgctgtatccgaaagcgccagctacaatgttttttgctgttaaatctaagtctgcgtcctccaatactatagcagcgtcctttccacccaattcaagcataataggcctcatacctgcaagtttgccgatgttttttcccacggcacttgagccggtgaagttgatgaaatttacagcgctatgttccacaatataatcaccgatgacacggcctcttcctgttattgtattaaaaaccccggcaggaaggccagcctccgcgaatgccttcgctaatagtagtccagaaattgaaccctgagttgggggtttaaaggccacaacattcccgcccattaatgccggtgctattttactaccggctagattaactggataattgaaaggggagatagctaggaccaaaccaacaggctcacgtctcacaacggctaacttgttctttgatgcagcctcgaaattcccaccttccatgacttctccatctaaagtcacacctacctttgctgtatactcaacaatttcagctgtcctggtgacttcacctattgacgatttcaagcccttagcaatttccttggataaagtactcccaattttttcggcatccctatatagaatatcagcagctttttgcaaataggcagcacgttcaacgtaggataaagcacgccaagcaggcaatgccttttttgcggaatccattacgtaatcaacgtcttcatgcgacatggcagggacggtacctagcgactttcccgtagcgggttcaaagatttcaatagcatcagcagaatctctccactctccgttgacatagtttaaatagtgttctgtcatttttgtttgtttatgtgtgtttattcgaaactaagttcttggtgttttaaactaaaaaaaagactaactataaaagtagaatttaagaagtttaagaaatagatttacagaattacaatcaatacctaccgtctttatatacttattagtcaaataggggaataatttcagggaactggtttcaaccttttttttcagctttttccaaatcagagagagcagaaggtaatagaaggtgtaagaaaatgagatagatacatgcgtaggtcaattgccttgtgtcatcatttactccaagcaggttgcatcactccattgaggttgtgcccgttttttgcctgtttgtgcccctgttctgtgtagttgcgctaagagaatggacctatgaactgatggttggtgaagaaaacaatattttggtgctgggattctttttttttctggatgccagcttaaaaaaagcgggctccattatatttagtggatgccaggaataaactgttcacccagacacctatgatgttatatattctgtgtaacccgccccctattttgggcatgtacgggttacagcagaattaaaaggccaattttttgactaaataaagttaggaaaatcactactattaattatttacgtattctttgaaatggcagtattgataatgataaactcggcttgaagactgagtccgtctcagcgagctcttctagattttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgtacaacgcggccgc

SEQ ID NO: 92 - protein, 476 aa, Lactobacillus delbrueckii
MTEHYLNYVNGEWRDSADAIEIFEPATGKSLGTVPAMSHEDVDYVMDSAKKALPAWRALSYVERAAYLQKAADILYRDAEKIGSTLSKEIAKGLKSSIGEVTRTAEIVEYTAKVGVTLDGEVMEGGNFEAASKNKLAVVRREPVGLVLAISPFNYPVNLAGSKIAPALMGGNVVAFKPPTQGSISGLLLAKAFAEAGLPAGVFNTITGRGRVIGDYIVEHSAVNFINFTGSSAVGKNIGKLAGMRPIMLELGGKDAAIVLEDADLDLTAKNIVAGAFGYSGQRCTAVKRVLVMDSVADELVEKVTALAKDLTVGMPEEDADITPLINTKSADYVQGLIEEAAEKGAKPLSDFKREGNLIYPIVMDQVTTDMRLAWEEPFGPVLPFIRVKSADEAVKIANESEYGLQSSVFSRNFEKAFAIAGKLEVGTVHINNKTQRGPDNFPFLGVKSSGAGVQGVKYSIQAMTRVKSVVFNIEG

SEQ ID NO: 93 - DNA, 1431 bp, synthetic construct
  source 1..1431: mol_type = other DNA
atgacagaacactatttaaactatgtcaacggagagtggagagattctgctgatgctattgaaatctttgaacccgctacgggaaagtcgctaggtaccgtccctgccatgtcgcatgaagacgttgattacgtaatggattccgcaaaaaaggcattgcctgcttggcgtgctttatcctacgttgaacgtgctgcctatttgcaaaaagctgctgatattctatatagggatgccgaaaaaattgggagtactttatccaaggaaattgctaagggcttgaaatcgtcaataggtgaagtcaccaggacagctgaaattgttgagtatacagcaaaggtaggtgtgactttagatggagaagtcatggaaggtgggaatttcgaggctgcatcaaagaacaagttagccgttgtgagacgtgagcctgttggtttggtcctagctatctcccctttcaattatccagttaatctagccggtagtaaaatagcaccggcattaatgggcgggaatgttgtggcctttaaacccccaactcagggttcaatttctggactactattagcgaaggcattcgcggaggctggccttcctgccggggtttttaatacaataacaggaagaggccgtgtcatcggtgattatattgtggaacatagcgctgtaaatttcatcaacttcaccggctcaagtgccgtgggaaaaaacatcggcaaacttgcaggtatgaggcctattatgcttgaattgggtggaaaggacgctgctatagtattggaggacgcagacttagatttaacagcaaaaaacattgtagctggcgctttcggatacagcggtcaaaggtgtacggctgttaagagagtgttggtcatggattcggtcgcagacgaattagtagaaaaggtaaccgcgctagccaaagatttgacggtaggcatgcctgaagaggatgctgatataactccattgattaacactaagtccgccgactatgtacaaggattgatcgaagaggctgccgaaaagggtgcgaagccgttgtccgattttaagagagaaggaaacttaatttaccctattgtaatggatcaagtgaccactgatatgagattagcctgggaggagccttttgggccagttttgccctttataagggtgaaaagtgctgatgaggcagttaagatcgccaacgagagcgaatatggattgcaatcgtcggttttcagtagaaatttcgagaaggctttcgctattgccggtaagctagaagtgggaacagtacatatcaacaataagacgcagagaggtccagataattttccatttttgggggtaaaaagttcaggcgcaggggttcaaggagttaagtactcaatccaggctatgacaagggttaagtcagtcgtgtttaacatcgaaggttaa

SEQ ID NO: 94 - DNA, 4073 bp, synthetic construct
  source 1..4073: mol_type = other DNA
gcggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgacaacatcaaatacctacaaattctatctaaacggtgaatggagagaatcttcctctggagaaactattgagataccatcaccatacttacatgaagtgatcggacaggttcaagcaatcactagaggagaggttgacgaagcgattgctagcgctaaggaagcacagaaatcttgggctgaggcatctctacaagatagagctaagtacttgtacaaatgggcagatgaattggtaaacatgcaagacgaaatcgccgatatcatcatgaaggaagtgggcaagggttacaaagacgctaaaaaggaggttgttagaaccgccgatttcatcagatacaccattgaagaggcactccatatgcacggtgaatccatgatgggcgattcatttcctggtggaacaaaatctaagctagcaataatccaaagagcgcctctgggtgtagtcttagccatcgctccattcaattaccctgtaaacctttctgctgcaaaattggcaccagccttaattatgggtaacgctgtgatattcaagccagcaactcagggtgctatttccggcatcaaaatggttgaagctttgcataaggctggtttgccaaagggtttggttaacgttgccacaggtagaggtagcgtcataggcgattatttggtcgaacacgaagggataaacatggtttccttcaccggtggcactaacactggtaagcatttagcaaaaaaggcctcaatgattccattagtcttggaacttggtggcaaagatccaggcatcgttcgtgaagatgcagacctacaagatgctgcgaatcatatcgtatctggtgcgttcagttactcagggcagagatgtacagccattaagagagtccttgttcatgaaaatgttgctgatgaactggtatcattggttaaggaacaagtggcaaagctttctgtgggatcaccagagcaagattcaacaattgttcctctgattgacgataagtccgctgattttgttcagggtttagtggacgatgcagtcgaaaagggcgctacaattgtcattgggaacaagagagaacgtaacctaatctacccaacattgattgatcacgtcacagaggaaatgaaagttgcctgggaggaaccattcggtcctattcttccaattattagagttagtagcgacgagcaagctattgaaattgcaaataagagtgagttcggattacaagcttctgtgtttaccaaagacataaacaaggcattcgcaatcgcaaataagattgagactggttcagtgcaaatcaacggtagaacagagagaggaccagatcactttccttttatcggggttaagggatctgggatgggtgcccaaggcatcagaaagtctttggaatctatgactagagaaaaagttactgtcttaaatctcgtatgataataataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgcggccgc

SEQ ID NO: 95 - DNA, 3862 bp, synthetic construct
  source 1..3862: mol_type = other DNA
gcggccgcgttgtacggtggttccagatgtgccaactgtgtcaaggaaagaatcgtcagagctttcttgattgaagaacaaaagattgttaagaaggtcgttaaggaacaaactgaggctgctaagaagtctgaaaagaagtctaagaaataaatttaaataagcattccttttataattactctaacttctcgtcgtactaaaaaaaaaacacttttatagtaagatataaaacaaaaacgaagattgaatcttccttttttatttctgtgttaaattctctaaaccagtttgatttgcgcgaaccacctgtcacggcagatcatccgaagacaacaaagaaaaggatgcattacatcggtttaaatttttctattcgtattttataagtattatattatattattaagtcatgcagatataaaattatacaattggtcggaaatggagggaaatcatacatttttattttatttttagcgtagtagttttatcaaaaaaataaaagaaaatctagaagagctcgctgagacggactcagtcttcaagccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgacaacatcaaatacctacaaattctatctaaacggtgaatggagagaatcttcctctggagaaactattgagataccatcaccatacttacatgaagtgatcggacaggttcaagcaatcactagaggagaggttgacgaagcgattgctagcgctaaggaagcacagaaatcttgggctgaggcatctctacaagatagagctaagtacttgtacaaatgggcagatgaattggtaaacatgcaagacgaaatcgccgatatcatcatgaaggaagtgggcaagggttacaaagacgctaaaaaggaggttgttagaaccgccgatttcatcagatacaccattgaagaggcactccatatgcacggtgaatccatgatgggcgattcatttcctggtggaacaaaatctaagctagcaataatccaaagagcgcctctgggtgtagtcttagccatcgctccattcaattaccctgtaaacctttctgctgcaaaattggcaccagccttaattatgggtaacgctgtgatattcaagccagcaactcagggtgctatttccggcatcaaaatggttgaagctttgcataaggctggtttgccaaagggtttggttaacgttgccacaggtagaggtagcgtcataggcgattatttggtcgaacacgaagggataaacatggtttccttcaccggtggcactaacactggtaagcatttagcaaaaaaggcctcaatgattccattagtcttggaacttggtggcaaagatccaggcatcgttcgtgaagatgcagacctacaagatgctgcgaatcatatcgtatctggtgcgttcagttactcagggcagagatgtacagccattaagagagtccttgttcatgaaaatgttgctgatgaactggtatcattggttaaggaacaagtggcaaagctttctgtgggatcaccagagcaagattcaacaattgttcctctgattgacgataagtccgctgattttgttcagggtttagtggacgatgcagtcgaaaagggcgctacaattgtcattgggaacaagagagaacgtaacctaatctacccaacattgattgatcacgtcacagaggaaatgaaagttgcctgggaggaaccattcggtcctattcttccaattattagagttagtagcgacgagcaagctattgaaattgcaaataagagtgagttcggattacaagcttctgtgtttaccaaagacataaacaaggcattcgcaatcgcaaataagattgagactggttcagtgcaaatcaacggtagaacagagagaggaccagatcactttccttttatcggggttaagggatctgggatgggtgcccaaggcatcagaaagtctttggaatctatgactagagaaaaagttactgtcttaaatctcgtatgataataataacattgaattgaattgaaatcgatagatcaatttttttcttttctctttccccatcctttacgctaaaataatagtttattttattttttgaatattttttatttatatacgtatatatagactattatttatcttttaatgattattaagatttttattaaaaaaaaattcgctcctcttttaatgcctttatgctgacagagatgccgatactgtgacataacttcgtatagcatacattatacgaagttatcaaacgcatgaaatccttcatttgcttttgttccactactttttggaactcttgttgttctttggagttcaatgcgtccatctttacagtcctgtcttattgttcttgatttgtgccccgtaaaatactgttacttggttctggcgaggtattggatggttcctttttataaaggccatgaagctttttctttccaattttttttttttcgtcattataaaaatcattacgaccgagattcccgggtaataactgatataattaaattgaagctctaatttgtgagtttagtatacatgcatttacttataatacagttttttagttttgctggccgcatcttctcaaatatgcttcccagcctgcttttctgtaacgttcaccctctaccttagcatcccttccctttgcaaatagtcctcttccaacaataataatgtcagatcctgtagacacgacatcatccacggttctatactgttgacccaatgcatctcccttgtcatctaaacccacaccgggtgtcataatcaaccaatcgtaaccttcatctcttccacccatgtctctttgagcaataaagccgataacaaaatctttgtcgctcttcgcaatgtcaacagtacccttagtatattctccagtagctagggagcccttgcatgacaattctgctaacatcaaaaggcctctaggttcctttgttacttcttccgccgcctgcttcaaaccgctaacaatacctgggcccaccacaccgtgtgcattcgtaatgtctgcccattctgctattctgtatacacccgcagagtactgcaatttgactgtattaccaatgtcagcaaattttctgtcttcgaagagtaaaaaattgtacttggcggataatgcctttagcggcttaactgtgccctccatggacgatgcggccgc

SEQ ID NO: 96 - protein, 479 aa, Bacillus cereus
MTTSNTYKFYLNGEWRESSSGETIEIPSPYLHEVIGQVQAITRGEVDEAIASAKEAQKSWAEASLQDRAKYLYKWADELVNMQDEIADIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKAGLPKGLVNVATGRGSVIGDYLVEHEGINMVSFTGGTNTGKHLAKKASMIPLVLELGGKDPGIVREDADLQDAANHIVSGAFSYSGQRCTAIKRVLVHENVADELVSLLKAQVAELSVGSPEQDSTIVPLIDDKSADFVQGLVDDAVENGATIVIGNKRERNLIYPTLIDHVTEEMKVAWEEPFGPILPIIRVSSDEQAIEIANKSEFGLQASVFTKDINKAFAIANKIETGSVQINGRTERGPDHFPFIGVKGSGMGAQGIRKSLESMTREKVTVLNLV

SEQ ID NO: 97 - DNA, 1440 bp, synthetic construct
  source 1..1440: mol_type = other DNA
atgactactagcaacacgtataagttttatcttaatggggagtggcgtgaatcctcatcaggggaaacgattgaaatcccatctccttatttgcacgaagtcatcggacaagttcaggcgataacaagaggtgaagtagacgaggcgatagcttccgctaaagaagcacagaaaagttgggccgaggcgagtttgcaagatcgtgcaaagtacttatataaatgggcagatgaattagtgaacatgcaggacgaaatagctgacattatcatgaaagaagttggaaagggttacaaggacgctaaaaaggaagttgtcagaaccgcagacttcattcgttacacaattgaggaggcactacatatgcatggcgaatcaatgatgggtgattccttccctggcggtacaaagtcaaaattggccattattcaaagggcaccacttggtgttgtacttgccatagctccattcaactacccagtaaacctatccgcagcaaagttagctcctgcactaataatgggaaatgccgttatcttcaaaccggcaacccaaggtgctatttccggtataaagatggtcgaagctttgcataaagcgggcctaccaaaaggtttggttaatgtggctactggcaggggttcagtaattggtgattaccttgttgaacatgaggggatcaatatggtcagttttactgggggaacaaatacaggtaaacatcttgctaagaaagcatcgatgatcccactggtcttggaactgggcggaaaggatccgggtatagttagagaagatgctgatttgcaggacgctgcaaaccatatagtttctggagctttcagttattcgggtcagcgttgtacagctattaaacgtgtattggtacatgagaatgtagccgacgaattggtttctcttttgaaagcacaagtcgcagagttatccgtaggttctcctgaacaggattctactattgtaccgctaatcgatgacaagtcggctgatttcgtccaagggctggtagacgatgccgttgagaacggcgccaccatcgtcattggtaataaaagagagagaaacttaatatatccaacattaatagatcatgtaacagaggagatgaaggttgcttgggaggaaccattcggtccaattctacctatcataagagtttcaagcgatgagcaagctattgaaatagctaataaatctgaattcggtttgcaagctagtgtctttacaaaggatataaataaggcgtttgcgattgctaacaagatcgaaacgggttcagtacaaattaacggtagaaccgagagaggtcccgatcatttcccatttattggtgttaaaggatcaggtatgggcgcgcaggggatcaggaagtcgttggagtcgatgaccagagaaaaagttacggttctaaacctagtttaa

SEQ ID NO: 98 - protein, 341 aa, Candida maltosa
MTLSTKPKVLKLGDFEFSQARWDELSQIAEVINCESQNRDEFINDLQTKYNDITYIAREYNSVNVTGRFDAEIAQHIPSTVKGICHCTAGYDSIDVEPFSKLGIQISNVTDPVVGPTADVAVFLVLACLRNFFAGHKMLVSGKWPDEKVLECGGVDMGNTPQHKVLGILGMGNIGRAVKNRLKVFGFKKIVYYNRRQLSPELEEGAEYVSQDELLQKSDVIVVVVPLNPNTKHLIDRNAISKMKDGVILVNISRGLVLDEHVVTELVKSGKIGAFGADVFENEPHVSPDLYNLPNVVTLPHMGTHAFDATQDLQEWVIENIECCIKTGKLKTLVAEQKELF

SEQ ID NO: 99 - DNA, 1026 bp, synthetic construct
  source 1..1026: mol_type = other DNA
atgacgctatctaccaagccgaaagtcttgaagctaggtgacttcgagttcagtcaagcaaggtgggatgaactgtcccagattgccgaggtcatcaactgcgaatctcagaatagggatgaattcatcaacgatttgcaaaccaaatacaacgatataacctatattgccagggaatataacagcgttaatgttactggaaggttcgacgcagagatagcacaacatatccccagcactgttaaggggatctgccattgcaccgctggttacgactctatcgatgttgagcccttcagcaagttggggattcagattagtaatgttacggatccagtggtaggacctactgctgacgttgcagtattccttgtgttagcttgtcttaggaatttctttgcaggtcataaaatgttggtatccgggaaatggcctgatgagaaggttcttgagtgtggcggagttgacatgggtaacactcctcaacataaggtactaggaatcttaggtatgggaaacattgggcgtgccgtgaagaataggttgaaagtattcggattcaagaagatcgtatactataatagaaggcaactgtcgccagagctggaagagggcgccgagtacgtaagtcaggacgagttgttgcagaaatccgatgttattgtcgtcgttgttcccttaaatccaaacacgaagcatctaatagaccgtaacgcgatatctaagatgaaggacggagtaatattggtgaacatttctcgtggattggtcttggatgaacacgttgtcacggagttagtgaaatccggcaagataggtgcctttggtgcagacgttttcgagaacgagccgcacgtctcacctgacctttacaacctgcctaacgtggtaacattgccgcacatggggacgcacgcgtttgacgcaactcaggacttgcaggagtgggtgattgagaatattgaatgctgtataaagactggaaaactgaaaactctggtcgcggaacagaaggagctgttctaa

SEQ ID NO: 100 - protein, 357 aa, Rhodotorula toruloides
MSTAPAQYTGYAALTEEKGKKLELEKWSYTPKQWTEDDVDIKVDCCGMCGSCIHTLTNGWPQPTSYPCIAGHEVVGTIVRAGSRTSHKVGTRVGVGAQGGSCLECDYCKQGLQQHCEKGLIMTYQGTWEDGSVAQGGYADFMRCRGALAVPIPDGLESETTAPLLCAGVTVYAPLKRFNAGPGKRVGVIGIGGLGHLALQIAKAMGAEVYALSHSASKMDDAEKLGCPRENFIVAKDQDEVRKKYNKHFDILILTSSSNDIPLEGLYLPLLRNMGTFVICGLPEEKLPSMYGQMLVYKSINLAGSLIGGSSEIAELFDLALKHDIKAWVETRPMSEVTQTAHDMHAGKSRYRYVLKN

SEQ ID NO: 101 - DNA, 1074 bp, synthetic construct
  source 1..1074: mol_type = other DNA
atgtcaactgctcctgcacaatacactggctacgctgccctgaccgaggagaaaggaaagaagcttgagcttgagaagtggtcatacactccaaagcagtggactgaagatgacgtggatattaaagttgattgctgcggcatgtgtggttcctgtatccatactcttaccaacggttggcctcaacctacatcttacccatgtatcgctgggcatgaggttgtggggacaattgtaagagctgggtcaaggactagccacaaggttggaactagggtcggtgttggtgctcaaggtggaagctgcttagagtgcgactattgcaagcagggcttgcagcagcattgcgagaaaggattgataatgacgtatcagggtacctgggaagacgggtccgtcgcacagggtggttacgctgattttatgagatgccgtggcgctttagctgtacctatacctgacggcttggagtctgagactaccgcaccgttgctgtgcgctggcgttaccgtctacgcgcctttaaagaggtttaacgccggtcccggtaagagagtgggtgtgatcgggattggcggtctgggacaccttgcgctgcaaattgcaaaagcaatgggagctgaggtatatgctttgtctcacagcgcgagcaaaatggacgacgccgagaagcttggatgccctagagagaactttatagtggctaaagaccaagacgaggtcaggaagaagtacaacaagcactttgacatattgatactaacatcttcttccaatgatatacccctagagggattgtacctgccattattacgtaacatgggcactttcgtgatctgcggattacctgaagagaagttgccgtctatgtatggacagatgttggtctacaaatcgataaacttggctggatcattgatcggcgggagttcagaaattgctgagctattcgacttagcacttaagcatgacatcaaggcctgggttgagacaagaccaatgtctgaagttacacaaaccgctcacgatatgcatgcgggaaagtcgcgttaccgttacgttctgaagaattaa

SEQ ID NO: 102 - DNA, 6000 bp, synthetic construct
  source 1..6000: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatggccaagcaatataagaacttggttaatggtgaatggaagttgtctgaaaacgagatcaccatttatgcacctgctactggtgaagaacttggcagcgtccccgccatgactcaagccgaagtagatgccgtttatgcatccgccaagaaggccttatcagactggagagctctgtcatatgttgaaagggcagcttatttgcataaggctgcagatattttggttcgtgacgcagagaagattggtgcaatcttatcaaaagaagtagcaaaaggtcataaagctgcagtttctgaagtgattagaacagctgaaataattaattacgccgcagaagaaggtcttaggatggaaggagaagttcttgaaggcggaagcttcgaagccgcatctaaaaaaaagatcgctattgtcagaagagaaccagtaggtctagtgctagcaatatctccttttaactaccctgtaaatttagcaggcagcaagatagcaccagctttaatagcaggcaatgtagtggctttgaaaccaccaacacagggctcaataagcggtctgctgttagccgaggcttttgctgaagctggtattcctgcaggtgtatttaatacaataactgggagaggttctgttattggtgactatattgttgagcacgaggctgtaaatttcataaattttactggttctactcctattggcgagggtatcggaaagctagcaggtatgaggcctatcatgttagagctgggtggaaaagattctgccatcgtattggaagacgctgatcttgctttggctgcaaaaaacattgtagctggagcatttggttacagtggtcaaagatgcaccgccgtgaagagagttctggttatggataaagtagcagaccaattagctgctgaaatcaagacattggttgaaaagttgtcagtgggtatgccagaagacgacgctgatattactccgcttattgatacaagtgccgccgatttcgtagaaggtcttattaaagacgccacggacaaaggtgcaacagcattaacagcgtttaatagagaaggaaatttaatctcgcctgtgttattcgatcatgtaactacagatatgagattggcctgggaggaaccttttgggcctgtcttaccaattattcgtgttactaccgttgaagaggcaataaaaatttctaatgaaagtgagtacgggttgcaagcttcaattttcactactaattttcctaaagcctttggcattgcagaacaattggaagtaggcactgtacacctgaataacaaaacccaaagaggcaccgataatttcccatttcttggtgctaaaaagtcaggtgctggagttcaaggtgtaaaatattccattgaagctatgacaacagtgaagagtgttgttttcgacatacaataacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgacgctatctaccaagccgaaagtcttgaagctaggtgacttcgagttcagtcaagcaaggtgggatgaactgtcccagattgccgaggtcatcaactgcgaatctcagaatagggatgaattcatcaacgatttgcaaaccaaatacaacgatataacctatattgccagggaatataacagcgttaatgttactggaaggttcgacgcagagatagcacaacatatccccagcactgttaaggggatctgccattgcaccgctggttacgactctatcgatgttgagcccttcagcaagttggggattcagattagtaatgttacggatccagtggtaggacctactgctgacgttgcagtattccttgtgttagcttgtcttaggaatttctttgcaggtcataaaatgttggtatccgggaaatggcctgatgagaaggttcttgagtgtggcggagttgacatgggtaacactcctcaacataaggtactaggaatcttaggtatgggaaacattgggcgtgccgtgaagaataggttgaaagtattcggattcaagaagatcgtatactataatagaaggcaactgtcgccagagctggaagagggcgccgagtacgtaagtcaggacgagttgttgcagaaatccgatgttattgtcgtcgttgttcccttaaatccaaacacgaagcatctaatagaccgtaacgcgatatctaagatgaaggacggagtaatattggtgaacatttctcgtggattggtcttggatgaacacgttgtcacggagttagtgaaatccggcaagataggtgcctttggtgcagacgttttcgagaacgagccgcacgtctcacctgacctttacaacctgcctaacgtggtaacattgccgcacatggggacgcacgcgtttgacgcaactcaggacttgcaggagtgggtgattgagaatattgaatgctgtataaagactggaaaactgaaaactctggtcgcggaacagaaggagctgttctaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacga

SEQ ID NO: 103 - DNA, 6272 bp, synthetic construct
  source 1..6272: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatgtcatccgaaaataccgcaagttttcaccatatcttcaaatcagagaacgaaatccctgaagaatataaagttccagttatacaccagcgtacgtatctgttgaacggggaattagtggaatggaacggggacgtgactgaaatctattcaccagtttgtattccaacagaaaatggcttggaaaggaaattattgggaagtattccaaatataggacctaatgaagctatggaagttcttgaggcttgcgtaaaagcatacgataatgggttgggagaatggccgactatgtcagtggaagggagaattaagtgtatgcaaaagttcgtttatctgatgatacaacaaagagatttaattatcaaactattgatgtgggagattggtaagactttggctgattctacaaaagagtttgataggacggtcgattatattaatcaaacaattgacgccttgaaggatctagatagagaatcttctcgttttcaacaggccgaaggtaccatcgcccaaattaggagagcgcctttgggagtcgtgctgagtatgggtcccttcaattatccgttgaatgaaatcttcacaactttaatccctgcattaataatgggtaatacaattttattcaagttacctaagcatggtgttttggcccattacccccttctagaagcatttaaagaagcttttccaaagggaaccgtaaacacgttgtacggtaagggcagtgaaattataactccaattatggagtccgggaaagtaaacgtgcttgcctttattggttccagcaaggtagctaatggactaaaaaaactacatccaaaggtgaataggctgagagcaattctgagcctagatgcaaagaatgcagcaattgtcacaaagaacgctaatttggacgtggcagtttcagaatgtatattaggagctcttagtttcaatggccagcgttgtactgcattaaaacttatctttgtccagaaggaggttgcactggaatttaccgaaaagttatcagcggctgtgtcggcattgaagcctggattaccttgggaaaaagatgttaaagtaacacctctaccagaagttaataagcctccatatttgaaagaatgcatagatgatgctttgcagaagggagccgcagttttaaataaagatggtgggtataccgatgaatcttttgtatttcccgccgttgtataccccgttaattctgatatgaagctgtatcacgaagaacaatttggtccagtaataccggtagtgccctttgaagatatcgaggagcccattgagtatcaagtcaatgcctctcatggtatgcaagtatcaattttttcagaagacccacaggaggtggcaagattgattgatccctttgtgaatttagtgtctagagtcaacatcaactgccaagcacaaagagggccggatgtgtttccattcactggaaggaaagacagtgcggaaggtaccttgtctgtgttcgacgctttgagaagtttctctatccgtagtcttgttgcagcaaaactgactgattctaacaaagaacttttaaatacaatcgtaagagaacacgacagtaactttctaagcactgattacatcttctaacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgtcaactgctcctgcacaatacactggctacgctgccctgaccgaggagaaaggaaagaagcttgagcttgagaagtggtcatacactccaaagcagtggactgaagatgacgtggatattaaagttgattgctgcggcatgtgtggttcctgtatccatactcttaccaacggttggcctcaacctacatcttacccatgtatcgctgggcatgaggttgtggggacaattgtaagagctgggtcaaggactagccacaaggttggaactagggtcggtgttggtgctcaaggtggaagctgcttagagtgcgactattgcaagcagggcttgcagcagcattgcgagaaaggattgataatgacgtatcagggtacctgggaagacgggtccgtcgcacagggtggttacgctgattttatgagatgccgtggcgctttagctgtacctatacctgacggcttggagtctgagactaccgcaccgttgctgtgcgctggcgttaccgtctacgcgcctttaaagaggtttaacgccggtcccggtaagagagtgggtgtgatcgggattggcggtctgggacaccttgcgctgcaaattgcaaaagcaatgggagctgaggtatatgctttgtctcacagcgcgagcaaaatggacgacgccgagaagcttggatgccctagagagaactttatagtggctaaagaccaagacgaggtcaggaagaagtacaacaagcactttgacatattgatactaacatcttcttccaatgatatacccctagagggattgtacctgccattattacgtaacatgggcactttcgtgatctgcggattacctgaagagaagttgccgtctatgtatggacagatgttggtctacaaatcgataaacttggctggatcattgatcggcgggagttcagaaattgctgagctattcgacttagcacttaagcatgacatcaaggcctgggttgagacaagaccaatgtctgaagttacacaaaccgctcacgatatgcatgcgggaaagtcgcgttaccgttacgttctgaagaattaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgc

SEQ ID NO: 104 - DNA, 6224 bp, synthetic construct
  source 1..6224: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatgtcatccgaaaataccgcaagttttcaccatatcttcaaatcagagaacgaaatccctgaagaatataaagttccagttatacaccagcgtacgtatctgttgaacggggaattagtggaatggaacggggacgtgactgaaatctattcaccagtttgtattccaacagaaaatggcttggaaaggaaattattgggaagtattccaaatataggacctaatgaagctatggaagttcttgaggcttgcgtaaaagcatacgataatgggttgggagaatggccgactatgtcagtggaagggagaattaagtgtatgcaaaagttcgtttatctgatgatacaacaaagagatttaattatcaaactattgatgtgggagattggtaagactttggctgattctacaaaagagtttgataggacggtcgattatattaatcaaacaattgacgccttgaaggatctagatagagaatcttctcgttttcaacaggccgaaggtaccatcgcccaaattaggagagcgcctttgggagtcgtgctgagtatgggtcccttcaattatccgttgaatgaaatcttcacaactttaatccctgcattaataatgggtaatacaattttattcaagttacctaagcatggtgttttggcccattacccccttctagaagcatttaaagaagcttttccaaagggaaccgtaaacacgttgtacggtaagggcagtgaaattataactccaattatggagtccgggaaagtaaacgtgcttgcctttattggttccagcaaggtagctaatggactaaaaaaactacatccaaaggtgaataggctgagagcaattctgagcctagatgcaaagaatgcagcaattgtcacaaagaacgctaatttggacgtggcagtttcagaatgtatattaggagctcttagtttcaatggccagcgttgtactgcattaaaacttatctttgtccagaaggaggttgcactggaatttaccgaaaagttatcagcggctgtgtcggcattgaagcctggattaccttgggaaaaagatgttaaagtaacacctctaccagaagttaataagcctccatatttgaaagaatgcatagatgatgctttgcagaagggagccgcagttttaaataaagatggtgggtataccgatgaatcttttgtatttcccgccgttgtataccccgttaattctgatatgaagctgtatcacgaagaacaatttggtccagtaataccggtagtgccctttgaagatatcgaggagcccattgagtatcaagtcaatgcctctcatggtatgcaagtatcaattttttcagaagacccacaggaggtggcaagattgattgatccctttgtgaatttagtgtctagagtcaacatcaactgccaagcacaaagagggccggatgtgtttccattcactggaaggaaagacagtgcggaaggtaccttgtctgtgttcgacgctttgagaagtttctctatccgtagtcttgttgcagcaaaactgactgattctaacaaagaacttttaaatacaatcgtaagagaacacgacagtaactttctaagcactgattacatcttctaacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgacgctatctaccaagccgaaagtcttgaagctaggtgacttcgagttcagtcaagcaaggtgggatgaactgtcccagattgccgaggtcatcaactgcgaatctcagaatagggatgaattcatcaacgatttgcaaaccaaatacaacgatataacctatattgccagggaatataacagcgttaatgttactggaaggttcgacgcagagatagcacaacatatccccagcactgttaaggggatctgccattgcaccgctggttacgactctatcgatgttgagcccttcagcaagttggggattcagattagtaatgttacggatccagtggtaggacctactgctgacgttgcagtattccttgtgttagcttgtcttaggaatttctttgcaggtcataaaatgttggtatccgggaaatggcctgatgagaaggttcttgagtgtggcggagttgacatgggtaacactcctcaacataaggtactaggaatcttaggtatgggaaacattgggcgtgccgtgaagaataggttgaaagtattcggattcaagaagatcgtatactataatagaaggcaactgtcgccagagctggaagagggcgccgagtacgtaagtcaggacgagttgttgcagaaatccgatgttattgtcgtcgttgttcccttaaatccaaacacgaagcatctaatagaccgtaacgcgatatctaagatgaaggacggagtaatattggtgaacatttctcgtggattggtcttggatgaacacgttgtcacggagttagtgaaatccggcaagataggtgcctttggtgcagacgttttcgagaacgagccgcacgtctcacctgacctttacaacctgcctaacgtggtaacattgccgcacatggggacgcacgcgtttgacgcaactcaggacttgcaggagtgggtgattgagaatattgaatgctgtataaagactggaaaactgaaaactctggtcgcggaacagaaggagctgttctaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgc

SEQ ID NO: 105 - DNA, 6221 bp, synthetic construct
  source 1..6221: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatgaccacggctcaaatattgggtaatcttttccctagtgccggtgatatccctgaaaaatatagattggacggtcagattgaacaaagagaatatttggttgatggagagctaaaaacttggccaggtgccttagcccaagtaagaagccctgtttacttggccggtgctaacggagatgaacaagtaatcttaggctccacgcctctgttggacgccgataccgcgcttaccgctttagatgctgcagtcagggcttacgatcgtggtcaaggtttgtggcctactatgagggtggctgaacgtatacaacatgtagaaacgtttttaggccacatgagagaacaaagggaagcggtggtaaaattgttaatgtgggaaattggaaagaacttgaaagattccgaaaaggaatttgatagaacatgcgattatatagttgatactgtgaacgcattaaaggaattagatagaagatcatcaaggttcgaattagaacaggatacactgggtcaaatccgtagggttccaatgggtgtagctctatgtatgggtccatacaactaccctctgaacgaaacattcactaccttaattcctgccctaattatgggtaatacagtggtttttaagccagctaagcttggagttttgttgattagaccgttgttggaagcatttcgtgattccttccccgctggagtgatcaatgttatctacggttccggtagggaaactgtatctgctctgatggcaagtggtaaaatcgatatattcgcttttataggaactaacaaagcagcctccgatttaaaaaaattgcaccctagaccacatagattgagggcagcactaggtctggacgctaaaaacccaggaatcattttacccgaagttgatttagataatgcagttaatgaggctgtcactggttctctttcattcaacggacagagatgcactgctttgaagattctttttgtacatgaggatgtggtcgaggcctttatcgacaaatttaacgcaaaattggcaacccttaaaccaggcatgccatgggactccggtgttgcattaactccgttgcctgaagctagtaaagtggactatctacattcacttgtagccgacgctgtttcgaaaggagccgccgttgtaaacccgaacggtggtgaagcgcgtgcttcattcttttatccagctgtcctgtaccctgtgactcctcaaatgagagtctatcaagaagagcagtttgggcctgtagttccaatcgttccgtatagacacttagacacggttatagattacgtgttggaatcagattttggccaacaattgagcatcttcggaactaatccagttgcggtcggtaggctggttgacacatttgcaaatcaggtaggaagaataaatctaaatgcacagtgtcagagggggccggatacgtatcccttcaacggccgtaaaaattccgccgagggaaccttgagtgtgcacgacgcgttaagagtcttttccataagaaccttagtagccactaagtttcaagaaactaataaagaactgatctctgatatcatctccggtagatcctcaagctttcttacgacagattacattttttaacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgacgctatctaccaagccgaaagtcttgaagctaggtgacttcgagttcagtcaagcaaggtgggatgaactgtcccagattgccgaggtcatcaactgcgaatctcagaatagggatgaattcatcaacgatttgcaaaccaaatacaacgatataacctatattgccagggaatataacagcgttaatgttactggaaggttcgacgcagagatagcacaacatatccccagcactgttaaggggatctgccattgcaccgctggttacgactctatcgatgttgagcccttcagcaagttggggattcagattagtaatgttacggatccagtggtaggacctactgctgacgttgcagtattccttgtgttagcttgtcttaggaatttctttgcaggtcataaaatgttggtatccgggaaatggcctgatgagaaggttcttgagtgtggcggagttgacatgggtaacactcctcaacataaggtactaggaatcttaggtatgggaaacattgggcgtgccgtgaagaataggttgaaagtattcggattcaagaagatcgtatactataatagaaggcaactgtcgccagagctggaagagggcgccgagtacgtaagtcaggacgagttgttgcagaaatccgatgttattgtcgtcgttgttcccttaaatccaaacacgaagcatctaatagaccgtaacgcgatatctaagatgaaggacggagtaatattggtgaacatttctcgtggattggtcttggatgaacacgttgtcacggagttagtgaaatccggcaagataggtgcctttggtgcagacgttttcgagaacgagccgcacgtctcacctgacctttacaacctgcctaacgtggtaacattgccgcacatggggacgcacgcgtttgacgcaactcaggacttgcaggagtgggtgattgagaatattgaatgctgtataaagactggaaaactgaaaactctggtcgcggaacagaaggagctgttctaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgc

SEQ ID NO: 106 - DNA, 6071 bp, synthetic construct
  source 1..6071: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatggccaagcaatataagaacttggttaatggtgaatggaagttgtctgaaaacgagatcaccatttatgcacctgctactggtgaagaacttggcagcgtccccgccatgactcaagccgaagtagatgccgtttatgcatccgccaagaaggccttatcagactggagagctctgtcatatgttgaaagggcagcttatttgcataaggctgcagatattttggttcgtgacgcagagaagattggtgcaatcttatcaaaagaagtagcaaaaggtcataaagctgcagtttctgaagtgattagaacagctgaaataattaattacgccgcagaagaaggtcttaggatggaaggagaagttcttgaaggcggaagcttcgaagccgcatctaaaaaaaagatcgctattgtcagaagagaaccagtaggtctagtgctagcaatatctccttttaactaccctgtaaatttagcaggcagcaagatagcaccagctttaatagcaggcaatgtagtggctttgaaaccaccaacacagggctcaataagcggtctgctgttagccgaggcttttgctgaagctggtattcctgcaggtgtatttaatacaataactgggagaggttctgttattggtgactatattgttgagcacgaggctgtaaatttcataaattttactggttctactcctattggcgagggtatcggaaagctagcaggtatgaggcctatcatgttagagctgggtggaaaagattctgccatcgtattggaagacgctgatcttgctttggctgcaaaaaacattgtagctggagcatttggttacagtggtcaaagatgcaccgccgtgaagagagttctggttatggataaagtagcagaccaattagctgctgaaatcaagacattggttgaaaagttgtcagtgggtatgccagaagacgacgctgatattactccgcttattgatacaagtgccgccgatttcgtagaaggtcttattaaagacgccacggacaaaggtgcaacagcattaacagcgtttaatagagaaggaaatttaatctcgcctgtgttattcgatcatgtaactacagatatgagattggcctgggaggaaccttttgggcctgtcttaccaattattcgtgttactaccgttgaagaggcaataaaaatttctaatgaaagtgagtacgggttgcaagcttcaattttcactactaattttcctaaagcctttggcattgcagaacaattggaagtaggcactgtacacctgaataacaaaacccaaagaggcaccgataatttcccatttcttggtgctaaaaagtcaggtgctggagttcaaggtgtaaaatattccattgaagctatgacaacagtgaagagtgttgttttcgacatacaataacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgtcaactgctcctgcacaatacactggctacgctgccctgaccgaggagaaaggaaagaagcttgagcttgagaagtggtcatacactccaaagcagtggactgaagatgacgtggatattaaagttgattgctgcggcatgtgtggttcctgtatccatactcttaccaacggttggcctcaacctacatcttacccatgtatcgctgggcatgaggttgtggggacaattgtaagagctgggtcaaggactagccacaaggttggaactagggtcggtgttggtgctcaaggtggaagctgcttagagtgcgactattgcaagcagggcttgcagcagcattgcgagaaaggattgataatgacgtatcagggtacctgggaagacgggtccgtcgcacagggtggttacgctgattttatgagatgccgtggcgctttagctgtacctatacctgacggcttggagtctgagactaccgcaccgttgctgtgcgctggcgttaccgtctacgcgcctttaaagaggtttaacgccggtcccggtaagagagtgggtgtgatcgggattggcggtctgggacaccttgcgctgcaaattgcaaaagcaatgggagctgaggtatatgctttgtctcacagcgcgagcaaaatggacgacgccgagaagcttggatgccctagagagaactttatagtggctaaagaccaagacgaggtcaggaagaagtacaacaagcactttgacatattgatactaacatcttcttccaatgatatacccctagagggattgtacctgccattattacgtaacatgggcactttcgtgatctgcggattacctgaagagaagttgccgtctatgtatggacagatgttggtctacaaatcgataaacttggctggatcattgatcggcgggagttcagaaattgctgagctattcgacttagcacttaagcatgacatcaaggcctgggttgagacaagaccaatgtctgaagttacacaaaccgctcacgatatgcatgcgggaaagtcgcgttaccgttacgttctgaagaattaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgc

SEQ ID NO: 107 - DNA, 6134 bp, synthetic construct
  source 1..6134: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatggcgggcacaggactattcgctgaaatattggatggtgaagtgtacaagtactatgcagatggagagtggaagaccagtagtagtggtaagtcggttgcaataatgaatcctgctacaagaaaaacccaatataaggttcaagcatgcacgcaagaagaagttaatgctgtgatggaattggccaagagcgctcaaaaatcatgggccaaaacaccattgtggaagcgtgctgagttactacataaagccgccgccatattaaaagacaataaggctcctatggcggagtctttagtcaaagagattgctaaacctgcgaaagacagtgttaccgaagttgtaaggtcaggagatctaatatcatattgcgctgaagagggagttcgtatattgggagaaggtaaattcttgctatctgattcatttccaggtaatgatagaacaaaatactgtctgacatcaaagatccctcttggagtagtgttagctatacccccattcaactatccagttaacctggctgtctcaaagatcgcaccagcgttaattgctggtaactcattggttttgaaacccccgacacagggtgcggtgtcttgtcttcacatggttcattgttttcatcttgcaggattcccaaagggtcttatttcttgcataaccggcaaaggcagtgaaataggagattttctaactatgcatcctgcagtaaactgtatttcatttacaggtggcgacactggaatatccataagtaaaaaagctggcatgatcccattacaaatggaattgggtgggaaggatgcatgtattgtattagatgatgctgatttggacttggtagcttcaaatatcattaaaggtggattttcctacagtggtcaaaggtgcacggcggttaaggttgttttggttatggagtcagtggctgatgaactagtcgaaaaggtcaaagcaaaagttgctaaattgacagtgggtccaccagaggagaactctgatattacggcggtagtctcggagagctccgctaattttattgaaggtttagtgatggatgcaaaggaaaaaggtgccacattctgtcaggaatacaagagagaaggaaacctaatctggcctttattgcttgacaatgttagacctgacatgagaatagcatgggaagaacccttcggacctgtcgtccctgttttgagaattaattcagttgaagaaggcattaaccactgtaacgcatctaacttcggtttgcaaggttgtgtgtttactaaagatattaataaggctattcttatttcggatgctatggagacaggcactgtacaaataaacagcgcaccagcaagaggtcctgatcacttccctttccaagggttgaaggattcaggcattggatcgcaaggggttaccaacagcataaatctaatgacgaaagtaaaaactacagttattaacctgcccactccaagctactctatgggctaacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgtcaactgctcctgcacaatacactggctacgctgccctgaccgaggagaaaggaaagaagcttgagcttgagaagtggtcatacactccaaagcagtggactgaagatgacgtggatattaaagttgattgctgcggcatgtgtggttcctgtatccatactcttaccaacggttggcctcaacctacatcttacccatgtatcgctgggcatgaggttgtggggacaattgtaagagctgggtcaaggactagccacaaggttggaactagggtcggtgttggtgctcaaggtggaagctgcttagagtgcgactattgcaagcagggcttgcagcagcattgcgagaaaggattgataatgacgtatcagggtacctgggaagacgggtccgtcgcacagggtggttacgctgattttatgagatgccgtggcgctttagctgtacctatacctgacggcttggagtctgagactaccgcaccgttgctgtgcgctggcgttaccgtctacgcgcctttaaagaggtttaacgccggtcccggtaagagagtgggtgtgatcgggattggcggtctgggacaccttgcgctgcaaattgcaaaagcaatgggagctgaggtatatgctttgtctcacagcgcgagcaaaatggacgacgccgagaagcttggatgccctagagagaactttatagtggctaaagaccaagacgaggtcaggaagaagtacaacaagcactttgacatattgatactaacatcttcttccaatgatatacccctagagggattgtacctgccattattacgtaacatgggcactttcgtgatctgcggattacctgaagagaagttgccgtctatgtatggacagatgttggtctacaaatcgataaacttggctggatcattgatcggcgggagttcagaaattgctgagctattcgacttagcacttaagcatgacatcaaggcctgggttgagacaagaccaatgtctgaagttacacaaaccgctcacgatatgcatgcgggaaagtcgcgttaccgttacgttctgaagaattaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgc

SEQ ID NO: 108 - DNA, 6086 bp, synthetic construct
  source 1..6086: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatggcgggcacaggactattcgctgaaatattggatggtgaagtgtacaagtactatgcagatggagagtggaagaccagtagtagtggtaagtcggttgcaataatgaatcctgctacaagaaaaacccaatataaggttcaagcatgcacgcaagaagaagttaatgctgtgatggaattggccaagagcgctcaaaaatcatgggccaaaacaccattgtggaagcgtgctgagttactacataaagccgccgccatattaaaagacaataaggctcctatggcggagtctttagtcaaagagattgctaaacctgcgaaagacagtgttaccgaagttgtaaggtcaggagatctaatatcatattgcgctgaagagggagttcgtatattgggagaaggtaaattcttgctatctgattcatttccaggtaatgatagaacaaaatactgtctgacatcaaagatccctcttggagtagtgttagctatacccccattcaactatccagttaacctggctgtctcaaagatcgcaccagcgttaattgctggtaactcattggttttgaaacccccgacacagggtgcggtgtcttgtcttcacatggttcattgttttcatcttgcaggattcccaaagggtcttatttcttgcataaccggcaaaggcagtgaaataggagattttctaactatgcatcctgcagtaaactgtatttcatttacaggtggcgacactggaatatccataagtaaaaaagctggcatgatcccattacaaatggaattgggtgggaaggatgcatgtattgtattagatgatgctgatttggacttggtagcttcaaatatcattaaaggtggattttcctacagtggtcaaaggtgcacggcggttaaggttgttttggttatggagtcagtggctgatgaactagtcgaaaaggtcaaagcaaaagttgctaaattgacagtgggtccaccagaggagaactctgatattacggcggtagtctcggagagctccgctaattttattgaaggtttagtgatggatgcaaaggaaaaaggtgccacattctgtcaggaatacaagagagaaggaaacctaatctggcctttattgcttgacaatgttagacctgacatgagaatagcatgggaagaacccttcggacctgtcgtccctgttttgagaattaattcagttgaagaaggcattaaccactgtaacgcatctaacttcggtttgcaaggttgtgtgtttactaaagatattaataaggctattcttatttcggatgctatggagacaggcactgtacaaataaacagcgcaccagcaagaggtcctgatcacttccctttccaagggttgaaggattcaggcattggatcgcaaggggttaccaacagcataaatctaatgacgaaagtaaaaactacagttattaacctgcccactccaagctactctatgggctaacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgacgctatctaccaagccgaaagtcttgaagctaggtgacttcgagttcagtcaagcaaggtgggatgaactgtcccagattgccgaggtcatcaactgcgaatctcagaatagggatgaattcatcaacgatttgcaaaccaaatacaacgatataacctatattgccagggaatataacagcgttaatgttactggaaggttcgacgcagagatagcacaacatatccccagcactgttaaggggatctgccattgcaccgctggttacgactctatcgatgttgagcccttcagcaagttggggattcagattagtaatgttacggatccagtggtaggacctactgctgacgttgcagtattccttgtgttagcttgtcttaggaatttctttgcaggtcataaaatgttggtatccgggaaatggcctgatgagaaggttcttgagtgtggcggagttgacatgggtaacactcctcaacataaggtactaggaatcttaggtatgggaaacattgggcgtgccgtgaagaataggttgaaagtattcggattcaagaagatcgtatactataatagaaggcaactgtcgccagagctggaagagggcgccgagtacgtaagtcaggacgagttgttgcagaaatccgatgttattgtcgtcgttgttcccttaaatccaaacacgaagcatctaatagaccgtaacgcgatatctaagatgaaggacggagtaatattggtgaacatttctcgtggattggtcttggatgaacacgttgtcacggagttagtgaaatccggcaagataggtgcctttggtgcagacgttttcgagaacgagccgcacgtctcacctgacctttacaacctgcctaacgtggtaacattgccgcacatggggacgcacgcgtttgacgcaactcaggacttgcaggagtgggtgattgagaatattgaatgctgtataaagactggaaaactgaaaactctggtcgcggaacagaaggagctgttctaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgc

SEQ ID NO: 109 - DNA, 6269 bp, synthetic construct
  source 1..6269: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatgaccacggctcaaatattgggtaatcttttccctagtgccggtgatatccctgaaaaatatagattggacggtcagattgaacaaagagaatatttggttgatggagagctaaaaacttggccaggtgccttagcccaagtaagaagccctgtttacttggccggtgctaacggagatgaacaagtaatcttaggctccacgcctctgttggacgccgataccgcgcttaccgctttagatgctgcagtcagggcttacgatcgtggtcaaggtttgtggcctactatgagggtggctgaacgtatacaacatgtagaaacgtttttaggccacatgagagaacaaagggaagcggtggtaaaattgttaatgtgggaaattggaaagaacttgaaagattccgaaaaggaatttgatagaacatgcgattatatagttgatactgtgaacgcattaaaggaattagatagaagatcatcaaggttcgaattagaacaggatacactgggtcaaatccgtagggttccaatgggtgtagctctatgtatgggtccatacaactaccctctgaacgaaacattcactaccttaattcctgccctaattatgggtaatacagtggtttttaagccagctaagcttggagttttgttgattagaccgttgttggaagcatttcgtgattccttccccgctggagtgatcaatgttatctacggttccggtagggaaactgtatctgctctgatggcaagtggtaaaatcgatatattcgcttttataggaactaacaaagcagcctccgatttaaaaaaattgcaccctagaccacatagattgagggcagcactaggtctggacgctaaaaacccaggaatcattttacccgaagttgatttagataatgcagttaatgaggctgtcactggttctctttcattcaacggacagagatgcactgctttgaagattctttttgtacatgaggatgtggtcgaggcctttatcgacaaatttaacgcaaaattggcaacccttaaaccaggcatgccatgggactccggtgttgcattaactccgttgcctgaagctagtaaagtggactatctacattcacttgtagccgacgctgtttcgaaaggagccgccgttgtaaacccgaacggtggtgaagcgcgtgcttcattcttttatccagctgtcctgtaccctgtgactcctcaaatgagagtctatcaagaagagcagtttgggcctgtagttccaatcgttccgtatagacacttagacacggttatagattacgtgttggaatcagattttggccaacaattgagcatcttcggaactaatccagttgcggtcggtaggctggttgacacatttgcaaatcaggtaggaagaataaatctaaatgcacagtgtcagagggggccggatacgtatcccttcaacggccgtaaaaattccgccgagggaaccttgagtgtgcacgacgcgttaagagtcttttccataagaaccttagtagccactaagtttcaagaaactaataaagaactgatctctgatatcatctccggtagatcctcaagctttcttacgacagattacattttttaacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgtcaactgctcctgcacaatacactggctacgctgccctgaccgaggagaaaggaaagaagcttgagcttgagaagtggtcatacactccaaagcagtggactgaagatgacgtggatattaaagttgattgctgcggcatgtgtggttcctgtatccatactcttaccaacggttggcctcaacctacatcttacccatgtatcgctgggcatgaggttgtggggacaattgtaagagctgggtcaaggactagccacaaggttggaactagggtcggtgttggtgctcaaggtggaagctgcttagagtgcgactattgcaagcagggcttgcagcagcattgcgagaaaggattgataatgacgtatcagggtacctgggaagacgggtccgtcgcacagggtggttacgctgattttatgagatgccgtggcgctttagctgtacctatacctgacggcttggagtctgagactaccgcaccgttgctgtgcgctggcgttaccgtctacgcgcctttaaagaggtttaacgccggtcccggtaagagagtgggtgtgatcgggattggcggtctgggacaccttgcgctgcaaattgcaaaagcaatgggagctgaggtatatgctttgtctcacagcgcgagcaaaatggacgacgccgagaagcttggatgccctagagagaactttatagtggctaaagaccaagacgaggtcaggaagaagtacaacaagcactttgacatattgatactaacatcttcttccaatgatatacccctagagggattgtacctgccattattacgtaacatgggcactttcgtgatctgcggattacctgaagagaagttgccgtctatgtatggacagatgttggtctacaaatcgataaacttggctggatcattgatcggcgggagttcagaaattgctgagctattcgacttagcacttaagcatgacatcaaggcctgggttgagacaagaccaatgtctgaagttacacaaaccgctcacgatatgcatgcgggaaagtcgcgttaccgttacgttctgaagaattaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatcatgtcgaaagctacatataaggaacgtgctgctactcatcctagtcctgttgctgccaagctatttaatatcatgcacgaaaagcaaacaaacttgtgtgcttcattggatgttcgtaccaccaaggaattactggagttagttgaagcattaggtcccaaaatttgtttactaaaaacacatgtggatatcttgactgatttttccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacaccaacgc

SEQ ID NO: 110 - DNA, 1127 bp, synthetic construct
  source 1..1127: mol_type = other DNA
gctccatggagggcacagttaagccgctaaaggcattatccgccaagtacaattttttactcttcgaagacagaaaatttgctgacattggtaatacagtcaaattgcagtactctgcgggtgtatacagaatagcagaatgggcagacattacgaatgcacacggtgtggtgggcccaggtattgttagcggtttgaagcaggcggcggaagaagtaacaaaggaacctagaggccttttgatgttagcagaattgtcatgcaagggctccctagctactggagaatatactaagggtactgttgacattgcgaagagcgacaaagattttgttatcggctttattgctcaaagagacatgggtggaagagatgaaggttacgattggttgattatgacacccggtgtgggtttagatgacaagggagacgcattgggtcaacagtatagaaccgtggatgatgtggtctctacaggatctgacattattattgttggaagaggactatttgcaaagggaagggatgctaaggtagagggtgaacgttacagaaaagcaggctgggaagcatatttgagaagatgcggccagcaaaactaaaaaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttgataacttcgtataatgtatgctatacgaagttatctcgaggataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtc

SEQ ID NO: 111 - DNA, 6406 bp, synthetic construct
  source 1..6406: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatggccaagcaatataagaacttggttaatggtgaatggaagttgtctgaaaacgagatcaccatttatgcacctgctactggtgaagaacttggcagcgtccccgccatgactcaagccgaagtagatgccgtttatgcatccgccaagaaggccttatcagactggagagctctgtcatatgttgaaagggcagcttatttgcataaggctgcagatattttggttcgtgacgcagagaagattggtgcaatcttatcaaaagaagtagcaaaaggtcataaagctgcagtttctgaagtgattagaacagctgaaataattaattacgccgcagaagaaggtcttaggatggaaggagaagttcttgaaggcggaagcttcgaagccgcatctaaaaaaaagatcgctattgtcagaagagaaccagtaggtctagtgctagcaatatctccttttaactaccctgtaaatttagcaggcagcaagatagcaccagctttaatagcaggcaatgtagtggctttgaaaccaccaacacagggctcaataagcggtctgctgttagccgaggcttttgctgaagctggtattcctgcaggtgtatttaatacaataactgggagaggttctgttattggtgactatattgttgagcacgaggctgtaaatttcataaattttactggttctactcctattggcgagggtatcggaaagctagcaggtatgaggcctatcatgttagagctgggtggaaaagattctgccatcgtattggaagacgctgatcttgctttggctgcaaaaaacattgtagctggagcatttggttacagtggtcaaagatgcaccgccgtgaagagagttctggttatggataaagtagcagaccaattagctgctgaaatcaagacattggttgaaaagttgtcagtgggtatgccagaagacgacgctgatattactccgcttattgatacaagtgccgccgatttcgtagaaggtcttattaaagacgccacggacaaaggtgcaacagcattaacagcgtttaatagagaaggaaatttaatctcgcctgtgttattcgatcatgtaactacagatatgagattggcctgggaggaaccttttgggcctgtcttaccaattattcgtgttactaccgttgaagaggcaataaaaatttctaatgaaagtgagtacgggttgcaagcttcaattttcactactaattttcctaaagcctttggcattgcagaacaattggaagtaggcactgtacacctgaataacaaaacccaaagaggcaccgataatttcccatttcttggtgctaaaaagtcaggtgctggagttcaaggtgtaaaatattccattgaagctatgacaacagtgaagagtgttgttttcgacatacaataacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgacgctatctaccaagccgaaagtcttgaagctaggtgacttcgagttcagtcaagcaaggtgggatgaactgtcccagattgccgaggtcatcaactgcgaatctcagaatagggatgaattcatcaacgatttgcaaaccaaatacaacgatataacctatattgccagggaatataacagcgttaatgttactggaaggttcgacgcagagatagcacaacatatccccagcactgttaaggggatctgccattgcaccgctggttacgactctatcgatgttgagcccttcagcaagttggggattcagattagtaatgttacggatccagtggtaggacctactgctgacgttgcagtattccttgtgttagcttgtcttaggaatttctttgcaggtcataaaatgttggtatccgggaaatggcctgatgagaaggttcttgagtgtggcggagttgacatgggtaacactcctcaacataaggtactaggaatcttaggtatgggaaacattgggcgtgccgtgaagaataggttgaaagtattcggattcaagaagatcgtatactataatagaaggcaactgtcgccagagctggaagagggcgccgagtacgtaagtcaggacgagttgttgcagaaatccgatgttattgtcgtcgttgttcccttaaatccaaacacgaagcatctaatagaccgtaacgcgatatctaagatgaaggacggagtaatattggtgaacatttctcgtggattggtcttggatgaacacgttgtcacggagttagtgaaatccggcaagataggtgcctttggtgcagacgttttcgagaacgagccgcacgtctcacctgacctttacaacctgcctaacgtggtaacattgccgcacatggggacgcacgcgtttgacgcaactcaggacttgcaggagtgggtgattgagaatattgaatgctgtataaagactggaaaactgaaaactctggtcgcggaacagaaggagctgttctaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagagcgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtttccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataaaagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgcgtccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttgcaacgc

SEQ ID NO: 112 - DNA, 6655 bp, synthetic construct
  source 1..6655: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatgtcatccgaaaataccgcaagttttcaccatatcttcaaatcagagaacgaaatccctgaagaatataaagttccagttatacaccagcgtacgtatctgttgaacggggaattagtggaatggaacggggacgtgactgaaatctattcaccagtttgtattccaacagaaaatggcttggaaaggaaattattgggaagtattccaaatataggacctaatgaagctatggaagttcttgaggcttgcgtaaaagcatacgataatgggttgggagaatggccgactatgtcagtggaagggagaattaagtgtatgcaaaagttcgtttatctgatgatacaacaaagagatttaattatcaaactattgatgtgggagattggtaagactttggctgattctacaaaagagtttgataggacggtcgattatattaatcaaacaattgacgccttgaaggatctagatagagaatcttctcgttttcaacaggccgaaggtaccatcgcccaaattaggagagcgcctttgggagtcgtgctgagtatgggtcccttcaattatccgttgaatgaaatcttcacaactttaatccctgcattaataatgggtaatacaattttattcaagttacctaagcatggtgttttggcccattacccccttctagaagcatttaaagaagcttttccaaagggaaccgtaaacacgttgtacggtaagggcagtgaaattataactccaattatggagtccgggaaagtaaacgtgcttgcctttattggttccagcaaggtagctaatggactaaaaaaactacatccaaaggtgaataggctgagagcaattctgagcctagatgcaaagaatgcagcaattgtcacaaagaacgctaatttggacgtggcagtttcagaatgtatattaggagctcttagtttcaatggccagcgttgtactgcattaaaacttatctttgtccagaaggaggttgcactggaatttaccgaaaagttatcagcggctgtgtcggcattgaagcctggattaccttgggaaaaagatgttaaagtaacacctctaccagaagttaataagcctccatatttgaaagaatgcatagatgatgctttgcagaagggagccgcagttttaaataaagatggtgggtataccgatgaatcttttgtatttcccgccgttgtataccccgttaattctgatatgaagctgtatcacgaagaacaatttggtccagtaataccggtagtgccctttgaagatatcgaggagcccattgagtatcaagtcaatgcctctcatggtatgcaagtatcaattttttcagaagacccacaggaggtggcaagattgattgatccctttgtgaatttagtgtctagagtcaacatcaactgccaagcacaaagagggccggatgtgtttccattcactggaaggaaagacagtgcggaaggtaccttgtctgtgttcgacgctttgagaagtttctctatccgtagtcttgttgcagcaaaactgactgattctaacaaagaacttttaaatacaatcgtaagagaacacgacagtaactttctaagcactgattacatcttctaacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgtcaactgctcctgcacaatacactggctacgctgccctgaccgaggagaaaggaaagaagcttgagcttgagaagtggtcatacactccaaagcagtggactgaagatgacgtggatattaaagttgattgctgcggcatgtgtggttcctgtatccatactcttaccaacggttggcctcaacctacatcttacccatgtatcgctgggcatgaggttgtggggacaattgtaagagctgggtcaaggactagccacaaggttggaactagggtcggtgttggtgctcaaggtggaagctgcttagagtgcgactattgcaagcagggcttgcagcagcattgcgagaaaggattgataatgacgtatcagggtacctgggaagacgggtccgtcgcacagggtggttacgctgattttatgagatgccgtggcgctttagctgtacctatacctgacggcttggagtctgagactaccgcaccgttgctgtgcgctggcgttaccgtctacgcgcctttaaagaggtttaacgccggtcccggtaagagagtgggtgtgatcgggattggcggtctgggacaccttgcgctgcaaattgcaaaagcaatgggagctgaggtatatgctttgtctcacagcgcgagcaaaatggacgacgccgagaagcttggatgccctagagagaactttatagtggctaaagaccaagacgaggtcaggaagaagtacaacaagcactttgacatattgatactaacatcttcttccaatgatatacccctagagggattgtacctgccattattacgtaacatgggcactttcgtgatctgcggattacctgaagagaagttgccgtctatgtatggacagatgttggtctacaaatcgataaacttggctggatcattgatcggcgggagttcagaaattgctgagctattcgacttagcacttaagcatgacatcaaggcctgggttgagacaagaccaatgtctgaagttacacaaaccgctcacgatatgcatgcgggaaagtcgcgttaccgttacgttctgaagaattaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagagcgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtttccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataaaagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgcgtccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttgcaacgc

SEQ ID NO: 113 - DNA, 6607 bp, synthetic construct
  source 1..6607: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatgtcatccgaaaataccgcaagttttcaccatatcttcaaatcagagaacgaaatccctgaagaatataaagttccagttatacaccagcgtacgtatctgttgaacggggaattagtggaatggaacggggacgtgactgaaatctattcaccagtttgtattccaacagaaaatggcttggaaaggaaattattgggaagtattccaaatataggacctaatgaagctatggaagttcttgaggcttgcgtaaaagcatacgataatgggttgggagaatggccgactatgtcagtggaagggagaattaagtgtatgcaaaagttcgtttatctgatgatacaacaaagagatttaattatcaaactattgatgtgggagattggtaagactttggctgattctacaaaagagtttgataggacggtcgattatattaatcaaacaattgacgccttgaaggatctagatagagaatcttctcgttttcaacaggccgaaggtaccatcgcccaaattaggagagcgcctttgggagtcgtgctgagtatgggtcccttcaattatccgttgaatgaaatcttcacaactttaatccctgcattaataatgggtaatacaattttattcaagttacctaagcatggtgttttggcccattacccccttctagaagcatttaaagaagcttttccaaagggaaccgtaaacacgttgtacggtaagggcagtgaaattataactccaattatggagtccgggaaagtaaacgtgcttgcctttattggttccagcaaggtagctaatggactaaaaaaactacatccaaaggtgaataggctgagagcaattctgagcctagatgcaaagaatgcagcaattgtcacaaagaacgctaatttggacgtggcagtttcagaatgtatattaggagctcttagtttcaatggccagcgttgtactgcattaaaacttatctttgtccagaaggaggttgcactggaatttaccgaaaagttatcagcggctgtgtcggcattgaagcctggattaccttgggaaaaagatgttaaagtaacacctctaccagaagttaataagcctccatatttgaaagaatgcatagatgatgctttgcagaagggagccgcagttttaaataaagatggtgggtataccgatgaatcttttgtatttcccgccgttgtataccccgttaattctgatatgaagctgtatcacgaagaacaatttggtccagtaataccggtagtgccctttgaagatatcgaggagcccattgagtatcaagtcaatgcctctcatggtatgcaagtatcaattttttcagaagacccacaggaggtggcaagattgattgatccctttgtgaatttagtgtctagagtcaacatcaactgccaagcacaaagagggccggatgtgtttccattcactggaaggaaagacagtgcggaaggtaccttgtctgtgttcgacgctttgagaagtttctctatccgtagtcttgttgcagcaaaactgactgattctaacaaagaacttttaaatacaatcgtaagagaacacgacagtaactttctaagcactgattacatcttctaacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgacgctatctaccaagccgaaagtcttgaagctaggtgacttcgagttcagtcaagcaaggtgggatgaactgtcccagattgccgaggtcatcaactgcgaatctcagaatagggatgaattcatcaacgatttgcaaaccaaatacaacgatataacctatattgccagggaatataacagcgttaatgttactggaaggttcgacgcagagatagcacaacatatccccagcactgttaaggggatctgccattgcaccgctggttacgactctatcgatgttgagcccttcagcaagttggggattcagattagtaatgttacggatccagtggtaggacctactgctgacgttgcagtattccttgtgttagcttgtcttaggaatttctttgcaggtcataaaatgttggtatccgggaaatggcctgatgagaaggttcttgagtgtggcggagttgacatgggtaacactcctcaacataaggtactaggaatcttaggtatgggaaacattgggcgtgccgtgaagaataggttgaaagtattcggattcaagaagatcgtatactataatagaaggcaactgtcgccagagctggaagagggcgccgagtacgtaagtcaggacgagttgttgcagaaatccgatgttattgtcgtcgttgttcccttaaatccaaacacgaagcatctaatagaccgtaacgcgatatctaagatgaaggacggagtaatattggtgaacatttctcgtggattggtcttggatgaacacgttgtcacggagttagtgaaatccggcaagataggtgcctttggtgcagacgttttcgagaacgagccgcacgtctcacctgacctttacaacctgcctaacgtggtaacattgccgcacatggggacgcacgcgtttgacgcaactcaggacttgcaggagtgggtgattgagaatattgaatgctgtataaagactggaaaactgaaaactctggtcgcggaacagaaggagctgttctaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagagcgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtttccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataaaagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgcgtccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttgcaacgc

SEQ ID NO: 114 - DNA, 6454 bp, synthetic construct
  source 1..6454: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatggccaagcaatataagaacttggttaatggtgaatggaagttgtctgaaaacgagatcaccatttatgcacctgctactggtgaagaacttggcagcgtccccgccatgactcaagccgaagtagatgccgtttatgcatccgccaagaaggccttatcagactggagagctctgtcatatgttgaaagggcagcttatttgcataaggctgcagatattttggttcgtgacgcagagaagattggtgcaatcttatcaaaagaagtagcaaaaggtcataaagctgcagtttctgaagtgattagaacagctgaaataattaattacgccgcagaagaaggtcttaggatggaaggagaagttcttgaaggcggaagcttcgaagccgcatctaaaaaaaagatcgctattgtcagaagagaaccagtaggtctagtgctagcaatatctccttttaactaccctgtaaatttagcaggcagcaagatagcaccagctttaatagcaggcaatgtagtggctttgaaaccaccaacacagggctcaataagcggtctgctgttagccgaggcttttgctgaagctggtattcctgcaggtgtatttaatacaataactgggagaggttctgttattggtgactatattgttgagcacgaggctgtaaatttcataaattttactggttctactcctattggcgagggtatcggaaagctagcaggtatgaggcctatcatgttagagctgggtggaaaagattctgccatcgtattggaagacgctgatcttgctttggctgcaaaaaacattgtagctggagcatttggttacagtggtcaaagatgcaccgccgtgaagagagttctggttatggataaagtagcagaccaattagctgctgaaatcaagacattggttgaaaagttgtcagtgggtatgccagaagacgacgctgatattactccgcttattgatacaagtgccgccgatttcgtagaaggtcttattaaagacgccacggacaaaggtgcaacagcattaacagcgtttaatagagaaggaaatttaatctcgcctgtgttattcgatcatgtaactacagatatgagattggcctgggaggaaccttttgggcctgtcttaccaattattcgtgttactaccgttgaagaggcaataaaaatttctaatgaaagtgagtacgggttgcaagcttcaattttcactactaattttcctaaagcctttggcattgcagaacaattggaagtaggcactgtacacctgaataacaaaacccaaagaggcaccgataatttcccatttcttggtgctaaaaagtcaggtgctggagttcaaggtgtaaaatattccattgaagctatgacaacagtgaagagtgttgttttcgacatacaataacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgtcaactgctcctgcacaatacactggctacgctgccctgaccgaggagaaaggaaagaagcttgagcttgagaagtggtcatacactccaaagcagtggactgaagatgacgtggatattaaagttgattgctgcggcatgtgtggttcctgtatccatactcttaccaacggttggcctcaacctacatcttacccatgtatcgctgggcatgaggttgtggggacaattgtaagagctgggtcaaggactagccacaaggttggaactagggtcggtgttggtgctcaaggtggaagctgcttagagtgcgactattgcaagcagggcttgcagcagcattgcgagaaaggattgataatgacgtatcagggtacctgggaagacgggtccgtcgcacagggtggttacgctgattttatgagatgccgtggcgctttagctgtacctatacctgacggcttggagtctgagactaccgcaccgttgctgtgcgctggcgttaccgtctacgcgcctttaaagaggtttaacgccggtcccggtaagagagtgggtgtgatcgggattggcggtctgggacaccttgcgctgcaaattgcaaaagcaatgggagctgaggtatatgctttgtctcacagcgcgagcaaaatggacgacgccgagaagcttggatgccctagagagaactttatagtggctaaagaccaagacgaggtcaggaagaagtacaacaagcactttgacatattgatactaacatcttcttccaatgatatacccctagagggattgtacctgccattattacgtaacatgggcactttcgtgatctgcggattacctgaagagaagttgccgtctatgtatggacagatgttggtctacaaatcgataaacttggctggatcattgatcggcgggagttcagaaattgctgagctattcgacttagcacttaagcatgacatcaaggcctgggttgagacaagaccaatgtctgaagttacacaaaccgctcacgatatgcatgcgggaaagtcgcgttaccgttacgttctgaagaattaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagagcgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtttccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataaaagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgcgtccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttgcaacgc

SEQ ID NO: 115 - DNA, 6517 bp, synthetic construct
  source 1..6517: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatggcgggcacaggactattcgctgaaatattggatggtgaagtgtacaagtactatgcagatggagagtggaagaccagtagtagtggtaagtcggttgcaataatgaatcctgctacaagaaaaacccaatataaggttcaagcatgcacgcaagaagaagttaatgctgtgatggaattggccaagagcgctcaaaaatcatgggccaaaacaccattgtggaagcgtgctgagttactacataaagccgccgccatattaaaagacaataaggctcctatggcggagtctttagtcaaagagattgctaaacctgcgaaagacagtgttaccgaagttgtaaggtcaggagatctaatatcatattgcgctgaagagggagttcgtatattgggagaaggtaaattcttgctatctgattcatttccaggtaatgatagaacaaaatactgtctgacatcaaagatccctcttggagtagtgttagctatacccccattcaactatccagttaacctggctgtctcaaagatcgcaccagcgttaattgctggtaactcattggttttgaaacccccgacacagggtgcggtgtcttgtcttcacatggttcattgttttcatcttgcaggattcccaaagggtcttatttcttgcataaccggcaaaggcagtgaaataggagattttctaactatgcatcctgcagtaaactgtatttcatttacaggtggcgacactggaatatccataagtaaaaaagctggcatgatcccattacaaatggaattgggtgggaaggatgcatgtattgtattagatgatgctgatttggacttggtagcttcaaatatcattaaaggtggattttcctacagtggtcaaaggtgcacggcggttaaggttgttttggttatggagtcagtggctgatgaactagtcgaaaaggtcaaagcaaaagttgctaaattgacagtgggtccaccagaggagaactctgatattacggcggtagtctcggagagctccgctaattttattgaaggtttagtgatggatgcaaaggaaaaaggtgccacattctgtcaggaatacaagagagaaggaaacctaatctggcctttattgcttgacaatgttagacctgacatgagaatagcatgggaagaacccttcggacctgtcgtccctgttttgagaattaattcagttgaagaaggcattaaccactgtaacgcatctaacttcggtttgcaaggttgtgtgtttactaaagatattaataaggctattcttatttcggatgctatggagacaggcactgtacaaataaacagcgcaccagcaagaggtcctgatcacttccctttccaagggttgaaggattcaggcattggatcgcaaggggttaccaacagcataaatctaatgacgaaagtaaaaactacagttattaacctgcccactccaagctactctatgggctaacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgtcaactgctcctgcacaatacactggctacgctgccctgaccgaggagaaaggaaagaagcttgagcttgagaagtggtcatacactccaaagcagtggactgaagatgacgtggatattaaagttgattgctgcggcatgtgtggttcctgtatccatactcttaccaacggttggcctcaacctacatcttacccatgtatcgctgggcatgaggttgtggggacaattgtaagagctgggtcaaggactagccacaaggttggaactagggtcggtgttggtgctcaaggtggaagctgcttagagtgcgactattgcaagcagggcttgcagcagcattgcgagaaaggattgataatgacgtatcagggtacctgggaagacgggtccgtcgcacagggtggttacgctgattttatgagatgccgtggcgctttagctgtacctatacctgacggcttggagtctgagactaccgcaccgttgctgtgcgctggcgttaccgtctacgcgcctttaaagaggtttaacgccggtcccggtaagagagtgggtgtgatcgggattggcggtctgggacaccttgcgctgcaaattgcaaaagcaatgggagctgaggtatatgctttgtctcacagcgcgagcaaaatggacgacgccgagaagcttggatgccctagagagaactttatagtggctaaagaccaagacgaggtcaggaagaagtacaacaagcactttgacatattgatactaacatcttcttccaatgatatacccctagagggattgtacctgccattattacgtaacatgggcactttcgtgatctgcggattacctgaagagaagttgccgtctatgtatggacagatgttggtctacaaatcgataaacttggctggatcattgatcggcgggagttcagaaattgctgagctattcgacttagcacttaagcatgacatcaaggcctgggttgagacaagaccaatgtctgaagttacacaaaccgctcacgatatgcatgcgggaaagtcgcgttaccgttacgttctgaagaattaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagagcgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtttccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataaaagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgcgtccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttgcaacgc

SEQ ID NO: 116 - DNA, 6469 bp, synthetic construct
  source 1..6469: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatggcgggcacaggactattcgctgaaatattggatggtgaagtgtacaagtactatgcagatggagagtggaagaccagtagtagtggtaagtcggttgcaataatgaatcctgctacaagaaaaacccaatataaggttcaagcatgcacgcaagaagaagttaatgctgtgatggaattggccaagagcgctcaaaaatcatgggccaaaacaccattgtggaagcgtgctgagttactacataaagccgccgccatattaaaagacaataaggctcctatggcggagtctttagtcaaagagattgctaaacctgcgaaagacagtgttaccgaagttgtaaggtcaggagatctaatatcatattgcgctgaagagggagttcgtatattgggagaaggtaaattcttgctatctgattcatttccaggtaatgatagaacaaaatactgtctgacatcaaagatccctcttggagtagtgttagctatacccccattcaactatccagttaacctggctgtctcaaagatcgcaccagcgttaattgctggtaactcattggttttgaaacccccgacacagggtgcggtgtcttgtcttcacatggttcattgttttcatcttgcaggattcccaaagggtcttatttcttgcataaccggcaaaggcagtgaaataggagattttctaactatgcatcctgcagtaaactgtatttcatttacaggtggcgacactggaatatccataagtaaaaaagctggcatgatcccattacaaatggaattgggtgggaaggatgcatgtattgtattagatgatgctgatttggacttggtagcttcaaatatcattaaaggtggattttcctacagtggtcaaaggtgcacggcggttaaggttgttttggttatggagtcagtggctgatgaactagtcgaaaaggtcaaagcaaaagttgctaaattgacagtgggtccaccagaggagaactctgatattacggcggtagtctcggagagctccgctaattttattgaaggtttagtgatggatgcaaaggaaaaaggtgccacattctgtcaggaatacaagagagaaggaaacctaatctggcctttattgcttgacaatgttagacctgacatgagaatagcatgggaagaacccttcggacctgtcgtccctgttttgagaattaattcagttgaagaaggcattaaccactgtaacgcatctaacttcggtttgcaaggttgtgtgtttactaaagatattaataaggctattcttatttcggatgctatggagacaggcactgtacaaataaacagcgcaccagcaagaggtcctgatcacttccctttccaagggttgaaggattcaggcattggatcgcaaggggttaccaacagcataaatctaatgacgaaagtaaaaactacagttattaacctgcccactccaagctactctatgggctaacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgacgctatctaccaagccgaaagtcttgaagctaggtgacttcgagttcagtcaagcaaggtgggatgaactgtcccagattgccgaggtcatcaactgcgaatctcagaatagggatgaattcatcaacgatttgcaaaccaaatacaacgatataacctatattgccagggaatataacagcgttaatgttactggaaggttcgacgcagagatagcacaacatatccccagcactgttaaggggatctgccattgcaccgctggttacgactctatcgatgttgagcccttcagcaagttggggattcagattagtaatgttacggatccagtggtaggacctactgctgacgttgcagtattccttgtgttagcttgtcttaggaatttctttgcaggtcataaaatgttggtatccgggaaatggcctgatgagaaggttcttgagtgtggcggagttgacatgggtaacactcctcaacataaggtactaggaatcttaggtatgggaaacattgggcgtgccgtgaagaataggttgaaagtattcggattcaagaagatcgtatactataatagaaggcaactgtcgccagagctggaagagggcgccgagtacgtaagtcaggacgagttgttgcagaaatccgatgttattgtcgtcgttgttcccttaaatccaaacacgaagcatctaatagaccgtaacgcgatatctaagatgaaggacggagtaatattggtgaacatttctcgtggattggtcttggatgaacacgttgtcacggagttagtgaaatccggcaagataggtgcctttggtgcagacgttttcgagaacgagccgcacgtctcacctgacctttacaacctgcctaacgtggtaacattgccgcacatggggacgcacgcgtttgacgcaactcaggacttgcaggagtgggtgattgagaatattgaatgctgtataaagactggaaaactgaaaactctggtcgcggaacagaaggagctgttctaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagagcgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtttccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataaaagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgcgtccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttgcaacgc

SEQ ID NO: 117 - DNA, 6652 bp, synthetic construct
  source 1..6652: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatgaccacggctcaaatattgggtaatcttttccctagtgccggtgatatccctgaaaaatatagattggacggtcagattgaacaaagagaatatttggttgatggagagctaaaaacttggccaggtgccttagcccaagtaagaagccctgtttacttggccggtgctaacggagatgaacaagtaatcttaggctccacgcctctgttggacgccgataccgcgcttaccgctttagatgctgcagtcagggcttacgatcgtggtcaaggtttgtggcctactatgagggtggctgaacgtatacaacatgtagaaacgtttttaggccacatgagagaacaaagggaagcggtggtaaaattgttaatgtgggaaattggaaagaacttgaaagattccgaaaaggaatttgatagaacatgcgattatatagttgatactgtgaacgcattaaaggaattagatagaagatcatcaaggttcgaattagaacaggatacactgggtcaaatccgtagggttccaatgggtgtagctctatgtatgggtccatacaactaccctctgaacgaaacattcactaccttaattcctgccctaattatgggtaatacagtggtttttaagccagctaagcttggagttttgttgattagaccgttgttggaagcatttcgtgattccttccccgctggagtgatcaatgttatctacggttccggtagggaaactgtatctgctctgatggcaagtggtaaaatcgatatattcgcttttataggaactaacaaagcagcctccgatttaaaaaaattgcaccctagaccacatagattgagggcagcactaggtctggacgctaaaaacccaggaatcattttacccgaagttgatttagataatgcagttaatgaggctgtcactggttctctttcattcaacggacagagatgcactgctttgaagattctttttgtacatgaggatgtggtcgaggcctttatcgacaaatttaacgcaaaattggcaacccttaaaccaggcatgccatgggactccggtgttgcattaactccgttgcctgaagctagtaaagtggactatctacattcacttgtagccgacgctgtttcgaaaggagccgccgttgtaaacccgaacggtggtgaagcgcgtgcttcattcttttatccagctgtcctgtaccctgtgactcctcaaatgagagtctatcaagaagagcagtttgggcctgtagttccaatcgttccgtatagacacttagacacggttatagattacgtgttggaatcagattttggccaacaattgagcatcttcggaactaatccagttgcggtcggtaggctggttgacacatttgcaaatcaggtaggaagaataaatctaaatgcacagtgtcagagggggccggatacgtatcccttcaacggccgtaaaaattccgccgagggaaccttgagtgtgcacgacgcgttaagagtcttttccataagaaccttagtagccactaagtttcaagaaactaataaagaactgatctctgatatcatctccggtagatcctcaagctttcttacgacagattacattttttaacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgtcaactgctcctgcacaatacactggctacgctgccctgaccgaggagaaaggaaagaagcttgagcttgagaagtggtcatacactccaaagcagtggactgaagatgacgtggatattaaagttgattgctgcggcatgtgtggttcctgtatccatactcttaccaacggttggcctcaacctacatcttacccatgtatcgctgggcatgaggttgtggggacaattgtaagagctgggtcaaggactagccacaaggttggaactagggtcggtgttggtgctcaaggtggaagctgcttagagtgcgactattgcaagcagggcttgcagcagcattgcgagaaaggattgataatgacgtatcagggtacctgggaagacgggtccgtcgcacagggtggttacgctgattttatgagatgccgtggcgctttagctgtacctatacctgacggcttggagtctgagactaccgcaccgttgctgtgcgctggcgttaccgtctacgcgcctttaaagaggtttaacgccggtcccggtaagagagtgggtgtgatcgggattggcggtctgggacaccttgcgctgcaaattgcaaaagcaatgggagctgaggtatatgctttgtctcacagcgcgagcaaaatggacgacgccgagaagcttggatgccctagagagaactttatagtggctaaagaccaagacgaggtcaggaagaagtacaacaagcactttgacatattgatactaacatcttcttccaatgatatacccctagagggattgtacctgccattattacgtaacatgggcactttcgtgatctgcggattacctgaagagaagttgccgtctatgtatggacagatgttggtctacaaatcgataaacttggctggatcattgatcggcgggagttcagaaattgctgagctattcgacttagcacttaagcatgacatcaaggcctgggttgagacaagaccaatgtctgaagttacacaaaccgctcacgatatgcatgcgggaaagtcgcgttaccgttacgttctgaagaattaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagagcgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtttccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataaaagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgcgtccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttgcaacgc

SEQ ID NO: 118 - DNA, 6604 bp, synthetic construct
  source 1..6604: mol_type = other DNA
ggccgcatcgaaggctttttttgtctgtcttccttgggtcttaccccgccacgcgttaatagtgagacaagcaggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttgtcacagtatcggcatctctgtcgccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctattgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaacaaatataaaacaatgaccacggctcaaatattgggtaatcttttccctagtgccggtgatatccctgaaaaatatagattggacggtcagattgaacaaagagaatatttggttgatggagagctaaaaacttggccaggtgccttagcccaagtaagaagccctgtttacttggccggtgctaacggagatgaacaagtaatcttaggctccacgcctctgttggacgccgataccgcgcttaccgctttagatgctgcagtcagggcttacgatcgtggtcaaggtttgtggcctactatgagggtggctgaacgtatacaacatgtagaaacgtttttaggccacatgagagaacaaagggaagcggtggtaaaattgttaatgtgggaaattggaaagaacttgaaagattccgaaaaggaatttgatagaacatgcgattatatagttgatactgtgaacgcattaaaggaattagatagaagatcatcaaggttcgaattagaacaggatacactgggtcaaatccgtagggttccaatgggtgtagctctatgtatgggtccatacaactaccctctgaacgaaacattcactaccttaattcctgccctaattatgggtaatacagtggtttttaagccagctaagcttggagttttgttgattagaccgttgttggaagcatttcgtgattccttccccgctggagtgatcaatgttatctacggttccggtagggaaactgtatctgctctgatggcaagtggtaaaatcgatatattcgcttttataggaactaacaaagcagcctccgatttaaaaaaattgcaccctagaccacatagattgagggcagcactaggtctggacgctaaaaacccaggaatcattttacccgaagttgatttagataatgcagttaatgaggctgtcactggttctctttcattcaacggacagagatgcactgctttgaagattctttttgtacatgaggatgtggtcgaggcctttatcgacaaatttaacgcaaaattggcaacccttaaaccaggcatgccatgggactccggtgttgcattaactccgttgcctgaagctagtaaagtggactatctacattcacttgtagccgacgctgtttcgaaaggagccgccgttgtaaacccgaacggtggtgaagcgcgtgcttcattcttttatccagctgtcctgtaccctgtgactcctcaaatgagagtctatcaagaagagcagtttgggcctgtagttccaatcgttccgtatagacacttagacacggttatagattacgtgttggaatcagattttggccaacaattgagcatcttcggaactaatccagttgcggtcggtaggctggttgacacatttgcaaatcaggtaggaagaataaatctaaatgcacagtgtcagagggggccggatacgtatcccttcaacggccgtaaaaattccgccgagggaaccttgagtgtgcacgacgcgttaagagtcttttccataagaaccttagtagccactaagtttcaagaaactaataaagaactgatctctgatatcatctccggtagatcctcaagctttcttacgacagattacattttttaacgcgaatttcttatgatttatgatttttattattaaataagttataaaaaaaataagtgtatacaaattttaaagtgactcttaggttttaaaacgaaaattcttattcttgagtaactctttcctgtaggtcaggttgctttctcaggtatagcatgaggtcgctcttattgaccacacctctaccggcatgccgagcaaatgcctgcaaatcgctccccatttcacccaattgtagatatgctaactccagcaatgagttgatgaatctcggtgtgtattttatgtcctcagaggacaacacgcttgaagactgagtccgtctcatacccacagtatcggcatctctgtccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaaaatgacgctatctaccaagccgaaagtcttgaagctaggtgacttcgagttcagtcaagcaaggtgggatgaactgtcccagattgccgaggtcatcaactgcgaatctcagaatagggatgaattcatcaacgatttgcaaaccaaatacaacgatataacctatattgccagggaatataacagcgttaatgttactggaaggttcgacgcagagatagcacaacatatccccagcactgttaaggggatctgccattgcaccgctggttacgactctatcgatgttgagcccttcagcaagttggggattcagattagtaatgttacggatccagtggtaggacctactgctgacgttgcagtattccttgtgttagcttgtcttaggaatttctttgcaggtcataaaatgttggtatccgggaaatggcctgatgagaaggttcttgagtgtggcggagttgacatgggtaacactcctcaacataaggtactaggaatcttaggtatgggaaacattgggcgtgccgtgaagaataggttgaaagtattcggattcaagaagatcgtatactataatagaaggcaactgtcgccagagctggaagagggcgccgagtacgtaagtcaggacgagttgttgcagaaatccgatgttattgtcgtcgttgttcccttaaatccaaacacgaagcatctaatagaccgtaacgcgatatctaagatgaaggacggagtaatattggtgaacatttctcgtggattggtcttggatgaacacgttgtcacggagttagtgaaatccggcaagataggtgcctttggtgcagacgttttcgagaacgagccgcacgtctcacctgacctttacaacctgcctaacgtggtaacattgccgcacatggggacgcacgcgtttgacgcaactcaggacttgcaggagtgggtgattgagaatattgaatgctgtataaagactggaaaactgaaaactctggtcgcggaacagaaggagctgttctaataacacaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgcttgaagactgagtccgtctcagcgagctcttctagaataacttcgtataatgtatgctatacgaagttatccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagagcgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaaatgccacaatcctgggaagaattggccgccgacaaacgtgcccgtttggctaaaaccattcctgacgaatggaaggttcaaactttgcctgccgaagattccgttattgatttcccaaagaagtccggtattttgtctgaggctgaattgaagattaccgaagcctctgctgctgatttggtttccaagttggccgctggtgagttgacttctgttgaagtcactttggctttttgtaagagagctgctattgctcaacaattaaccaactgtgctcacgaattcttcccagatgctgctttagctcaagctagagaattagatgaatactacgctaagcataaaagaccagttggtccattacacggtttaccaatctctttaaaggaccaattgcgtgttaagggttacgaaacctccatgggttacatttcctggttaaacaaatacgatgaaggtgattccgtcttaaccaccatgttgagaaaagctggtgctgttttctacgttaagacctctgtcccacaaaccttgatggtctgtgaaaccgtcaacaacatcattggtagaactgtcaatccaagaaacaaaaattggtcctgtggtggttcttctggtggtgaaggtgctattgttggtattagaggtggtgttattggtgtcggtactgacattggtggttccattagagtcccagctgctttcaactttttatacggtttgcgtccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttgcaacgc

SEQ ID NO: 119 - DNA, 1363 bp, synthetic construct
  source 1..1363: mol_type = other DNA
agaggtggtgttattggtgtcggtactgacattggtggttccattagagtcccagctgctttcaactttttatacggtttgagaccatctcacggtagattgccatatgctaaaatggctaactctatggaaggtcaagaaaccgttcactccgtcgttggtcctatcactcactccgtcgaagacttgagattgttcaccaaatctgtcttgggtcaagaaccttggaagtacgactctaaggtcatccccatgccatggagacaatctgaatctgacatcattgcctctaagattaagaatggtggtttgaacattggttattacaatttcgacggtaacgtcttgccacacccaccaattttacgtggtgtcgaaactaccgttgccgctttggccaaggctggtcacaccgttactccatggactccatacaagcatgatttctgtcatgacttgatttcccacatctatgctgctgatggttctgccgacgtcatgagagacatttctgcctctggtgagccagccatccctaacattaaggacttgttgaacccaaatattaaggctgttaacatgaacgaattgtgggacactcatttacaaaagtggaactatcaaatggaatacttggaaaagtggcgtgaagctgaagaaaaagctggtaaggaattggacgctattatcgctccaattactcctaccgtcgctgtcagacacgatcaattcagatactacggttacgcctccgttattagcttattggatttcacctctgttgtcgtcccagtcactttcgctgataagaatattgataagaagaacgaatcttttaaagctgtttccgaattggatgctttggttcaagaagaatacgacccagaggcttatcacggtgctcctgttgctgttcaagttattggtagaagattgtccgaagagagaactttggctatcgccgaagaagtcggtaaattgttgggtaacgtcgtcactccataaggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcgataacttcgtataatgtatgctatacgaagttatgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtc

SEQ ID NO: 120 - protein, 250 aa, Saccharomyces cerevisiae
MPLTTKPLSLKINAALFDVDGTIIISQPAIAAFWRDFGKDKPYFDAEHVIHISHGWRTYDAIAKFAPDFADEEYVNKLEGEIPEKYGEHSIEVPGAVKLCNALNALPKEKWAVATSGTRDMAKKWFDILKIKRPEYFITANDVKQGKPHPEPYLKGRNGLGFPINEQDPSKSKVVVFEDAPAGIAAGKAAGCKIVGIATTFDLDFLKEKGCDIIVKNHESIRVGEYNAETDEVELIFDDYLYAKDDLLKW

SEQ ID NO: 121 - protein, 250 aa, Saccharomyces cerevisiae
MGLTTKPLSLKVNAALFDVDGTIIISQPAIAAFWRDFGKDKPYFDAEHVIQVSHGWRTFDAIAKFAPDFANEEYVNKLEAEIPVKYGEKSIEVPGAVKLCNALNALPKEKWAVATSGTRDMAQKWFEHLGIRRPKYFITANDVKQGKPHPEPYLKGRNGLGYPINEQDPSKSKVVVFEDAPAGIAAGKAAGCKIIGIATTFDLDFLKEKGCDIIVKNHESIRVGGYNAETDEVEFIFDDYLYAKDDLLKW

SEQ ID NO: 122 - protein, 515 aa, Saccharomycopsis fibuligera
MIRLTVFLTAVFAAVASCVPVELDKRNTGHFQAYSGYTVARSNFTQWIHEQPAVSWYYLLQNIDYPEGQFKSAKPGVVVASPSTSEPDYFYQWTRDTAITFLSLIAEVEDHSFSNTTLAKVVEYYISNTYTLQRVSNPSGNFDSPNHDGLGEPKFNVDDTAYTASWGRPQNDGPALRAYAISRYLNAVAKHNNGKLLLAGQNGIPYSSASDIYWKIIKPDLQHVSTHWSTSGFDLWEENQGTHFFTALVQLKALSYGIPLSKTYNDPGFTSWLEKQKDALNSYINSSGFVNSGKKHIVESPQLSSRGGLDSATYIAALITHDIGDDDTYTPFNVDNSYVLNSLYYLLVDNKNRYKINGNYKAGAAVGRYPEDVYNGVGTSEGNPWQLATAYAGQTFYTLAYNSLKNKKNLVIEKLNYDLYNSFIADLSKIDSSYASKDSLTLTYGSDNYKNVIKSLLQFGDSFLKVLLDHIDDNGQLTEEINRYTGFQAGAVSLTWSSGSLLSANRARNKLIELL

SEQ ID NO: 123 - protein, 604 aa, Rhizopus delemar
MQLFNLPLKVSFFLVLSYFSLLVSAASIPSSASVQLDSYNYDGSTFSGKIYVKNIAYSKKVTVIYADGSDNWNNNGNTIAASYSAPISGSNYEYWTFSASINGIKEFYIKYEVSGKTYYDNNNSANYQVSTSKPTTTTATATTTTAPSTSTTTPPSSSEPATFPTGNSTISSWIKKQEGISRFAMLRNINPPGSATGFIAASLSTAGPDYYYAWTRDAALTSNVIVYEYNTTLSGNKTILNVLKDYVTFSVKTQSTSTVCNCLGEPKFNPDGSGYTGAWGRPQNDGPAERATTFILFADSYLTQTKDASYVTGTLKPAIFKDLDYVVNVWSNGCFDLWEEVNGVHFYTLMVMRKGLLLGADFAKRNGDSTRASTYSSTASTIANKISSFWVSSNNWIQVSQSVTGGVSKKGLDVSTLLAANLGSVDDGFFTPGSEKILATAVAVEDSFASLYPINKNLPSYLGNSIGRYPEDTYNGNGNSQGNPWFLAVTGYAELYYRAIKEWIGNGGVTVSSISLPFFKKFDSSATSGKKYTVGTSDFNNLAQNIALAADRFLSTVQLHAHNNGSLAEEFDRTTGLSTGARDLTWSHASLITASYAKAGAPAA

SEQ ID NO: 124 - protein, 599 aa, Rhizopus oryzae
MKFISTFLTFILAAVSVTAGASIPSSASVQLDSYNYDGSTFSGKIYVKNIAYSKKVTVVYADGSDNWNNNGNTIAASFSGPISGSNYEYWTFSASVKGIKEFYIKYEVSGKTYYDNNNSANYQVSTSKPTTTTAATTTTTAPSTSTTTRPSSSEPATFPTGNSTISSWIKKQEDISRFAMLRNINPPGSATGFIAASLSTAGPDYYYAWTRDAALTSNVIVYEYNTTLSGNKTILNVLKDYVTFSVKTQSTSTVCNCLGEPKFNPDGSGYTGAWGRPQNDGPAERATTFVLFADSYLTQTKDASYVTGTLKPAIFKDLDYVVNVWSNGCFDLWEEVNGVHFYTLMVMRKGLLLGADFAKRNGDSTRASTYSSTASTIANKISSFWVSSNNWVQVSQSVTGGVSKKGLDVSTLLAANLGSVDDGFFTPGSEKILATAVAVEDSFASLYPINKNLPSYLGNAIGRYPEDTYNGNGNSQGNPWFLAVTGYAELYYRAIKEWISNGGVTVSSISLPFFKKFDSSATSGKKYTVGTSDFNNLAQNIALAADRFLSTVQLHAPNNGSLAEEFDRTTGFSTGARDLTWSHASLITASYAKAGAPAA

SEQ ID NO: 125 - DNA, 682 bp, Saccharomyces cerevisiae
  source 1..682: mol_type = genomic DNA
atctcccgagtttatcattatcaatactgccatttcaaagaatacgtaaataattaatagtagtgattttcctaactttatttagtcaaaaaattggccttttaattctgctgtaacccgtacatgcccaaaatagggggcgggttacacagaatatataacatcataggtgtctgggtgaacagtttattcctggcatccactaaatataatggagcccgctttttttaagctggcatccagaaaaaaaaagaatcccagcaccaaaatattgttttcttcaccaaccatcagttcataggtccattctcttagcgcaactacacagaacaggggcacaaacaggcaaaaaacgggcacaacctcaatggagtgatgcaacctgcttggagtaaatgatgacacaaggcaattgacctacgcatgtatctatctcattttcttacaccttctattaccttctgctctctctgatttggaaaaagctgaaaaaaaaggttgaaaccagttccctgaaattattcccctatttgactaataagtatataaagacggtaggtattgattgtaattctgtaaatctatttcttaaacttcttaaattctacttttatagttagtcttttttttagtttaaaacaccaagaacttagtttcgaataaacacacataaacaaacaaa

SEQ ID NO: 126 - DNA, 507 bp, Saccharomyces cerevisiae
  source 1..507: mol_type = genomic DNA
ggcaacggttcatcatctcatggatctgcacatgaacaaacaccagagtcaaacgacgttgaaattgaggctactgcgccaattgatgacaatacagacgatgataacaaaccgaagttatctgatgtagaaaaggattagagatgctaagagatagtgatgatatttcataaataatgtaattctatatatgttaattaccttttttgcgaggcatatttatggtgaaggataagttttgaccatcaaagaaggttaatgtggctgtggtttcagggtccataaagcttttcaattcatcttttttttttttgttcttttttttgattccggtttctttgaaatttttttgattcggtaatctccgagcagaaggaagaacgaaggaaggagcacagacttagattggtatatatacgcatatgtggtgttgaagaaacatgaaattgcccagtattcttaacccaactgcacagaacaaaaacctgcaggaaacgaagataaatc

SEQ ID NO: 127 - DNA, 587 bp, Saccharomyces cerevisiae
  source 1..587: mol_type = genomic DNA
ggccagaaaaaggaagtgtttccctccttcttgaattgatgttaccctcataaagcacgtggcctcttatcgagaaagaaattaccgtcgctcgtgatttgtttgcaaaaagaacaaaactgaaaaaacccagacacgctcgacttcctgtcttcctgttgattgcagcttccaatttcgtcacacaacaaggtcctagcgacggctcacaggttttgtaacaagcaatcgaaggttctggaatggcgggaaagggtttagtaccacatgctatgatgcccactgtgatctccagagcaaagttcgttcgatcgtactgttactctctctctttcaaacagaattgtccgaatcgtgtgacaacaacagcctgttctcacacactcttttcttctaaccaagggggtggtttagtttagtagaacctcgtgaaacttacatttacatatatataaacttgcataaattggtcaatgcaagaaatacatatttggtcttttctaattcgtagtttttcaagttcttagatgctttctttttctcttttttacagatcatcaaggaagtaattatctactttttacaag

SEQ ID NO: 128 - DNA, 456 bp, Saccharomyces cerevisiae
  source 1..456: mol_type = genomic DNA
ccttacatcacacccaatcccccacaagtgatcccccacacaccatagcttcaaaatgtttctactccttttttactcttccagattttctcggactccgcgcatcgccgtaccacttcaaaacacccaagcacagcatactaaatttcccctctttcttcctctagggtggcgttaattacccgtactaaaggtttggaaaagaaaaaagagaccgcctcgtttctttttcttcgtcgaaaaaggcaataaaaatttttatcacgtttctttttcttgaaaaattttttttttgatttttttctctttcgatgacctcccattgatatttaagttaataaatggtcttcaatttctcaagtttcagtttcgtttttcttgttctattacaactttttttacttcttgctcattagaaagaaagcatagcaatctaatctaagttttaattacaaa

SEQ ID NO: 129 - DNA, 577 bp, Saccharomyces cerevisiae
  source 1..577: mol_type = genomic DNA
taacacttacagagtcctatcaggaaagaataaaagccgatcaagcttcattctcaggtaacaactggagcactggcgagctttgctctactatataagataaaatatgcactaaaagtttgcatttctttacataactaaaactaagacattatgcatagcttacctgatcaaaaagtatgtaaacttgttaacatcttcacatgtgattcatctggtcgtactttcttgcggtgcagtgtaatatttctacccacgtgactataattgagcttgaaaactgtggcgtttttccaccgatgggtccacgccagatattaaccgaagccaaaataccgatgaaatttctgagatagctcttgtaaacgacgtcaaatcttcatatgcaaggagatcttgatttctttttggtagtcatctgtcgtcttgaggcgtataagaaggaggttatatctgtcctttctacaaagtattttcgagaatcttgcctctgccccttttttctttttttaaaaggtttaaaaaacataactgtcttcaatatatccagtatttacgacaatatacaaacataatc

SEQ ID NO: 130 - DNA, 747 bp, Saccharomyces cerevisiae
  source 1..747: mol_type = genomic DNA
gatctcccctaaaccgtggaatatttcggatatccttttgttgtttccgggtgtacaatatggacttcctcttttctggcaaccaaacccatacatcgggattcctataataccttcgttggtctccctaacatgtaggtggcggaggggagatatacaatagaacagataccagacaagacataatgggctaaacaagactacaccaattacactgcctcattgatggtggtacataacgaactaatactgtagccctagacttgatagccatcatcatatcgaagtttcactaccctttttccatttgccatctattgaagtaataataggcgcatgcaacttcttttctttttttttcttttctctctcccccgttgttgtctcaccatatccgcaatgacaaaaaaatgatggaagacactaaaggaaaaaattaacgacaaagacagcaccaacagatgtcgttgttccagagctgatggggggtatctcgaagcacacgaaactttttccttccttcattcacgcacactactctctaatgagcaacggtatacggccttccttccagttacttgaatttgaaataaaaaaagtttgctgtcttgctatcaagtataaatagacctgcaattattaatcttttgtttcctcgtcattgttctcgttccctttcttccttgtttctttttctgcacaatatttcaagctataccaagcatacaatcaactatctcatataca

SEQ ID NO: 131 - DNA, 224 bp, Saccharomyces cerevisiae
  source 1..224: mol_type = genomic DNA
acaggccccttttcctttgtcgatatcatgtaattagttatgtcacgcttacattcacgccctcctcccacatccgctctaaccgaaaaggaaggagttagacaacctgaagtctaggtccctatttatttttttatagttatgttagtattaagaacgttatttatatttcaaatttttcttttttttctgtacaaacgcgtgtacgcatgtaacgggcagac

SEQ ID NO: 132 - DNA, 320 bp, Saccharomyces cerevisiae
  source 1..320: mol_type = genomic DNA
aaactgtattataagtaaatgcatgtatactaaactcacaaattagagcttcaatttaattatatcagttattacccgggaatctcggtcgtaatgatttttataatgacgaaaaaaaaaaaattggaaagaaaaagcttcatggcctttataaaaaggaaccatccaatacctcgccagaaccaagtaacagtattttacggggcacaaatcaagaacaataagacaggactgtaaagatggacgcattgaactccaaagaacaacaagagttccaaaaagtagtggaacaaaagcaaatgaaggatttcatgcgtttg

SEQ ID NO: 133 - DNA, 471 bp, Saccharomyces cerevisiae
  source 1..471: mol_type = genomic DNA
tttaccagcttactatccttcttgaaaatatgcactctatatcttttagttcttaattgcaacacatagatttgctgtataacgaattttatgctatttttttaatttggagttcggtgatgaaagtgtcacagcgaatttcctcacatgtagggaccgaattgtttacaagttctctgtaccaccatggagacatcaaagattgaaaatctatggaaagatatggacggtagcaacaagaatatagcacgagccgcggagttcatttcgttacttttgatatcgctcacaactattgcgaagcgcttcagtgaaaaaatcataaggaaaagttgtaaatattattggtagtattcgtttggtaaagtagagggggtaatttttcccctttattttgttcatacattcttaaattgctttgcctctccttttggaaagctatacttcggagcactgttgagcgaaggctca

SEQ ID NO: 134 - DNA, 303 bp, Saccharomyces cerevisiae
  source 1..303: mol_type = genomic DNA
gacaataacaactctgcaaactaccaagtctcaacttctaaacctactacaactactgcagctacaaccacaactacagctccatcaacttctacaacaacccgtccatctagttcagagcctgccaccttccctactggtaattctaccatcagctcttggatcaaaaagcaggaagatatttccagattcgctatgcttagaaacatcaacccacctggttctgccacagggtttatcgccgcatcactctctaccgctggtccagattactactacgcgtggacaagagatgccgctttg

SEQ ID NO: 135 - DNA, 160 bp, Saccharomyces cerevisiae
  source 1..160: mol_type = genomic DNA
ggagattgataagacttttctagttgcatatcttttatatttaaatcttatctattagttaattttttgtaatttatccttatatatagtctggttattctaaaatatcatttcagtatctaaaaattcccctcttttttcagttatatcttaacaggcg

SEQ ID NO: 136 - DNA, 500 bp, Saccharomyces cerevisiae
  source 1..500: mol_type = genomic DNA
ggaaatccgtatcattttctcgcatacacgaacccgcgtgcgcctggtaaattgcaggattctcattgtccggttttctttatgggaataatcatcatcaccattatcactgttactcttgcgatcatcatcattaacataatttttttaacgctgtttgatgatggtatgtgcttttattgttccttactcaccttttcctttgtgtcttttaattttgaccattttgaccattttgacctttgatgatgtgtgagttcctcttttctttttttcttttcttttttcctttttttttcttttcttactgtgttaatcactttctttcctttttgttcatattgtcgtcttgttcattttcgttcaattgataatgtatataaatctttcgtaagtatctcttgattgccatttttttctttccaagtttccttgttatgaaacgtttcaatgttttaaaatatatcagaacaacaaaagcaaatatacaaaccatcgca

SEQ ID NO: 137 - DNA, 500 bp, Saccharomyces cerevisiae
  source 1..500: mol_type = genomic DNA
ttttcttttatttttttgataaaactactacgctaaaaataaaataaaaatgtatgatttccctccatttccgaccaattgtataattttatatctgcatgacttaataatataatataatacttataaaatacgaatagaaaaatttaaaccgatgtaatgcatccttttctttgttgtcttcggatgatctgccgtgacaggtggttcgcgcaaatcaaactggtttagagaatttaacacagaaataaaaaaggaagattcaatcttcgtttttgttttatatcttactataaaagtgttttttttttagtacgacgagaagttagagtaattataaaaggaatgcttatttaaatttatttcttagacttcttttcagacttcttagcagcctcagtttgttccttaacgaccttcttaacaatcttttgttcttcaatcaagaaagctctgacgattctttccttgacacagttggcacatctggaaccaccgta

SEQ ID NO: 138 - DNA, 500 bp, Saccharomyces cerevisiae
  source 1..500: mol_type = genomic DNA
aagccaccgctctgtcagggtaaccctatgaaacatttcaaaacgttataaaggaactcgtctggttacaacaaggaactatcactacaaacagctgtccgtgcggctcctcaactctctcaatgttgttcgcctggtcacacacagcatagtttcgtcattcggcgccgacggtcgctgtctcttggagccttcaagctcttgtcaacccaggtccgttgtgccgataaaagtaacagcagcccccccacgcccgcatcccactctcttctccgaccacctccctcgaagttcttccctgccaatcccacgtcgatccagcgtagttggccccaactggtgcagtaataaccgcttagcgattttgcactcggaactacatatgtatatatatatgtgtgtgtgtgtgtgggctggaaagatttcttgagcttccgtgttatagtgcaatttaaatattgtacatcattccgatccagctggaaacaaaagcaagaaca

SEQ ID NO: 139 - DNA, 500 bp, Saccharomyces cerevisiae
  source 1..500: mol_type = genomic DNA
gtatctgattttcctttttctcccttcacgtaaacctgaaatatatttcatgtaatatatatagttcatccttaaagaaaacataccatttcaaaatgccctttcccagtagctaatacatgcttaaaaatgtgatatccattattaccatcaaatgtattcatgatccgggtagtgcctttttttccttcaaacactaatgggatgggaagaaacatttcgaaaaaagttatgactattggcagcatcagagagaattaaaccttacctgtacaacgatagcagcttacatcacttcctcgcagtcatggaaaaaaatacggcaccatttgtcgtcgatacggcacccacaactcctccagataaacttgtagcaccttctatagaggaagtaaatagtaatcccaacgaattaagagctcttagagggcagggtagatattttggcgtaagcgatgatgacaaggatgccattaaagaggcagcacctaaatgtaata

SEQ ID NO: 140 - DNA, 500 bp, Saccharomyces cerevisiae
  source 1..500: mol_type = genomic DNA
aaaaagaagaaaaacagaaggccaagacagggtcaatgagactgttgtcctcctactgtccctatgtctctggccgatcacgcgccattgtccctcagaaacaaatcaaacacccacaccccgggcacccaaagtccccacccacaccaccaatacgtaaacggggcgccccctgcaggccctcctgcgcgcggcctcccgccttgcttctctcccctcccttttctttttccagttttccctattttgtccctttttccgcacaacaagtatcagaatgggttcatcaaatctatccaacctaattcgcacgtagactggcttggtattggcagtttcgcagttatatatatactaccatgagtgaaactgttacgttaccttaaattctttctccctttaattttcttttatcttactctcctacataagacatcaagaaacaattgtatattgtacacccccccccctccacaaacacaaatattgataatataaag

SEQ ID NO: 141 - DNA, 500 bp, Saccharomyces cerevisiae
  source 1..500: mol_type = genomic DNA
atttattggagaaagataacatatcatactttcccccacttttttcgaggctcttctatatcatattcataaattagcattatgtcatttctcataactactttatcacgttagaaattacttattattattaaattaatacaaaatttagtaaccaaataaatataaataaatatgyatatttaaattttaaaaaaaaatcctatagagcaaaaggattytccattataatatgagctatacacctcttacgcattttttgagggtggttacaacaccactcattcagaggctgtcggcacagttgcttccagcatctggcgtccgtatgtatgggtgtattttaaataataaacaaagtgccacaccttcaccaattatgtctttaagaaatggacaagttccaaagagcttgcccaaggctcgacraggatgtactttagaatatctatattcaagtacgtggcgcgcatatgtttgagtgtgcacacaataaaggt

(19) United States
(12) Patent Application Publication
(10) Pub. No.: US 2026/0297503 A1
(43) Pub. Date: Oct. 1, 2026

(54) GENETICALLY MODIFIED YEAST AND FERMENTATION PROCESSES FOR THE PRODUCTION OF ETHANOL

(71) Applicant: CARGILL, INCORPORATED, Wayzata, MN (US)

(72) Inventors: Christopher Kenneth Miller, Andover, MN (US); Gregory Michael Poynter, St. Paul, MN (US)

(73) Assignee: CARGILL, INCORPORATED, Wayzata, MN (US)

(21) Appl. No.: 18/998,233

(22) PCT Filed: Aug. 11, 2023
(86) PCT No.: PCT/US2023/072055

§ 371 Date: Jan. 24, 2025

Related U.S. Application Data
(60) Provisional application No. 63/371,692, filed on Aug. 17, 2022.

(51) Int. Cl. C12N 1/18; C12N 9/02; C12N 9/04; C12N 9/16; C12N 9/34; C12P 7/06; C12R 1/865

(52) U.S. Cl. CPC C12N 1/18; C12N 9/0006; C12N 9/0008; C12N 9/16; C12N 9/2428; C12P 7/06; C12Y 101/01001; C12Y 102/01012; C12Y 301/03021; C12Y 302/01003; C12N 2830/34; C12N 2830/36; C12R 2001/865

(57) ABSTRACT

Disclosed herein are genetically engineered yeast cells capable of producing ethanol. The genetically engineered yeast cells comprise an exogenous polynucleotide sequence encoding a glyceraldehyde-3-phosphate dehydrogenase (gapN) enzyme at least 60%, at least 70%, at least 80%, at least 85%, at least 90%, at least 95%, at least 97%, at least 99%, or 100% identical to at least one of SEQ ID NOs: 28, 32, 48, 52, 64, 68, 80, 92, and 96.

### CROSS-REFERENCE TO RELATED APPLICATIONS

This application claims the benefit of U.S. Provisional Application No. 63/371,692, filed Aug. 17, 2022, which is incorporated by reference herein in its entirety.

### REFERENCE TO A SEQUENCE LISTING SUBMITTED VIA PATENT CENTER

The content of the Sequence Listing XML file of the sequence listing named “PT-1298-WO-PCT.xml” which is 479,867 bytes in size created on Aug. 3, 2023 and electronically submitted via Patent Center herewith the application is incorporated by reference in its entirety.

### BACKGROUND

Many fermentation feedstocks are derived from plant sources (e.g., corn mash) where the carbohydrates are predominantly in the form of starch polymers. The starch polymers in such feedstocks must be treated to low molecular weight sugars that can be consumed by the yeast and used for growth and bioproduct production. Typical treatments include acid and/or enzymatic hydrolysis where the polymer chain is hydrolyzed to generate the sugars that can be used by the yeast. Starch degrading enzymes such as alpha amylases and glucoamylases can be added to convert the polymer to simple sugars. However, such enzyme additions can add significant cost and complexity to the fermentation process.

Heterologous expression and functionality of enzymes in yeast to aid in starch hydrolysis can be challenging, as it is difficult to know if the nucleic acid will be expressed properly and a functional enzyme will form, and if an active form of the enzyme will be secreted from the cell. It is also challenging to engineer yeast for growth and bioproduct production at non-optimal conditions, such as high temperatures, and in high bioproduct titers. For example, while ethanol production by fermentation is a well know industrial process, maintaining ethanol rates, titers, and yields while at the same time engineering the yeast to reduce reliance on supplemental enzymes, growth under non-optimal conditions (e.g., temperature), and minimizing by-product formation can be technically difficult. Increased ethanol concentration and accumulation of undesirable byproducts can also be detrimental to cell health.

### SUMMARY

The present disclosure provides a genetically engineered yeast cell capable of producing ethanol, the engineered yeast cell comprising an exogenous polynucleotide encoding a glyceraldehyde-3-phosphate dehydrogenase (gapN) enzyme at least 60%, at least 70%, at least 80%, at least 85%, at least 90%, at least 95%, at least 97%, at least 99%, or 100% identical to at least one of SEQ ID NOs: 28, 32, 48, 52, 64, 68, 80, 92, and 96. The gapN enzyme may be at least 60%, at least 70%, at least 80%, at least 85%, at least 90%, at least 95%, at least 97%, at least 99%, or 100% identical to at least one of SEQ ID NOs: 28, 32, 48, 52, and 64. The gapN enzyme may be at least 60%, at least 70%, at least 80%, at least 85%, at least 90%, at least 95%, at least 97%, at least 99%, or 100% identical to at least one of SEQ ID NOs: 28, 32, 52, and 64. the gapN enzyme may be at least 85% identical to SEQ ID NO:28; the gapN enzyme may be at least 85% identical to SEQ ID NO:32; the gapN enzyme may be at least 85% identical to SEQ ID NO:52; and/or the gapN enzyme may be at least 85% identical to SEQ ID NO:64. The engineered yeast cell may be capable of producing ethanol at a titer of at least 60, at least 80, at least 100, or at least 120 g/L ethanol after 48 hours and wherein glycerol production by the engineered yeast cell is reduced relative to glycerol production in an equivalent yeast cell lacking the gapN enzyme.

The engineered yeast cell may additionally comprise an exogenous polynucleotide sequence encoding an alcohol dehydrogenase (ADH) enzyme at least 60%, at least 70%, at least 80%, at least 85%, at least 90%, at least 95%, at least 97%, at least 99%, or 100% identical to at least one of SEQ ID NOs: 98 and 100. The engineered yeast cell may comprise a deletion or disruption of a native glycerol-3-phosphate phosphatase (GPP) gene. The engineered yeast cell may comprise a deletion or disruption of a native glycerol-3-phosphate dehydrogenase (GDP) gene. The engineered yeast cell may additionally comprise an exogenous polynucleotide sequence encoding a glucoamylase (GA) enzyme. The encoded GA enzyme may be at least 60%, at least 70%, at least 80%, at least 85%, at least 90%, at least 95%, at least 97%, at least 99%, or 100% identical to at least one of SEQ ID NOs: 1, 122, 123, and 124. The engineered yeast cell may be capable of producing ethanol at a titer of at least 60, at least 80, at least 100, or at least 120 g/L ethanol after 48 hours and wherein glycerol production by the engineered yeast cell is reduced relative to glycerol production in an equivalent yeast cell lacking the gapN enzyme.

Also provided is a genetically engineered yeast cell capable of producing ethanol, the engineered yeast cell comprising an exogenous polynucleotide encoding a glyceraldehyde-3-phosphate dehydrogenase (gapN) enzyme at least 60%, at least 70%, at least 80%, at least 85%, at least 90%, at least 95%, at least 97%, at least 99%, or 100% identical to at least one of SEQ ID NOs: 28, 32, 48, 52, 64, 68, 80, 92, and 96; an exogenous polynucleotide sequence encoding an alcohol dehydrogenase (ADH) enzyme at least 60%, at least 70%, at least 80%, at least 85%, at least 90%, at least 95%, at least 97%, at least 99%, or 100% identical to at least one of SEQ ID NOs: 98 and 100; an exogenous polynucleotide sequence encoding a glucoamylase (GA) enzyme; and a deletion or disruption of at least 1 allele of a native GPP gene. The encoded GA enzyme may be at least 60%, at least 70%, at least 80%, at least 85%, at least 90%, at least 95%, at least 97%, at least 99%, or 100% identical to at least one of SEQ ID NOs: 1, 122, 123, and 124.

One or more of the exogenous polynucleotide sequences may be operably linked to a heterologous or artificial promoter. The promoter may be selected from the group consisting of a pyruvate decarboxylase (PDC) promoter, a glyceraldehyde-3-phosphate dehydrogenase GAPDH (TDH3) promoter, a translation elongation factor 1 (TEF1) promoter, a URA3 promoter, an S-adenosyl methionine transferase 2 (SAM2) promoter; an alcohol dehydrogenase 1 (ADH1) promoter, and a 3-phosphoglycerate kinase (PGK1) promoter. One or more of the exogenous polynucleotide sequences may be operably linked to a heterologous or artificial terminator. The terminator may be selected from the group consisting of an iso-1-cytophrome c (CYC1) terminator, a URA3 terminator, a PDC terminator, an ADHI terminator, a TEF1 terminator, or a GAL10 terminator.

The yeast cell may be selected from the group consisting of Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., and Yarrowia spp. The yeast cell may be a Saccharomyces cerevisiae cell.

The disclosure also provides a method for producing ethanol, the method comprising contacting a substrate with an engineered yeast as described herein, where the engineered yeast cell produces at least 60, at least 80, at least 100, or at least 120 g/L ethanol after 48 hours and wherein glycerol production by the engineered yeast cell is reduced relative to glycerol production in an equivalent yeast cell lacking the gapN enzyme. The substrate may comprise starch, glucose, sucrose, cellulosic biomass, or combinations thereof. The substrate may be obtained from wheat, corn, or a combination thereof.

### BRIEF DESCRIPTION OF THE FIGURES

The drawings illustrate generally, by way of example, but not by way of limitation, various aspects discussed herein.

FIG. 1 shows show screening of gapN biodiversity as outlined in Example 1.

FIG. 2 shows glycerol production in the deep well plate assays outlined in Example 3.

FIG. 3 shows ethanol production in the deep well plate assays outlined in Example 3.

FIG. 4 shows NADP redox balancing using a combination of an NADP dependent gapN and an NADP dependent ADH enzyme in the production of ethanol from glucose.

FIG. 5 shows the results of the ADH enzyme assay outlined in Example 4.

FIG. 6 shows the glycerol and ethanol titers from shake flask assays outlined in Example 6.

FIG. 7 shows the glycerol and ethanol titers from shake flask assays outlined in Example 6.

FIG. 8 shows the glycerol and ethanol titers from shake flask assays outlined in Example 6.

FIG. 9 shows the glycerol and ethanol titers from the Ambr15 assays outlined in Example 7.

### DETAILED DESCRIPTION

Reference will now be made in detail to certain aspects of the disclosed subject matter, examples of which are illustrated in part in the accompanying drawings. While the disclosed subject matter will be described in conjunction with the enumerated claims, it will be understood that the exemplified subject matter is not intended to limit the claims to the disclosed subject matter.

In this document, the terms “a,” “an,” or “the” are used to include one or more than one unless the context clearly dictates otherwise. The term “or” is used to refer to a nonexclusive “or” unless otherwise indicated. All publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

Values expressed in a range format should be interpreted in a flexible manner to include not only the numerical values explicitly recited as the limits of the range, but also to include all the individual numerical values or sub-ranges encompassed within that range as if each numerical value and sub-range were explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

Unless expressly stated, ppm (parts per million), percentage, and ratios are on a by weight basis. Percentage on a by weight basis is also referred to as wt % or % (wt) below.

This disclosure relates to various recombinant cells engineered to produce ethanol via glucose, said recombinant cells also having reduced glycerol production. In general, the recombinant cells described herein include a heterologous nucleic acid encoding a gapN enzyme, for example the gapN enzyme of at least one of SEQ ID NOs: 28, 32, 48, 52, 64, 68, 80, and 92. The recombinant cell may additionally include a heterologous nucleic acid encoding an ADH enzyme, for example, the ADH enzyme of at least one of SEQ ID NO:99 and 100. The disclosure further provides fermentation methods for the production of ethanol using the genetically engineered cells described herein.

In general, recombinant cells described herein are yeast cells. Non-limiting examples of yeast cells include yeast cells obtained from, e.g., Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., Yarrowia spp, and industrial polyploid yeast strains. Suitable yeast cells may include, but are not limited to, Saccharomyces cerevisiae, Issatchenkia orientalis, Pichia galeiformis, Pichia sp. YB-4149 (NRRL designation), Candida ethanolica, Pichia deserticola, Pichia membranifadens, or Pichia fermentans. The yeast cell may be an ethanol tolerant yeast strain, for example, a commercially available ethanol tolerance yeast such as RED STAR™ and ETHANOL RED™ yeast (Fermentis/Lesaffre, USA), FALI™ (Fleischmann's Yeast, USA), SUPERSTART and THERMOSACC™ yeast (Ethanol Technology, Wis., USA), BIOFERM™ AFT and XR (NABC-North American Bioproducts Corporation, GA, USA), GERT STRAND (Gert Strand AB, Sweden), SUPERSTART™ (Alltech), ANGEL™ (Angel Yeast Ltd, China) and FERMIOL™ (DSM Specialties). An ordinarily skilled artisan would understand the requirements for selection of a suitable yeast cell, and recombinant yeast cells of the present disclosure are not limited to those expressly recited herein. For example, suitable host cells and examples of recombinant cells capable of producing ethanol are described in U.S. Pat. Nos. 10,724,023, 10,334,288, US Patent Publication No. 20200270644A1, U.S. Pat. No. 11,111,482, US Patent Publication No. 20190345471A1, U.S. Pat. Nos. 11,041,218, 11,306,330, and US Patent Publication No. 20210062230A1, each of which is incorporated herein by reference in its entirety.

The recombinant cells described herein include one or more exogenous polynucleotide sequences encoding one or more exogenous polypeptides that, when expressed improve the fermentation of sucrose to lactate by the recombinant cells. The recombinant cell may alternatively or additionally include one or more genetic modifications that increases expression of a native polypeptide, wherein said increase in expression improves the fermentation of sucrose to lactate by the recombinant cell.

As used herein, “exogenous” refers to genetic material or an expression product thereof that originates from outside of the host organism. For example, the exogenous genetic material or expression product thereof can be a modified form of genetic material native to the host organism, it can be derived from another organism, it can be a modified form of a component derived from another organism, or it can be a synthetically derived component. For example, a K. lactis invertase gene is exogenous when introduced into I. orientalis.

As used herein, “native” refers to genetic material or an expression product thereof that is found, apart from individual-to-individual mutations which do not affect function or expression, within the genome of wild-type cells of the host cell.

As used herein, the terms “polypeptide” and “peptide” are used interchangeably and refer to the collective primary, secondary, tertiary, and quaternary amino acid sequence and structure necessary to give the recited macromolecule its function and properties. As used herein, “enzyme” or “biosynthetic pathway enzyme” refer to a protein that catalyzes a chemical reaction. The recitation of any particular enzyme, either independently or as part of a biosynthetic pathway is understood to include the co-factors, co-enzymes, and metals necessary for the enzyme to properly function. A summary of the amino acids and their three and one letter symbols as understood in the art is presented in Table 1. The amino acid name, three letter symbol, and one letter symbol are used interchangeably herein.

TABLE 1
Amino Acid three and one letter symbols

| Amino Acid | Three letter symbol | One letter symbol |
|---|---|---|
| Alanine | Ala | A |
| Arginine | Arg | R |
| Asparagine | Asn | N |
| Aspartic acid | Asp | D |
| Cysteine | Cys | C |
| Glutamic acid | Glu | E |
| Glutamine | Gln | Q |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Variants or sequences having substantial identity or homology with the polypeptides described herein can be utilized in the disclosed engineered cells, compositions, and methods. Such sequences can be referred to as variants or modified sequences. That is, a polypeptide sequence can be modified yet still retain the ability to exhibit the desired activity. Generally, the variant or modified sequence may include or greater than about 45%, 45%, 50%, 55%, 60%, 65%, 70%, 75%, 80%, 85%, 90%, or 95% sequence identity with the wild-type, naturally occurring polypeptide sequence, or with a variant polypeptide as described herein.

As used herein, the phrases “% sequence identity,” “% identity,” and “percent identity,” are used interchangeably and refer to the percentage of residue matches between at least two amino acid sequences or at least two nucleic acid sequences aligned using a standardized algorithm. Methods of amino acid and nucleic acid sequence alignment are well-known. Sequence alignment and generation of sequence identity include global alignments and local alignments which are carried out using computational approaches. An alignment can be performed using BLAST (National Center for Biological Information (NCBI) Basic Local Alignment Search Tool) version 2.2.31 software with default parameters. Amino acid % sequence identity between amino acid sequences can be determined using standard protein BLAST with the following default parameters: Max target sequences: 100; Short queries: Automatically adjust parameters for short input sequences; Expect threshold: 10; Word size: 6; Max matches in a query range: 0; Matrix: BLOSUM62; Gap Costs: (Existence: 11, Extension: 1); Compositional adjustments: Conditional compositional score matrix adjustment; Filter: none selected; Mask: none selected. Nucleic acid % sequence identity between nucleic acid sequences can be determined using standard nucleotide BLAST with the following default parameters: Max target sequences: 100; Short queries: Automatically adjust parameters for short input sequences; Expect threshold: 10; Word size: 28; Max matches in a query range: 0; Match/Mismatch Scores: 1, −2; Gap costs: Linear; Filter: Low complexity regions; Mask: Mask for lookup table only. A sequence having an identity score of XX % (for example, 80%) with regard to a reference sequence using the NCBI BLAST version 2.2.31 algorithm with default parameters is considered to be at least XX % identical or, equivalently, have XX % sequence identity to the reference sequence.

Polypeptide or polynucleotide sequence identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

The polypeptides disclosed herein may include “variant” polypeptides, “mutants,” and “derivatives thereof.” As used herein the term “wild-type” is a term of the art understood by skilled persons and means the typical form of a polypeptide as it occurs in nature as distinguished from variant or mutant forms. As used herein, a “variant,” “mutant,” or “derivative” refers to a polypeptide molecule having an amino acid sequence that differs from a reference protein or polypeptide molecule. A variant or mutant may have one or more insertions, deletions, or substitutions of an amino acid residue relative to a reference molecule.

The amino acid sequences of the polypeptide variants, mutants, derivatives, or fragments as contemplated herein may include conservative amino acid substitutions relative to a reference amino acid sequence. For example, a variant, mutant, derivative, or fragment polypeptide may include conservative amino acid substitutions relative to a reference molecule. “Conservative amino acid substitutions” are those substitutions that are a substitution of an amino acid for a different amino acid where the substitution is predicted to interfere least with the properties of the reference polypeptide. In other words, conservative amino acid substitutions substantially conserve the structure and the function of the reference polypeptide. Conservative amino acid substitutions generally maintain (a) the structure of the polypeptide backbone in the area of the substitution, for example, as a beta sheet or alpha helical conformation, (b) the charge and/or hydrophobicity of the molecule at the site of the substitution, and/or (c) the bulk of the side chain.

As used herein, terms “polynucleotide,” “polynucleotide sequence,” and “nucleic acid sequence,” and “nucleic acid,” are used interchangeably and refer to a sequence of nucleotides or any fragment thereof. These phrases also refer to DNA or RNA of natural or synthetic origin, which may be single-stranded or double-stranded and may represent the sense or the antisense strand. The DNA polynucleotides may be a cDNA or a genomic DNA sequence.

A polynucleotide is said to encode a polypeptide if, in its native state or when manipulated by methods known to those skilled in the art, it can be transcribed and/or translated to produce the polypeptide or a fragment thereof. The anti-sense strand of such a polynucleotide is also said to encode the sequence.

Those of skill in the art understand the degeneracy of the genetic code and that a variety of polynucleotides can encode the same polypeptide. In some aspects, the polynucleotides (i.e., polynucleotides encoding a non-heme iron-binding protein polypeptide) may be codon-optimized for expression in a particular cell including, without limitation, a plant cell, bacterial cell, fungal cell, or animal cell. While polypeptides encoded by polynucleotide sequences found in coral are disclosed herein any polynucleotide sequences may be used which encodes a desired form of the polypeptides described herein. Thus, non-naturally occurring sequences may be used. These may be desirable, for example, to enhance expression in heterologous expression systems of polypeptides or proteins. Computer programs for generating degenerate coding sequences are available and can be used for this purpose. Pencil, paper, the genetic code, and a human hand can also be used to generate degenerate coding sequences.

The recombinant cells described herein may include deletions or disruptions in one or more native genes. The phase “deletion or disruption” refers to the status of a native gene in the recombinant cell that has either a completely eliminated coding region (deletion) or a modification of the gene, its promoter, or its terminator (such as be a deletion, insertion, or mutation) so that the gene no longer produces an active expression product, produces severely reduced quantities of the expression product (e.g., at least a 75% reduction or at least a 90% reduction) or produces an expression product with severely reduced activity (e.g., at least 75% reduced or at least 90% reduced). The deletion or disruption can be achieved by genetic engineering methods, forced evolution, mutagenesis, and/or selection and screening. The native gene to be deleted or disrupted may be replaced with an exogenous nucleic acid of interest for the expression of an exogenous gene product (e.g., polypeptide, enzyme, and the like).

The recombinant cell described herein may have a deletion or disruption of one or more native genes encoding an enzyme involved in the synthesis of glycerol. Deletion or disruption of one or more of these glycerol biosynthetic pathway enzymes decreases the ability of the cell to product glycerol, thereby increasing fermentation production of ethanol.

The recombinant cells described herein may include a deletion or disruption of a native glycerol-3-phosphate phosphatase (GPP) gene. The native GPP gene(s) encode an enzyme that catalyzes the hydrolysis of glycerol-3-phosphate into glycerol. When the host cell contains multiple GPP genes, it is preferred to delete or disrupt at least one of them and more preferred to disrupt all of them to more completely eliminate the host cell's ability to product glycerol. In S. cerevisiae, there are two GPP paralogs, referred to as Gpp1p (SEQ ID NO:120), encoded by the GPP1 gene, and Gpp2p (SEQ ID NO:121), encoded by the GPP2 gene. When the recombinant cell is a S. cerevisiae cell, the cell may include a deletion or disruption of a GPP gene encoding an amino acid sequence at least 60%, at least 65%, at least 70%, at least 75%, at least 80%, at least 85%, at least 90%, at least 95%, at least 98%, at least 99%, or 100% identical to at least one of SEQ ID NOs: 120 and 121. Methods for the deletion or disruption of the GPP genes of S. cerevisiae are known and described in the art and are exemplified herein.

The recombinant cells described herein may include a deletion or disruption of a native glycerol-3-phosphate dehydrogenase (GPD) gene. Deletion or disruption of a native GPD gene improves acetate consumption by providing the cell with a greater pool of reducing equivalents to assist in the oxido-reduction of acetate to ethanol. When the host cell contains multiple GPD genes, it is preferred to delete or disrupt at least one of them and more preferred to disrupt all of them. In S. cerevisiae, there are two glycerol-3-phosphate dehydrogenases, referred to as Gpd1p, encoded by GPD1, and Gpd2p, encoded by GPD2.

The recombinant cells described herein may include one or more genetic modifications in which an exogenous nucleic acid is integrated into the genome of the host cell. One of skill in the art know how to select suitable loci in a yest genome for integration of the exogenous nucleic acid. For example, in an S. cerevisiae host cells, suitable interaction loci may include, but are not limited to, the GPP1 loci (defined as the loci flanked by SEQ ID NO:136 and SEQ ID NO:137), the DLD1 loci (defined as the loci flanked by SEQ ID NO:138 and SEQ ID NO: 139), and the GPD1 loci (defined as the loci flanked by SEQ ID NO:140 and SEQ ID NO:141). Other suitable integration loci may be determined one of skill in the art. Furthermore, one of skill in the art would recognize how to use sequences to design primers to verify correct gene integration at the chosen locus.

The recombinant cells described herein are capable of producing ethanol and include an exogenous polynucleotide sequence encoding a glyceraldehyde-3-phosphate dehydrogenase (gapN) enzyme. The gapN enzyme may be any suitable enzyme with glyceraldehyde-3-phosphate dehydrogenase activity. The exogenous polynucleotide sequence may be an exogenous glyceraldehyde-3-phosphate dehydrogenase (gapN) gene.

A “glyceraldehyde-3-phosphate dehydrogenase gene” and “gapN gene” are used interchangeably herein and refer to any gene or polynucleotide that encodes a polypeptide with glyceraldehyde-3-phosphate dehydrogenase activity. As used herein “glyceraldehyde-3-phosphate dehydrogenase activity” refers to the ability to catalyze the conversion of D-glyceraldehyde 3-phosphate and NADP⁺ to 3-phospho-D-glycerate and NADPH. The gapN enzyme can be from any suitable source organism or may be synthetic. Suitable gapN enzymes may include, but are not limited to, enzymes categorized under Enzyme Commission (EC) number 1.2.1.9, also known in the art as “NADP-dependent non-phosphorylating glyceraldehyde-3-phosphate dehydrogenase.” Suitable gapN enzymes may be the gapN enzymes from Streptococcus pyogenes, Pseudomonas fluorescens, Brevibacillus laterosporus, Arabidopsis thaliana, Chryseobacterium gleum, Streptococcus mutans, Streptococcus henryi, Lactobacillus delbrueckii, Bacillus cereus, and the like. The gapN gene may encode an amino acid at least 60%, at least 70%, at least 80%, at least 85%, at least 90%, at least 95%, at least 97%, at least 99%, or 100% identical to at least one of SEQ ID NOs: 28, 32, 48, 52, 64, 68, 80, 92, and 96. The gapN gene may encode an amino acid at least 60%, at least 70%, at least 80%, at least 85%, at least 90%, at least 95%, at least 97%, at least 99%, or 100% identical to at least one of SEQ ID NOs: 28, 32, 48, 52, and 64. The gapN gene may encode an amino acid at least 60%, at least 70%, at least 80%, at least 85%, at least 90%, at least 95%, at least 97%, at least 99%, or 100% identical to at least one of SEQ ID NOs: 28, 32, 52, and 64.

The recombinant cell may comprise an exogenous polynucleotide that is or may be derived from a Streptococcus pyogenes gene encoding the amino acid sequences of SEQ ID NO: 28. The exogenous polynucleotide may encode an amino acid sequence with at least 60%, at least 70%, at least 80%, at least 85%, at least 90%, at least 95%, at least 97%, at least 99%, or 100% identical to SEQ ID NO:28.

The recombinant cell may comprise an exogenous polynucleotide that is or may be derived from a Pseudomonas fluorescens gene encoding the amino acid sequences of SEQ ID NO: 32. The exogenous polynucleotide may encode an amino acid sequence with at least 60%, at least 70%, at least 80%, at least 85%, at least 90%, at least 95%, at least 97%, at least 99%, or 100% identical to SEQ ID NO:32.

The recombinant cell may comprise an exogenous polynucleotide that is or may be derived from a Brevibacillus laterosporus gene encoding the amino acid sequences of SEQ ID NO: 48. The exogenous polynucleotide may encode an amino acid sequence with at least 60%, at least 70%, at least 80%, at least 85%, at least 90%, at least 95%, at least 97%, at least 99%, or 100% identical to SEQ ID NO:48.

The recombinant cell may comprise an exogenous polynucleotide that is or may be derived from a Arabidopsis thaliana gene encoding the amino acid sequences of SEQ ID NO:52. The exogenous polynucleotide may encode an amino acid sequence with at least 60%, at least 70%, at least 80%, at least 85%, at least 90%, at least 95%, at least 97%, at least 99%, or 100% identical to SEQ ID NO:52.

The recombinant cell may comprise an exogenous polynucleotide that is or may be derived from a Chryseobacterium gleum gene encoding the amino acid sequences of SEQ ID NO: 64. The exogenous polynucleotide may encode an amino acid sequence with at least 60%, at least 70%, at least 80%, at least 85%, at least 90%, at least 95%, at least 97%, at least 99%, or 100% identical to SEQ ID NO:64.

The recombinant cell may comprise an exogenous polynucleotide that is or may be derived from a Streptococcus mutans gene encoding the amino acid sequences of SEQ ID NO:68. The exogenous polynucleotide may encode an amino acid sequence with at least 60%, at least 70%, at least 80%, at least 85%, at least 90%, at least 95%, at least 97%, at least 99%, or 100% identical to SEQ ID NO:68.

The recombinant cell may comprise an exogenous polynucleotide that is or may be derived from a Streptococcus henryi gene encoding the amino acid sequences of SEQ ID NO:80. The exogenous polynucleotide may encode an amino acid sequence with at least 60%, at least 70%, at least 80%, at least 85%, at least 90%, at least 95%, at least 97%, at least 99%, or 100% identical to SEQ ID NO:80.

The recombinant cell may comprise an exogenous polynucleotide that is or may be derived from a Lactobacillus delbrueckii gene encoding the amino acid sequences of SEQ ID NO: 92. The exogenous polynucleotide may encode an amino acid sequence with at least 60%, at least 70%, at least 80%, at least 85%, at least 90%, at least 95%, at least 97%, at least 99%, or 100% identical to SEQ ID NO:92.

The recombinant cell may comprise an exogenous polynucleotide that is or may be derived from a Bacillus cereus gene encoding the amino acid sequences of SEQ ID NO:96. The exogenous polynucleotide may encode an amino acid sequence with at least 60%, at least 70%, at least 80%, at least 85%, at least 90%, at least 95%, at least 97%, at least 99%, or 100% identical to SEQ ID NO:96.

The recombinant cells described herein are capable of producing ethanol, include an exogenous polynucleotide sequence encoding a gapN enzyme, and may additionally include an exogenous polynucleotide sequence encoding an alcohol dehydrogenase (ADH) enzyme. The ADH enzyme may be any suitable enzyme with NADP-dependent alcohol dehydrogenase activity. The exogenous polynucleotide sequence may be an exogenous alcohol dehydrogenase (ADH) gene.

An “alcohol dehydrogenase gene” and “ADH gene” are used interchangeably herein and refer to any gene or polynucleotide that encodes a polypeptide with alcohol dehydrogenase activity. As used herein, “alcohol dehydrogenase activity” refers to the ability to catalyze the conversion of acetaldehyde and NADH or NADPH to ethanol and NAD⁺ or NADP⁺. As used herein, NADP-dependent alcohol dehydrogenase activity” refers to the ability to catalyze the conversion of acetaldehyde and NADPH to ethanol and NADP⁺. The ADH enzyme may be derived from any suitable source or may be synthetic. Suitable ADH enzymes may be the ADH enzymes from Rhodotorula toruloides, Candida maltosa, and the like. The ADH gene may encode an amino acid at least 60%, at least 70%, at least 80%, at least 85%, at least 90%, at least 95%, at least 97%, at least 99%, or 100% identical to at least one of SEQ ID NOs: 98 and 100.

The recombinant cell may comprise an exogenous polynucleotide that is or may be derived from a Candida maltosa gene encoding the amino acid sequences of SEQ ID NO:98. The exogenous polynucleotide may encode an amino acid sequence with at least 60%, at least 70%, at least 80%, at least 85%, at least 90%, at least 95%, at least 97%, at least 99%, or 100% identical to SEQ ID NO:98.

The recombinant cell may comprise an exogenous polynucleotide that is or may be derived from a Rhodotorula toruloides gene encoding the amino acid sequences of SEQ ID NO: 100. The exogenous polynucleotide may encode an amino acid sequence with at least 60%, at least 70%, at least 80%, at least 85%, at least 90%, at least 95%, at least 97%, at least 99%, or 100% identical to SEQ ID NO:100.

The recombinant cells described herein are capable of producing ethanol, include an exogenous polynucleotide sequence encoding a gapN enzyme, may optionally include an exogenous polynucleotide sequence encoding an ADH enzyme, and may additionally include an exogenous polynucleotide sequence encoding a glucoamylase (GA) enzyme. The GA enzyme may be any suitable enzyme with glucoamylase activity. The exogenous polynucleotide sequence may be an exogenous glucoamylase (GA) gene.

A “glucoamylase gene” and “GA gene” are used interchangeably herein and refer to any gene or polynucleotide that encodes a polypeptide with glucoamylase activity. As used herein, “glucoamylase activity” refers to the ability to catalyze the hydrolysis of the terminal 1,4-linked alpha-D-glucose residue from the non-reducing end of an amylose chain to release free glucose. The GA enzyme can be from any suitable source organism or may be synthetic. Suitable glucoamylase enzymes may include, but are not limited to, enzymes of EC 3.2.1.3. Suitable GA enzymes may be the GA enzymes from Saccharomycopsis fibuligera, Rhizopus delemar, Rhizopus microspores, Rhizopus oryzae, and the like. The GA gene may encode an amino acid at least 60%, at least 70%, at least 80%, at least 85%, at least 90%, at least 95%, at least 97%, at least 99%, or 100% identical to at least one of SEQ ID NOs: 1, 122, 123, and 124. The GA gene may encode an amino acid at least 60%, at least 70%, at least 80%, at least 85%, at least 90%, at least 95%, at least 97%, at least 99%, or 100% identical to at least one of SEQ ID NOs: 1, 123, and 124. Additional GA genes and GA enzyme sequences are known and described in the art, such as GA genes encoding GA enzymes with modified leader/signal sequences. See, for example, U.S. Pat. Nos. 10,364,421, 10,724,023, US Patent Publication No. 20190345471A1, and U.S. Pat. No. 11,306,330, each of which is incorporated by reference herein in its entirety.

The recombinant cell may comprise an exogenous polynucleotide that is or may be derived from a Rhizopus microspores gene encoding the amino acid sequences of SEQ ID NO:1. The exogenous polynucleotide may encode an amino acid sequence with at least 60%, at least 70%, at least 80%, at least 85%, at least 90%, at least 95%, at least 97%, at least 99%, or 100% identical to SEQ ID NO:124.

The recombinant cell may comprise an exogenous polynucleotide that is or may be derived from a Saccharomycopsis fibuligera gene encoding the amino acid sequences of SEQ ID NO: 122. The exogenous polynucleotide may encode an amino acid sequence with at least 60%, at least 70%, at least 80%, at least 85%, at least 90%, at least 95%, at least 97%, at least 99%, or 100% identical to SEQ ID NO:122.

The recombinant cell may comprise an exogenous polynucleotide that is or may be derived from a Rhizopus delemar gene encoding the amino acid sequences of SEQ ID NO:123. The exogenous polynucleotide may encode an amino acid sequence with at least 60%, at least 70%, at least 80%, at least 85%, at least 90%, at least 95%, at least 97%, at least 99%, or 100% identical to SEQ ID NO:123.

The recombinant cell may comprise an exogenous polynucleotide that is or may be derived from a Rhizopus oryzae gene encoding the amino acid sequences of SEQ ID NO:124. The exogenous polynucleotide may encode an amino acid sequence with at least 60%, at least 70%, at least 80%, at least 85%, at least 90%, at least 95%, at least 97%, at least 99%, or 100% identical to SEQ ID NO: 124.

The exogenous nucleic acids in the recombinant cells described herein may be under the control of a promoter. For example, the exogenous nucleic acid may be operably linked to a heterologous or artificial promoter. Suitable promoters are known and described in the art. Promoters may include, but are not limited to, pyruvate decarboxylase (PDC1), glyceraldehyde-3-phosphate dehydrogenase (GAPDH) (TDH3 herein; annotated in EC 1.2.1.12; SEQ ID NO: 125), translational elongation factor 1 (TEF1; SEQ ID NO:128), URA3 (SEQ ID NO:126), S-adenosyl methionine transferase 2 (SAM2; SEQ ID NO:129), alcohol dehydrogenase 1 (ADH1; SEQ ID NO:130) and 3-phosphoglycerate kinase (PGK1; SEQ ID NO:127).

The exogenous nucleic acids in the recombinant cells described herein may be under the control of a terminator. For example, the exogenous nucleic acid may be operably linked to a heterologous or artificial terminator. Suitable terminators are known and described in the art. Terminators may include, but are not limited to, iso-1-cytochrome c (CYC1; SEQ ID NO:131), URA3 (SEQ ID NO:132), PDC, ADHI (SEQ ID NO:134), TEF1 (SEQ ID NO:135), and ScGAL10 (SEQ ID NO:133).

A promoter or terminator is “operably linked” to a given polynucleotide (e.g., a gene) if its position in the genome or expression cassette relative to said polynucleotide is such that the promoter or terminator, as the case may be, performs its transcriptional control function.

The polypeptides described herein may be provided as part of a construct. As used herein, the term “construct” refers to recombinant polynucleotides including, without limitation, DNA and RNA, which may be single-stranded or double-stranded and may represent the sense or the antisense strand. Recombinant polynucleotides are polynucleotides formed by laboratory methods that include polynucleotide sequences derived from at least two different natural sources or they may be synthetic. Constructs thus may include new modifications to endogenous genes introduced by, for example, genome editing technologies. Constructs may also include recombinant polynucleotides created using, for example, recombinant DNA methodologies. The construct may be a vector including a promoter operably linked to the polynucleotide encoding the thermolabile non-heme iron-binding polypeptide. As used herein, the term “vector” refers to a polynucleotide capable of transporting another polynucleotide to which it has been linked. The vector may be a plasmid, which refers to a circular double-stranded DNA loop into which additional DNA segments may be integrated.

The disclosure also provides fermentation methods for the production of ethanol using the recombinant cells described herein. The fermentation methods include the step of fermenting a substrate using the genetically engineered yeasts described herein to product ethanol. The fermentation method can include additional steps, as would be understood by a person skilled in the art. Non-limiting examples of additional process steps include maintaining the temperature of the fermentation broth within a predetermined range, adjusting the pH during fermentation, and isolating the ethanol from the fermentation broth.

The fermentation substrate can comprise a starch. Starch can be obtained from a natural source, such as a plant source. Starch can also be obtained from a feedstock with high starch or sugar content, including, but not limited to corn, sweet sorghum, fruits, sweet potato, rice, barley, sugar cane, sugar beets, wheat, cassava, potato, tapioca, arrowroot, peas, or sago. The fermentation substrate may be from lignocellulosic biomass such as wood, straw, grasses, or algal biomass, such as microalgae and macroalgae. The fermentation substrate may include cellulosic or lignocellulosic biomass. The fermentation substrate may be from grasses, trees, or agricultural and forestry residues, such as corn cobs and stalks, rice straw, sawdust, and wood chips. The fermentation substrate can also comprise a sugar, such as glucose (dextrose) or sucrose. The fermentation substrate may comprise a dry grind ethanol feedstock, such as corn mash. The fermentation substrate can comprise a liquefied corn mash (LCM). The fermentation substrate may comprise a corn wet mill feedstock, such as Light Steep Water/Liquifact (LSW/LQ).

Media for fermentation of the engineered yeast described herein can be supplemented with various components. For example, media for fermentation of the engineered yeast described herein can be supplemented with a glucoamylase, e.g., the glucoamylase Spirizyme™ (Novozymes, Bagsvaerd, Denmark).

The fermentation process can be run under various conditions. The fermentation temperature, i.e., the temperature of the fermentation broth during processing, may be ambient temperature. Alternatively, or additionally, the fermentation temperature may be maintained within a predetermined range. For example, the fermentation temperature can be maintained in the range of 25° C. to 40° C., 27° C. to 38° C., or 30° C. to 35° C. The fermentation temperature may be maintained at a temperature of, e.g., 25, 26, 27, 28, 29, 30, 31, 32, 33, 34, 35, 36, 37, 38, 39, 40° C., or any value in between. However, a skilled artisan will recognize that the fermentation temperature is not limited to any specific range recited herein and may be modified as appropriate.

The pH of a culture medium described herein may be controlled for optimal ethanol production. The pH of the culture or a fermentation mixture of an engineered cell described herein may be in the range of between 4.0 and 6.0. The pH may be maintained for at least part of the incubation at 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, or 6.0. The pH may be maintained at a range between 5.0 and 5.5.

The engineered yeast may be cultured for approximately 24-72 hours. For example, the engineered yeast may be cultured for approximately 12, 18, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 78, 80, 90, 96 hours, or more than 96 hours. The engineered yeast described herein may be cultured for approximately 48 to 72 hours. A culture (fermentation) time of about 48 hours is a representative time for commercial-scale ethanol fermentation processes. Accordingly, a 48 hour time point can be used to compare the fermentation performance of different yeast strains.

Reaction parameters can be measured or adjusted during the production of ethanol. Non-limiting examples of reaction parameters include biological parameters (e.g., growth rate, cell size, cell number, cell density, cell type, or cell state, etc.), chemical parameters (e.g., pH, redox-potential, concentration of reaction substrate and/or product, concentration of dissolved gases, such as oxygen concentration and CO2 concentration, nutrient concentrations, metabolite concentrations, ethanol concentration, fermentation substrate concentration, concentration of an oligopeptide, concentration of an amino acid, concentration of a vitamin, concentration of a hormone, concentration of an additive, serum concentration, ionic strength, concentration of an ion, relative humidity, molarity, osmolarity, concentration of other chemicals, for example buffering agents, adjuvants, or reaction by-products), physical/mechanical parameters (e.g., density, conductivity, degree of agitation, pressure, and flow rate, shear stress, shear rate, viscosity, color, turbidity, light absorption, mixing rate, conversion rate, as well as thermodynamic parameters, such as temperature, light intensity/quality, etc.). Sensors to measure the parameters described herein are well known to one of ordinary skill in the art.

The fermentation process can be associated with various characteristics, such as, but not limited to, fermentation production rate, pathway fermentation yield, final titer, and peak fermentation rate. These characteristics can be affected by the selection of the yeast and/or genetic modification of the yeast used in the fermentation process. These characteristics can be affected by adjusting the fermentation process conditions. These characteristics can be adjusted via a combination of yeast selection or modification and the selection of fermentation process conditions.

The ethanol production rate of the process may be at least 1.0, at least 1.5, or at least 2.0, at least 2.5, at least 3.0, or at least 3.5 g L⁻¹ h⁻¹. The final ethanol titer of the process may beat least 60 g/L, at least 80, at least 100, or at least 120 g/L. The final glycerol titer of the process may be less than 10 g/L, less than 8 g/L, less than 6 g/L, less than 4 g/L, or less than 3 g/L.

### EXAMPLES

The invention is further described in detail by reference to the following experimental examples. These examples are provided for purposes of illustration only and are not intended to be limiting unless otherwise specified. Thus, the invention should in no way be construed as being limited to the following examples, but rather should be construed to encompass any and all variations which become evident as a result of the teaching provided herein.

### Example 1: gapN Biodiversity

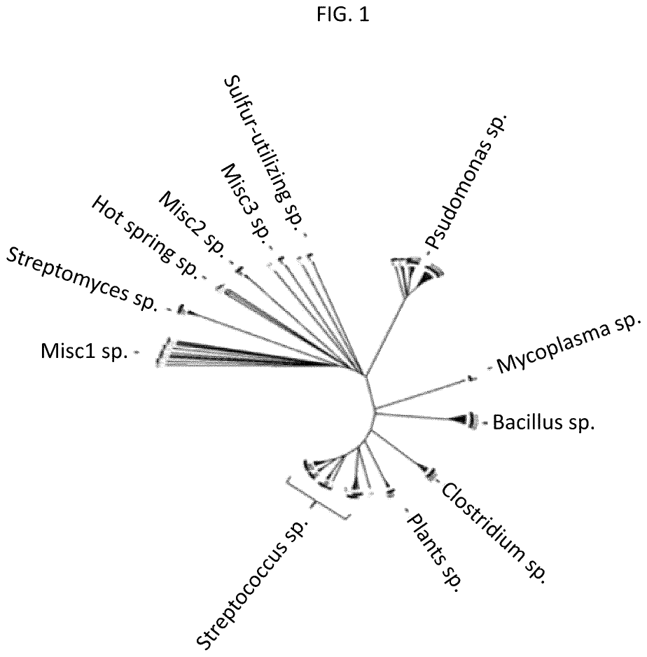

Previous work demonstrated the effectiveness of the B. cereus gapN for reduction of glycerol by-product formation and increasing ethanol titers in S. cerevisiae (See, for example, US Patent Application Publication No. US 2021/006230, which is incorporated herein by reference in its entirety). However, there is still room for further improvement in reducing glycerol and increasing ethanol production. The biodiversity of gapN was surveyed, revealing fewer than 1,00 annotated gapN genes, primarily in the Streptococcus (230 gapN genes), Pseudomonas (94), Bacillus (95), and Clostridium genera (FIG. 1). Based on this analysis, 24 gapN genes were selected for further analysis.

### Example 2: Genetically Modified Yeast Strains-gapN

### Strain 1-1

Strain 1-17 described by Poynter et al. (US Patent Application Publication No. US 2021/006230 published Mar. 4, 2021, incorporated herein by reference in its entirety) is a Saccharomyces cerevisiae host strain that is ura3 positive (ura3+) and amdS positive (amdS+) and in which both alleles of the cytosine deaminase (FCY1) gene are knocked out and replaced with an expression cassette for the Rhizopus microsporus glucoamylase of SEQ ID NO: 1 with a TDH3 promoter and a CYC1 terminator. Herein, Strain 1-1 refers to Strain 1-17 of Poynter et al. US Application Publication No. US 2021/006230.

### Strain 1-2

Strain 1-18 described by Poynter et al. (US Patent Application Publication No. US 2021/006230 published Mar. 4, 2021, incorporated herein by reference in its entirety) is a Saccharomyces cerevisiae host strain that is ura3 negative (ura3-) and amdS negative (amdS-) and in which both alleles of the cytosine deaminase (FCY1) gene are knocked out and replaced with an expression cassette for the Rhizopus microsporus glucoamylase of SEQ ID NO: 1 with a TDH3 promoter and a CYC1 terminator. Herein, Strain 1-2 refers to Strain 1-18 of Poynter et al. US Application Publication No. US 2021/006230.

### Strain 1-3

Strain 1-25 described by Poynter et al. (US Patent Application Publication No. US 2021/006230 published Mar. 4, 2021, incorporated herein by reference in its entirety) is a Saccharomyces cerevisiae host strain that is ura3+ and amdS+, in which both alleles of the FCY1 gene are knocked out and replaced with an expression cassette for the Rhizopus microsporus glucoamylase of SEQ ID NO: 1 with a TDH3 promoter and a CYC1 terminator and in which both alleles of the GPP1 gene are replaces with an expression cassette for the Bacillus cereus glyceraldehyde-3-phosphate dehydrogenase (gapN) of SEQ ID NO: 96. Herein, Strain 1-3 refers to Strain 1-25 of Poynter et al. US Application Publication No. US 2021/006230.

### Strains 1-4 Through 1-27

Strain 1-2 was transformed with the “Upstream Fragment” and “Downstream Fragment” as indicated in Table 2 to produce strains 1-4 through 1-27. Each “Upstream Fragment” contained i) a 5′ GPP1 flanking sequence; ii) a ScTDH3 promoter; iii) a gene encoding the indicated gapN; iv) an ScCYC1 terminator; v) a loxP site; vi) a ScURA3 promoter; and vii) a 5′ portion of the ScURA open reading frame. Each “Downstream Fragment” contained i) a 3′ GPP1 flanking sequence; ii) a ScTDH3 promoter; iii) a gene encoding the indicated gapN; iv) a ScPGK1 terminator, v) a loxP site; vi) a ScURA3 terminator; and vii) a 3′ portion of the ScURA open reading frame. Transformants were selected on synthetic dropout media lacking uracil (ScD-Ura) plates. Resulting transformants were streaked for single colony isolation on ScD-Ura plates. A single colony was selected. Correct integration of the indicated gene encoding the gapN was verified by PCR and the PCR verified isolate was designated with the strain identifier outlined in Table 2. Resulting strains 1~4 through 1-20, 1-23, 1-24, 1-26 and 1-27 included two copies of the indicated gapN expression cassette in tandem on a single allele of the GPP1 gene. Resulting strains 1-21, 1-22, and 1-25 included one copy of the indicated gapN expression cassette and one copy of the B. cereus gapN expression cassette in tandem on a single allele of the GPP1 gene.

TABLE 2

| | | Upstream | Downstream | gapN Homolog | | |
|---|---|---|---|---|---|---|
| | | Fragment | Fragment | | Encoded | gapN |
| | Parent | SEQ | SEQ | Encoded gapN | gapN SEQ | nucleotide |
| Strain | Strain | ID NO: | ID NO: | source organism | ID NO: | SEQ ID NO: |
| 1-4 | 1-2 | 2 | 3 | Roseisalinus antarcticus | 4 | 5 |
| 1-5 | 1-2 | 6 | 7 | Oceanibacterium | 8 | 9 |
| | | | | hippocampi | | |
| 1-6 | 1-2 | 10 | 11 | Bacillus thuringiensis | 12 | 13 |
| 1-7 | 1-2 | 14 | 15 | Defluviimonas aquaemixtae | 16 | 17 |
| 1-8 | 1-2 | 18 | 19 | Planctomyces sp | 20 | 21 |
| 1-9 | 1-2 | 22 | 23 | Bacillus abyssalis | 24 | 25 |
| 1-10 | 1-2 | 26 | 27 | Streptococcus pyogenes | 28 | 29 |
| 1-11 | 1-2 | 30 | 31 | Pseudomonas fluorescens | 32 | 33 |
| 1-12 | 1-2 | 34 | 35 | Bacillus megaterium | 36 | 37 |
| 1-13 | 1-2 | 38 | 39 | Clostridium | 40 | 41 |
| | | | | saccharoperbutylacetonicum | | |
| 1-14 | 1-2 | 42 | 43 | Rhodobacteraceae bacterium | 44 | 45 |
| 1-15 | 1-2 | 46 | 47 | Brevibacillus laterosporus | 48 | 49 |
| 1-16 | 1-2 | 50 | 51 | Arabidopsis thaliana | 52 | 53 |
| 1-17 | 1-2 | 54 | 55 | Thermococcus sibiricus | 56 | 57 |
| 1-18 | 1-2 | 58 | 59 | Lactobacillus kisonensis | 60 | 61 |
| 1-19 | 1-2 | 62 | 63 | Chryseobacterium gleum | 64 | 65 |
| 1-20 | 1-2 | 66 | 67 | Streptococcus mutans | 68 | 69 |
| 1-21* | 1-2 | 70 | 95 | Exiguobacterium flavidum | 72 | 73 |
| 1-22* | 1-2 | 74 | 95 | Flavobacterium collinsii | 76 | 77 |
| 1-23 | 1-2 | 78 | 79 | Streptococcus henryi | 80 | 81 |
| 1-24 | 1-2 | 82 | 83 | Bacillus beveridgei | 84 | 85 |
| 1-25* | 1-2 | 86 | 95 | Glycine soja | 88 | 89 |
| 1-26 | 1-2 | 90 | 91 | Lactobacillus delbrueckii | 92 | 93 |
| 1-27 | 1-2 | 94 | 95 | Bacillus cereus | 96 | 97 |

*These strains include, in tandem on a single allele of the GPP1 gene, an expression cassette for one copy of the indicated gapN and one copy of the B. cereus gapN of SEQ ID NO: 96.

### Example 3: Deep Well Assays

Strains 1-1 (negative control), 1-3 (positive control) and 1-4 through 1-27 were run in 96-deep well plates to assay ethanol and glycerol production.

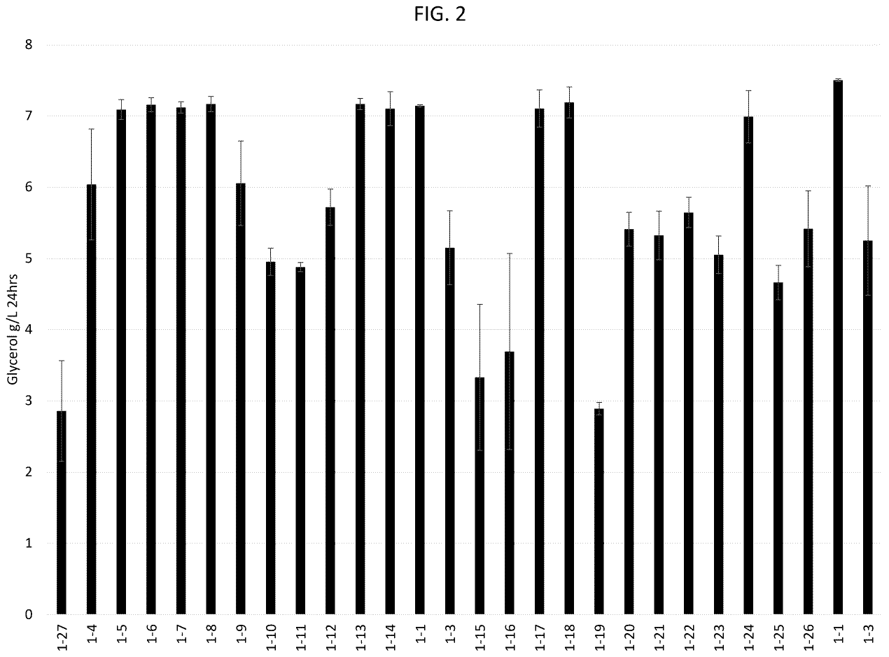

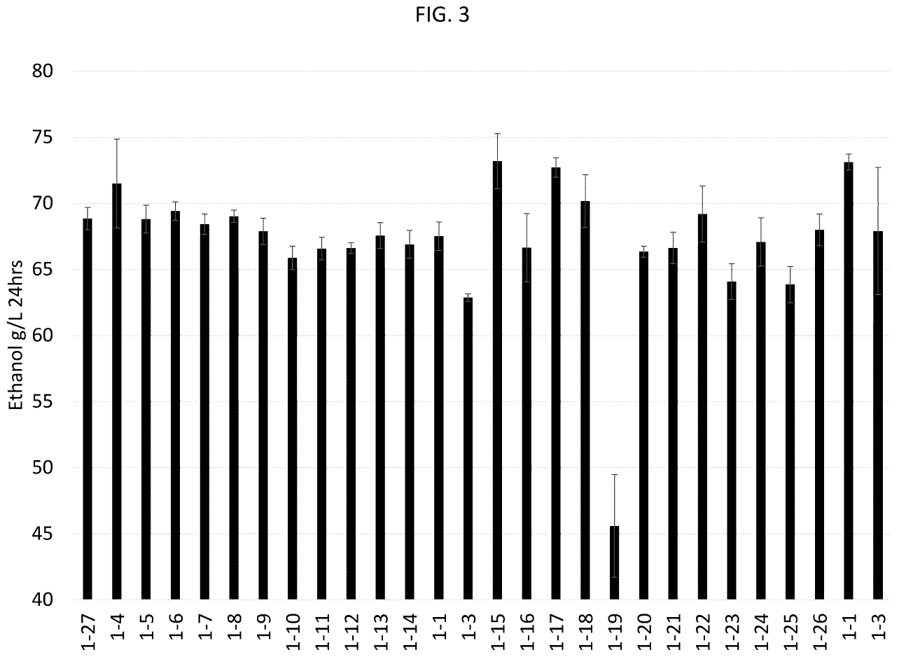

Strains were struck to a ScD-ura plate and incubated at 30° C. until single colonies were visible (2-3 days). Cells from the ScD-ura plate were scraped into 20 g/L YPD media and grown overnight in 96-deep well plates as 30° C. and 800 rpm. Immediately prior to inoculating, 2 mL shake flask medium containing sterilized canola oil was added to each well of a 96-deep well plate. The shake flask medium consisted of 725 g partially hydrolyzed corn starch, 150 g filtered sterilized (0.2 μm) light steep water, 10 g water, 25 g glucose, and 1 g urea. Strains were incubated at 35° C. with shaking in an orbital shake at 250 rpm for about 70 hours. Samples were taken and analyzed for ethanol and glycerol concentrations by HPLC. The average over 6 wells per strain is reported in Table 3 and FIGS. 2 and 3.

TABLE 3

| | Glycerol | | Ethanol | |
|---|---|---|---|---|
| Strain | Average | Standard Deviation | Average | Standard Deviation |

Plate 1

| | | | | |
|---|---|---|---|---|
| 1-1 | 7.140 | 0.016 | 67.504 | 1.069 |
| 1-3 | 5.149 | 0.519 | 62.858 | 0.274 |
| 1-4 | 6.037 | 0.778 | 71.491 | 3.357 |
| 1-5 | 7.088 | 0.140 | 68.796 | 1.053 |
| 1-6 | 7.155 | 0.099 | 69.409 | 0.693 |
| 1-7 | 7.118 | 0.079 | 68.413 | 0.769 |
| 1-8 | 7.165 | 0.107 | 69.010 | 0.469 |
| 1-9 | 6.054 | 0.593 | 67.877 | 0.996 |
| 1-10 | 4.951 | 0.192 | 65.863 | 0.882 |
| 1-11 | 4.877 | 0.065 | 66.563 | 0.854 |
| 1-12 | 5.717 | 0.254 | 66.602 | 0.399 |
| 1-13 | 7.167 | 0.079 | 67.551 | 0.977 |
| 1-14 | 7.100 | 0.239 | 66.886 | 1.054 |
| 1-27 | 2.858 | 0.707 | 68.833 | 0.842 |

Plate 2

| | | | | |
|---|---|---|---|---|
| 1-1 | 7.500 | 0.019 | 73.107 | 0.605 |
| 1-3 | 5.250 | 0.768 | 67.892 | 4.809 |
| 1-15 | 3.329 | 1.025 | 73.182 | 2.079 |
| 1-16 | 3.692 | 1.376 | 66.635 | 2.572 |
| 1-17 | 7.104 | 0.261 | 72.704 | 0.738 |
| 1-18 | 7.188 | 0.217 | 70.159 | 1.998 |
| 1-19 | 2.891 | 0.085 | 45.585 | 3.883 |
| 1-20 | 5.409 | 0.236 | 66.338 | 0.420 |
| 1-21 | 5.322 | 0.342 | 66.617 | 1.195 |
| 1-22 | 5.644 | 0.213 | 69.179 | 2.119 |
| 1-23 | 5.050 | 0.264 | 64.071 | 1.343 |
| 1-24 | 6.988 | 0.367 | 67.070 | 1.824 |
| 1-25 | 4.660 | 0.241 | 63.835 | 1.372 |
| 1-26 | 5.414 | 0.533 | 67.979 | 1.201 |

Several strains showed similar glycerol levels to the control strain 1-1, indicating that the introduced gapN homologs did not reduce glycerol production in an ethanol fermentation reaction. However, several strains did show reduced levels of glycerol production relative to the control strain 1-1, with the reduced levels being equivalate or better than the glycerol levels of the comparative strain 1-3 expressing the B. cereus gapN. In particular strains 1-15, 1-16, and 1-19 showed improved glycerol reduction compared to both strains 1-1 and 1-3. Strains 1-10, 1-11, 1-20, 1-23, and 1-26 showed improved glycerol reduction relative to strain 1-1 but at an equivalent level to strain 1-3. Comparing strains 1-27 and 1-3, both have two copies of the B. cereus gapN expression set, however one has one copy at each of the two GPP1 alleles (1-3) while the other has both copes in tandem on a single allele (1-27). Reduced glycerol production in both 1-3 and 1-27 relative to 1-1 demonstrates that the location of gapN expression cassettes can be variable and the beneficial reduction in glycerol production can be seen even when one GPP1 allele is present.

### Example 4: ADH Biodiversity

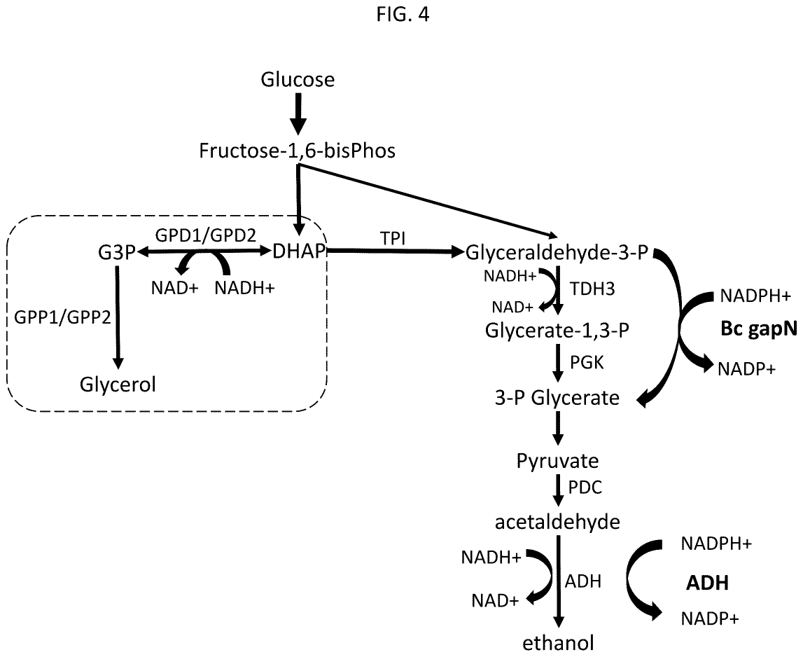

The biodiversity of alcohol dehydrogenase (ADH) enzymes was surveyed and 41 were selected for testing. Genes encoding the selected ADH enzymes were cloned into Saccharomyces cerevisiae, biomass was grown, the resulting cells were lysed, and cell free extracts were assayed using ethanol and NAD/NADP as the substrate. While this reaction is the opposite of the desired in vivo activity of the ADH, it is a suitable characterization of the NAD vs NADP preference of the enzyme. Without wishing to be bound by any particular theory, embodiment, or mode of action, it is believed that engineering a yeast cell with an ADH enzyme that is cofactor matched to a given gapN will improve both glycerol reduction and ethanol production in the cell due to the redox balancing (See FIG. 4).

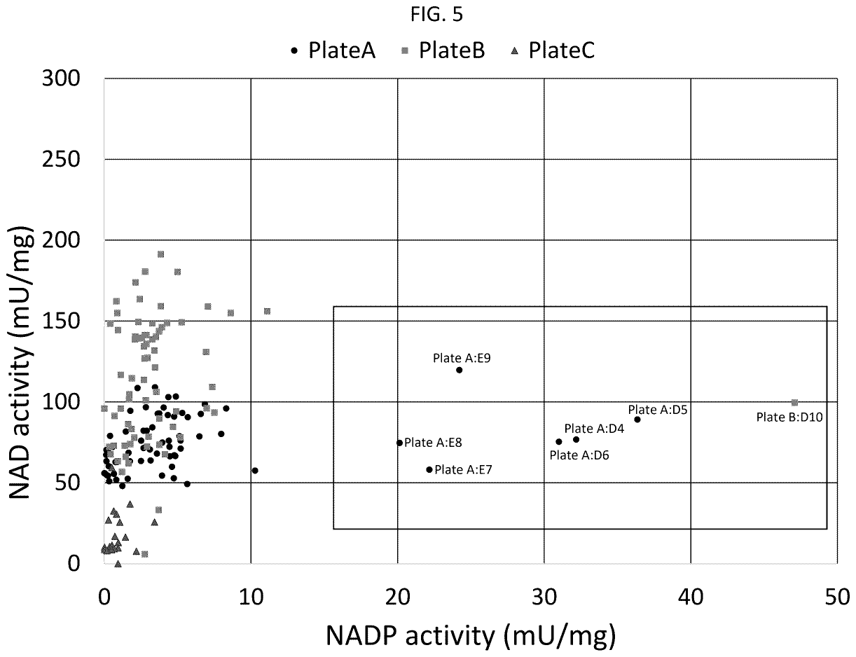

As shown in the FIG. 5, seven wells of the assay (shown in the box) showed a higher level of activity when using NADP as the cofactor. Additional information on these top hits is provided in Table 4. Based on these results the ADH enzymes from Candida maltosa (SEQ ID NOs: 98 and 99) and Rhodotorula toruloides (SEQ ID NOs: 100 and 101) were chosen for further in vivo testing.

TABLE 4

| | | NADP activity | NAD activity | Ratio |
|---|---|---|---|---|
| Enzyme | Notes | (mU/mg) | (mU/mg) | (NADP/NAD) |
| R. toruloides | First triplicate | 22.2 ± 2.0 | 84.2 ± 31.9 | 0.284 |
| SEQ ID NO: 100 | well average | | | |
| R. toruloides | Second triplicate | 32.2 ± 2.8 | 80.5 ± 7.6 | 0.413 |
| SEQ ID NO: 100 | well average | | | |
| C. maltosa | Triplicate well | 20.5 ± 23.0 | 96.3 ± 3.1 | 0.21 |
| SEQ ID NO: 98 | average | | | |
| C. maltosa | Best performing | 47.1 | 99.5 | 0.473 |
| SEQ ID NO: 98 | well | | | |
| C. maltosa | Two well | 3.3 ± 0.5 | 150.4 ± 8.9 | 0.022 |
| SEQ ID NO: 98 | average | | | |

### Example 5: Genetically Modified Strains-gapN and ADH

### Strains 2-1 Through 2-16

Strain 1-2 was transformed with the “5′ gapN and ADH Expression Cassette” and “3′ Selectable Marker Cassette” as indicated in Table 5 to produce strains 2-1 through 2-8. Each “5′ gapN and ADH Expression Cassette” contained i) a 5′ GPP1 flanking sequence; ii) a PGK1 promoter; iii) a gene encoding the indicated gapN enzyme; iv) a ADHI terminator; v) a TDH3 promoter; vi) a gene encoding the indicated ADH enzyme; vii) a CYC terminator; and viii) a 5′ portion of the ScURA open reading frame. The “3′ Selectable Marker Cassette” contained i) a 3′ GPP1 flanking sequence; ii) a loxP site; iii) a ScURA terminator; and vi) a 3′ portion of the ScURA open reading frame. Transformants were selected on ScD-Ura plates. Resulting transformants were streaked for single colony isolation on ScD-Ura plates. Single colonies were selected. Correct integration of the indicated gapN and ADH expression cassettes was verified by PCR and the PCR verified isolates were designated with the strain identifier outlined in Table 2. More than one PCR verified isolate, e.g., “sister” isolates, are indicated by letters following the strain number. For example, strain 2-1 has three sister isolates, strains 2-la, 2-1b, and 2-1c.

Resulting strains 2-1 through 2-8 included a single copy of the gapN and ADH expression cassettes in tandem on a single allele of the GPP1 gene.

### Strains 2-9 Through 2-16

Parent strains indicated in Table 5 were transformed with the “5′ gapN and ADH Expression Cassette” and “3′ Selectable Marker Cassette” as indicated in Table 5 to produce strains 2-9 through 2-16. Each “5′ gapN and ADH Expression Cassette” contained i) a 5′ GPP1 flanking sequence; ii) a PGK1 promoter; iii) a gene encoding the indicated gapN enzyme; iv) a ADHI terminator; v) a TDH3 promoter; vi) a gene encoding the indicated ADH enzyme; vii) a CYC terminator; and viii) a 5′ portion of the amdS open reading frame. The “3′ Selectable Marker Cassette” contained i) a 3′ GPP1 flanking sequence; ii) a loxP site; iii) an amdS terminator; and vi) a 3′ portion of the amdS open reading frame. Transformants were selected on YNB+acetamide plates. Resulting transformants were streaked for single colony isolation on YNB+acetamide plates. Single colonies were selected. Correct integration of the indicated gapN and ADH expression cassettes was verified by PCR and the PCR verified isolates were designated with the strain identifier outlined in Table 2. More than one PCR verified isolate, e.g., “sister” isolates, are indicated by letters following the strain number. For example, strain 2-9 has two sister isolates, strains 2-9a and 2-9b.

Resulting strains 2-9 through 2-16 included two copies of the gapN and ADH expression cassettes, one on each of the two alleles of the GPP1 gene.

TABLE 5

| | | 5′ gapN and ADH Expression Cassette | | | | 3′ Selectable | |
|---|---|---|---|---|---|---|---|
| | Parent | Transformation | | | | Marker Cassette | |
| Strain | Strain | SEQ ID NO: | gapN | ADH | Marker | SEQ ID NO: | Marker |
| 2-1a-c | 1-2 | 102 | S. pyogenes (SEQ ID NO: 28) | C. maltosa (SEQ ID NO: 98) | URA | 110 | URA |
| 2-2a-c | 1-2 | 103 | C. gleum (SEQ ID NO: 64) | R. toruloides (SEQ ID NO: 100) | URA | 110 | URA |
| 2-3a-c | 1-2 | 104 | C. gleum (SEQ ID NO: 64) | C. maltosa (SEQ ID NO: 98) | URA | 110 | URA |
| 2-4a-c | 1-2 | 105 | P. flourescens (SEQ ID NO: 32) | C. maltosa (SEQ ID NO: 98) | URA | 110 | URA |
| 2-5a-c | 1-2 | 106 | S. pyogenes (SEQ ID NO: 28) | R. toruloides (SEQ ID NO: 100) | URA | 110 | URA |
| 2-6a-c | 1-2 | 107 | A. thaliana (SEQ ID NO: 52) | R. toruloides (SEQ ID NO: 100) | URA | 110 | URA |
| 2-7a-c | 1-2 | 108 | A. thaliana (SEQ ID NO: 52) | C. maltosa (SEQ ID NO: 98) | URA | 110 | URA |
| 2-8a-c | 1-2 | 109 | P. flourescens (SEQ ID NO: 32) | R. toruloides (SEQ ID NO: 100) | URA | 110 | URA |
| 2-9a-b | 2-1a | 111 | S. pyogenes (SEQ ID NO: 28) | C. maltosa (SEQ ID NO: 98) | amdS | 119 | amdS |
| 2-10a-b | 2-2a | 112 | C. gleum (SEQ ID NO: 64) | R. toruloides (SEQ ID NO: 100) | amdS | 119 | amdS |
| 2-11a-c | 2-3c | 113 | C. gleum (SEQ ID NO: 64) | C. maltosa (SEQ ID NO: 98) | amdS | 119 | amdS |
| 2-12a-c | 2-5c | 114 | S. pyogenes (SEQ ID NO: 28) | R. toruloides (SEQ ID NO: 100) | amdS | 119 | amdS |
| 2-13a-c | 2-6c | 115 | A. thaliana (SEQ ID NO: 52) | R. toruloides (SEQ ID NO: 100) | amdS | 119 | amdS |
| 2-14a-b | 2-7c | 116 | A. thaliana (SEQ ID NO: 52) | C. maltosa (SEQ ID NO: 98) | amdS | 119 | amdS |
| 2-15a-c | 2-8b | 117 | P. flourescens (SEQ ID NO: 32) | R. toruloides (SEQ ID NO: 100) | amdS | 119 | amdS |
| 2-16a-c | 2-4a | 118 | P. flourescens (SEQ ID NO: 32) | C. maltosa (SEQ ID NO: 98) | amdS | 119 | amdS |

### Example 6: Shake Flask Assays

Strains 1-1, 1-3, and 2-1 through 2-8 were run in shake flasks to assay ethanol and glycerol production.

Strains were struck on a ScD-ura plate and incubated at 30° C. until single colonies were visible (2-3 days). Cells from the ScD-ura plate were scraped into sterile shake flask medium and the optical density (OD600) is measured. Optical density is measured at a wavelength of 600 nm with a 1 cm path length using a model Genesys 20 spectrophotometer (Thermo Scientific). A shake flask is inoculated with the cell slurry to reach an initial OD600 of 0.1. Immediately prior to inoculating, 50 mL of shake flask medium was added to a 250 mL baffled shake flask sealed with air-lock containing 4 ml of sterilized canola oil. The shake flask medium consisted of 725 g partially hydrolyzed corn starch, 150 g filtered sterilized (0.2 μm) light steep water, 10 g water, 25 g glucose, and 1 g urea. Strains were incubated at 30° C. with shaking in an orbital shake at 100 rpm for 72 hours. Samples were taken and analyzed for metabolite concentrations in the broth at the end of fermentation by HPLC.

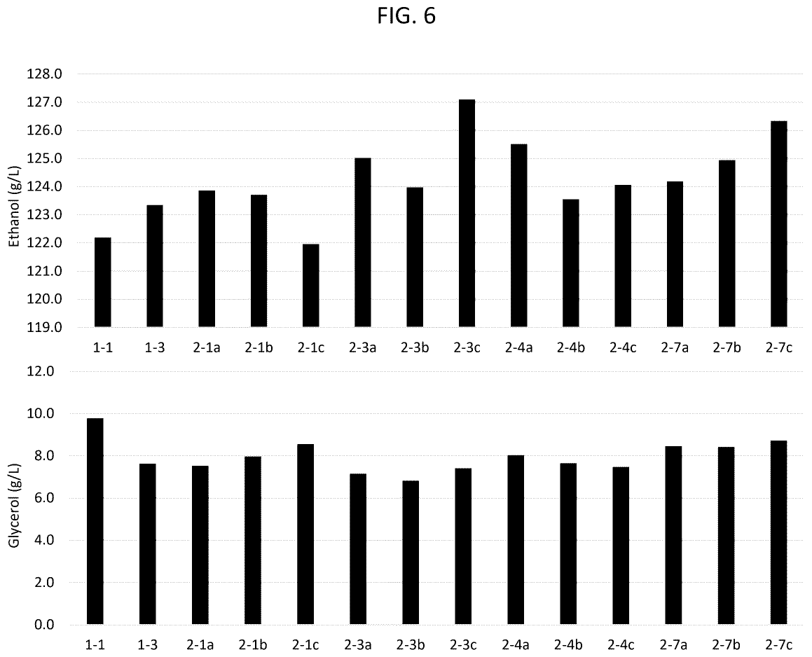

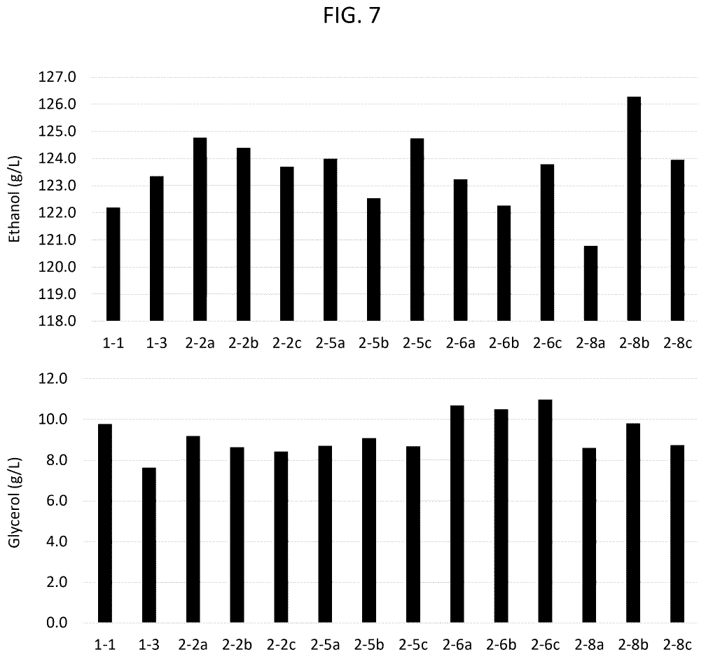

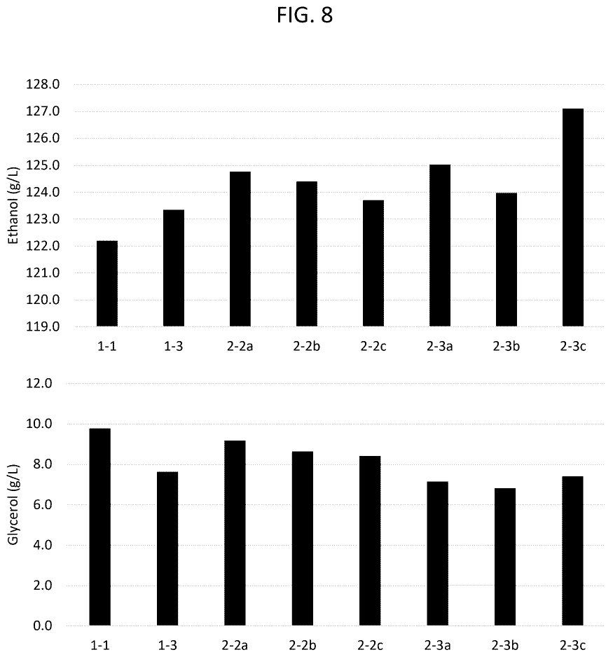

Fermentation results are reported in Tables 6-8 and in FIGS. 6-8. Many of the tested strains showed improved ethanol titers even through the glycerol titer was not reduced. This may be use to the increased utilization of the acetaldehyde with the NADPH-dependent ADH enzyme. Strains including the combination of the S. pyogenes gapN and the C. maltosa ADH showed the lowest residual glucose at the end of fermentation. Likewise, the strains including the combination of the C. gleum gapN with either the C. maltosa or R. toruloides ADH also showed lower glycerol and higher ethanol titers, despite higher residual glucose levels.

TABLE 6

| | Analyte (g/L) | | |
|---|---|---|---|
| Strain | Ethanol | Glycerol | Glucose |
| 1-1 | 122.188 | 9.772 | 1.006 |
| 1-3 | 123.340 | 7.622 | 0.949 |
| 2-1a | 123.859 | 7.523 | 0.995 |
| 2-1b | 123.704 | 7.956 | 0.987 |
| 2-1c | 121.952 | 8.544 | 0.962 |
| 2-3a | 125.014 | 7.144 | 0.941 |
| 2-3b | 123.968 | 6.816 | 0.903 |
| 2-3c | 127.095 | 7.402 | 0.988 |
| 2-4a | 125.505 | 8.021 | 1.095 |
| 2-4b | 123.547 | 7.643 | 0.940 |
| 2-4c | 124.056 | 7.466 | 0.905 |
| 2-7a | 124.181 | 8.447 | 0.949 |
| 2-7b | 124.934 | 8.413 | 0.959 |
| 2-7c | 126.326 | 8.717 | 1.049 |

TABLE 7

| | Analyte (g/L) | | |
|---|---|---|---|
| Strain | Ethanol | Glycerol | Glucose |
| 1-1 | 122.188 | 9.772 | 1.006 |
| 1-3 | 123.340 | 7.622 | 0.949 |
| 2-2a | 124.760 | 9.175 | 0.970 |
| 2-2b | 124.389 | 8.629 | 0.930 |
| 2-2c | 123.695 | 8.410 | 0.919 |
| 2-5a | 123.983 | 8.694 | 0.955 |
| 2-5b | 122.534 | 9.076 | 1.230 |
| 2-5c | 124.733 | 8.670 | 0.951 |
| 2-6a | 123.230 | 10.679 | 0.949 |
| 2-6b | 122.259 | 10.494 | 0.957 |
| 2-6c | 123.784 | 10.973 | 0.977 |
| 2-8a | 120.774 | 8.593 | 0.895 |
| 2-8b | 126.271 | 9.802 | 0.983 |
| 2-8c | 123.944 | 8.725 | 0.950 |

TABLE 8

| | Analyte (g/L) | | |
|---|---|---|---|
| Strain | Ethanol | Glycerol | Glucose |
| 1-1 | 122.188 | 9.772 | 1.006 |
| 1-3 | 123.340 | 7.622 | 0.949 |
| 2-2a | 124.760 | 9.175 | 0.970 |
| 2-2b | 124.389 | 8.629 | 0.930 |
| 2-2c | 123.695 | 8.410 | 0.919 |
| 2-3a | 125.014 | 7.144 | 0.941 |
| 2-3b | 123.968 | 6.816 | 0.903 |
| 2-3c | 127.095 | 7.402 | 0.988 |

### Example 7: Ambr15 Assays

Strains 1-1, 1-3, and 2-9 through 2-15 were run in an Ambr15 to assay ethanol and glycerol production.

Strains were struck on a ScD-ura or YPD plate and incubated at 30° C. until single colonies were visible (2-3 days). Cells from the ScD-ura or YPD plate were scraped into sterile medium and the optical density (OD600) is measured. Optical density is measured at a wavelength of 600 nm with a 1 cm path length using a model Genesys 20 spectrophotometer (Thermo Scientific). An Ambr15 reaction vessel is inoculated with the cell slurry to reach an initial OD600 of 0.2. The fermentation medium consisted of 295 g partially hydrolyzed corn starch, 90 g filtered sterilized (0.2 μm) light steep water, 79 g sterile water, and 36 g 500 g/L sterile glucose. With continuous stirring, 12 mL of fermentation media was added to each bioreactor. Strains were incubated at 30° C., 450 rpm of agitation. Air was supplied at 2.4 smlpm from the time of inoculation to 14 hours, then reduced to 0 smlpm for the remainder of the fermentation. Nitrogen gas was supplied at 0.01 smlpm from 14 hours until the end of fermentation. In experiments requiring exogenous glucoamylase, 45 μL of a 1:100 dilution of DuPont™ DISTILLASE® DXT was added at 14 hour after inoculation. Samples were taken and analyzed for ethanol and glycerol concentrations by HPLC.

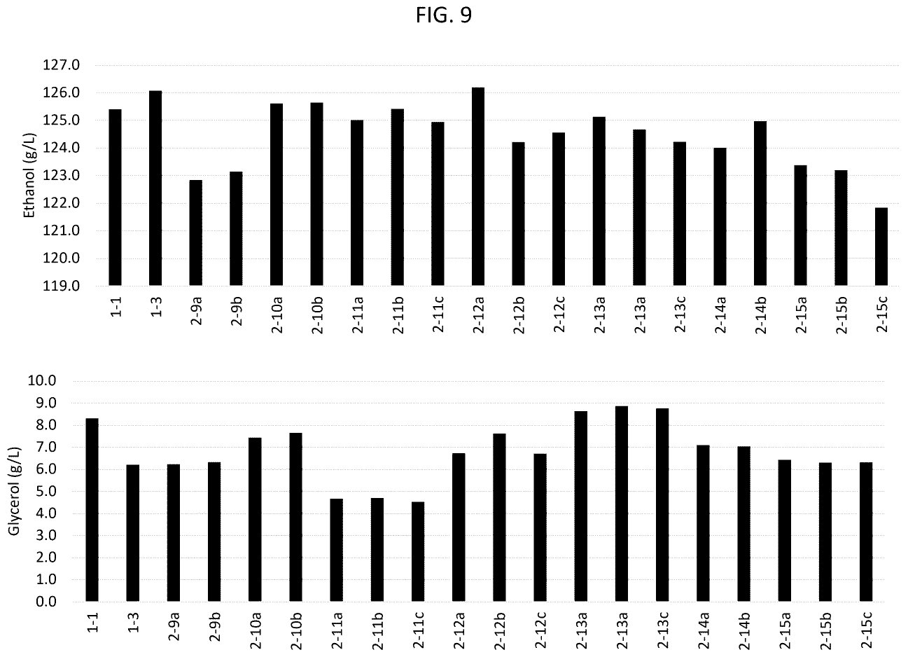

Results of the Ambr15 assays are reported in Table 9 and in FIG. 9. The only combination of enzymes that resulted in a lower glycerol titier compared to the positive control strain 1-3 was the combination of the C. gleum gapN and the C. maltosa ADH. The glycerol reduction of 25% between strain 1-3 and strains 2-11 is consistent with the results in Example 6. Glycerol reduction was improved by 45% relative to the parent strain 1-1. It is unclear why some strains resulted in higher glycerol levels than the parent strain 1-1. In general, ethanol titers were equivalent to or greater than both the parent (1-1) and positive control strains (1-3).

TABLE 9

| | |
|---|---|
| Analyte (g/L) | Ethanol Rate (g/(Lh)) |

| Strain | Ethanol | Glycerol | 0-14 h | 14 h-39 h |
|---|---|---|---|---|
| 1-1 | 125.399 | 8.301 | 2.070 | 3.436 |
| 1-3 | 126.074 | 6.202 | 2.124 | 2.300 |
| 2-9a | 122.832 | 6.224 | 1.460 | 3.637 |
| 2-9b | 123.139 | 6.319 | 1.760 | 3.568 |
| 2-10a | 125.606 | 7.430 | 0.851 | 3.994 |
| 2-10b | 125.636 | 7.639 | 1.310 | 3.669 |
| 2-11a | 125.006 | 4.664 | 1.118 | 3.475 |
| 2-11b | 125.412 | 4.694 | 0.776 | 3.676 |
| 2-11c | 124.934 | 4.524 | 0.565 | 3.693 |
| 2-12a | 126.189 | 6.723 | 0.602 | 4.345 |
| 2-12b | 124.208 | 7.614 | 1.989 | 3.498 |
| 2-12c | 124.555 | 6.701 | 2.044 | 3.495 |
| 2-13a | 125.126 | 8.630 | 1.327 | 3.841 |
| 2-13a | 124.667 | 8.857 | 1.831 | 3.501 |
| 2-13c | 124.220 | 8.754 | 1.620 | 3.824 |
| 2-14a | 124.005 | 7.094 | −0.117 | 4.567 |
| 2-14b | 124.961 | 7.034 | 1.652 | 3.592 |
| 2-15a | 123.372 | 6.426 | 1.693 | 3.714 |
| 2-15b | 123.195 | 6.297 | 1.636 | 3.714 |
| 2-15c | 121.832 | 6.307 | 1.743 | 3.658 |

1. A genetically engineered yeast cell capable of producing ethanol, the engineered yeast cell comprising
an exogenous polynucleotide encoding a glyceraldehyde-3-phosphate dehydrogenase (gapN) enzyme at least 80% identical to at least one of SEQ ID NOs: 64, 28, 32, 48, 52, 68, 80, 92, and 96.

2. The engineered yeast cell of claim 1, wherein the gapN enzyme is at least 85% identical to at least one of SEQ ID NOs: 64, 28, 32, 48, and 52.

3. The engineered yeast cell of claim 1, wherein the gapN enzyme is at least 90% identical to at least one of SEQ ID NOs: 64, 28, 32, and 52.

4. The engineered yeast cell of claim 1, wherein
the gapN enzyme is at least 85% identical to SEQ ID NO:64.

5. The engineered yeast cell of claim 1, wherein the engineered yeast cell additionally comprises an exogenous polynucleotide sequence encoding an alcohol dehydrogenase (ADH) enzyme at least 80% identical to at least one of SEQ ID NOs: 98 and 100.

6. The engineered yeast cell of claim 1, wherein the engineered yeast cell comprises a deletion or disruption of a native glycerol-3-phosphate phosphatase (GPP) gene.

7. The engineered yeast cell of claim 1, wherein the engineered yeast cell comprises a deletion or disruption of a native glycerol-3-phosphate dehydrogenase (GDP) gene.

8. The engineered yeast cell of claim 1, wherein the engineered yeast cell additionally comprises an exogenous polynucleotide sequence encoding a glucoamylase (GA) enzyme.

9. A genetically engineered yeast cell capable of producing ethanol, the engineered yeast cell comprising
an exogenous polynucleotide encoding a glyceraldehyde-3-phosphate dehydrogenase (gapN) enzyme at least 80% identical to at least one of SEQ ID NOs: 64, 28, 32, 48, 52, 68, 80, 92, and 96;
an exogenous polynucleotide sequence encoding an alcohol dehydrogenase (ADH) enzyme at least 80% identical to at least one of SEQ ID NOs: 98 and 100;
an exogenous polynucleotide sequence encoding a glucoamylase (GA) enzyme; and
a deletion or disruption of at least 1 allele of a native GPP gene.

10. The engineered yeast cell of claim 9, wherein the encoded GA enzyme is at least 80% identical to at least one of SEQ ID NOs: 122, 1, 123, and 124.

11. The engineered yeast cell of claim 1, wherein one or more of the exogenous polynucleotide sequences is operably linked to a heterologous or artificial promoter.

12. The engineered yeast cell of claim 11, wherein the promoter is selected from the group consisting of a pyruvate decarboxylase (PDC) promoter, a glyceraldehyde-3-phosphate dehydrogenase GAPDH (TDH3) promoter, a translation elongation factor 1 (TEF1) promoter, a URA3 promoter, an S-adenosyl methionine transferase 2 (SAM2) promoter; an alcohol dehydrogenase 1 (ADH1) promoter, and a 3-phosphoglycerate kinase (PGK1) promoter.

13. The engineered yeast cell of claim 1, wherein one or more of the exogenous polynucleotide sequences is operably linked to a heterologous or artificial terminator.

14. The engineered yeast cell of claim 13, wherein the terminator is selected from the group consisting of an iso-1-cytophrome c (CYC1) terminator, a URA3 terminator, a PDC terminator, an ADHI terminator, a TEF1 terminator, or a GAL10 terminator.

15. The engineered yeast cell of claim 1, wherein the yeast cell is selected from the group consisting of Saccharomyces spp., Schizosaccharomyces spp., Pichia spp., Paffia spp., Kluyveromyces spp., Candida spp., Talaromyces spp., Brettanomyces spp., Pachysolen spp., Debaryomyces spp., and Yarrowia spp.

16. The engineered yeast cell of claim 1, wherein the yeast cell is a Saccharomyces cerevisiae cell.

17. The engineered yeast cell of claim 1, wherein the engineered yeast cell is capable of producing ethanol at a titer of at least 60 g/L ethanol after 48 hours and wherein glycerol production by the engineered yeast cell is reduced relative to glycerol production in an equivalent yeast cell lacking the gapN enzyme.

18. A method for producing ethanol, the method comprising:
contacting a substrate with the engineered yeast cell of claim 1, where the engineered yeast cell produces at least 60 g/L ethanol after 48 hours and wherein glycerol production by the engineered yeast cell is reduced relative to glycerol production in an equivalent yeast cell lacking the gapN enzyme.

19. The method of claim 18, wherein the substrate comprises starch, glucose, sucrose, cellulosic biomass, or combinations thereof.

20. The method of claim 18, wherein the substrate is obtained from wheat, corn, or a combination thereof.
